(19) United States
(12) Patent Application Publication
(10) Pub. No.: US 2023/0010722 A1
(43) Pub. Date: Jan. 12, 2023

(54) BIOLOGICALLY-DERIVED FATTY ACIDS AND POLYMERS

(71) Applicant: The Regents of the University of California, Oakland, CA (US)

(72) Inventors: Michael D. Burkart, La Jolla, CA (US); Thien An Phung Hai, La Jolla, CA (US)

(21) Appl. No.: 17/765,347

(22) PCT Filed: Oct. 16, 2020
(86) PCT No.: PCT/US2020/056168

§ 371 Date: Mar. 30, 2022

Related U.S. Application Data
(60) Provisional application No. 62/916,413, filed on Oct. 17, 2019.

(51) Int. Cl. C08G 18/42; C11C 1/08; C11C 1/00; C11C 1/02; C07C 51/34; C07C 67/08; C08G 63/78; C08G 63/16; C08J 9/12; C11D 9/00

(52) U.S. Cl. CPC C08G 18/4288; C11C 1/08; C11C 1/007; C11C 1/025; C07C 51/34; C07C 67/08; C08G 63/78; C08G 63/16; C08J 9/125; C11D 9/007; C08G 2110/0008; C08G 2110/0083

(57) ABSTRACT

Disclosed herein, inter alia, are fatty acid and polymer compositions and methods of making the same.

### CROSS-REFERENCES TO RELATED APPLICATIONS

This application claims the benefit of U.S. Provisional Application No. 62/916,413, filed Oct. 17, 2019, which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with government support under grant no. DE-EE0008246 awarded by the Department of Energy. The government has certain rights in the invention.

### BACKGROUND

Monomer feedstocks and polymers made from such monomers are used in the synthesis of many products, including industrial polymers and plasticizers, foams, consumer products, hair and skin conditioners, fragrances and artificial flavors, drugs, and nutritional supplements. As resources such a petrochemicals become scarcer and with more emphasis on planet-friendly technologies, renewable polymers have become an important focus in next-generation materials. Particularly as the population grows, there is a need for more environmentally-friendly materials and methods of synthesis. Disclosed herein, inter alia, are solutions to these and other problems in the art.

### BRIEF SUMMARY

In an aspect is provided a method of producing an algae free fatty acid composition including:
(a) contacting an algae fatty acid hydrolysate with a base thereby forming a soap composition;
(b) washing the soap composition with an organic solvent thereby removing one or more algae pigments to form a washed soap composition;
(c) contacting the washed soap composition with an acid thereby forming an algae free fatty acid composition.

In an aspect is provided a composition including an aqueous phase and an organic phase, wherein the aqueous phase includes algae fatty acid alkali salts and the organic phase includes one or more algae pigments.

In an aspect is provided a composition including a palmitoleic acid (C16-1) alkali salt and a palmitic acid (C16-0) alkali salt.

In an aspect is provided a composition including a crystal phase and a liquid phase, wherein the crystal phase includes a urea crystal and palmitic acid (C16-0), and the liquid phase includes palmitoleic acid (C16-1).

In an aspect is provided a composition including palmitoleic acid (C16-1) and azelaic acid.

In an aspect is provided a composition including an algae polyester polymer and one or more of malonic acid, a 2-methylfumaric acid ester, and/or a terminal heptanoic acid ester.

In an aspect is provided a composition including an algae polyurethane and one or more of malonic acid, a 2-methylfumaric acid ester, or a terminal heptanoic acid ester.

### BRIEF DESCRIPTION OF THE DRAWINGS

FIG. 1. Pathway to foam polyurethane synthesis from algae oil.

FIG. 2. Fatty acids purification process.

FIG. 3. Urea complexation procedure.

FIG. 4. GC-MS of fatty acid methyl ester (FAME) from raw oil.

FIG. 5. Images of the raw oil (a) and purified oil (b).

FIG. 6. ¹H NMR spectrum of raw oil.

FIG. 7. ¹H NMR spectrum of purified oil.

FIG. 8. Fluorescence comparison of raw oil and purified oil.

FIGS. 9A-9B. GC-MS of (FIG. 9A) palmitoleic acid C16-1 and (FIG. 9B) palmitic acid C16-0.

FIGS. 10A-10B. ¹H NMR of (FIG. 10A) palmitoleic acid C16-1 and (FIG. 10B) palmitic acid C16-0.

FIG. 11. GC-MS of dimethyl azelate originate from synthesized azelaic acid.

FIGS. 12A-12B. ¹H NMR of synthesized azelaic acid (FIG. 12A) and heptanoic acid (FIG. 12B).

FIGS. 13A-13B. ¹³C NMR of synthesized azelaic acid (FIG. 13A) and heptanoic acid (FIG. 13B).

FIG. 14. ¹H NMR of polyester polyol.

FIG. 15. FT-IR of polyester polyol.

FIG. 16. DSC of polyester polyol.

FIG. 17. DSC of PU foam.

FIG. 18. Pathway to polyester polyol synthesis from algae oil.

FIG. 19. Fatty acids purification process.

FIG. 20. Schematic of the pigment removing system. 1—24/40 Friedrich's condenser; 2—standard thermometer; 3—34/45 to 24/40 glass reducing joint; 4—2 L pyrex bottle fitted male 24/40 joint and GL-14 thread adapter for thermometer; 5—105° 24/40 glass connecting elbow; 6—24/40 GL-14 thermometer adapter; 7—1 L 24/40 20° two-neck roundbottom flask; 8—controlled heat/stir plate or mantle.

FIG. 21. Operational diagram for pigments removing.

FIG. 22. Schematic view of the ozonolysis of C16-1.

FIG. 23. Pathway to renewable polyurethane, n-hexane, and methyl heptanoate from algae biomass.

FIG. 24. Synthesis of methyl heptanoate and hexane from heptanoic acid.

FIGS. 25A-25B. Images of palmitoleic acid C16-1 (FIG. 25A) and palmitic acid C16-0 (FIG. 25B).

FIG. 26. GC-MS chromatogram of obtained hexane and internal standard.

FIG. 27. Equation used to calculate δ¹³C.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The symbol “ [symbol] ” denotes the point of attachment of a chemical moiety to the remainder of a molecule or chemical formula.

As used herein, the term “isomers” refers to compounds having the same number and kind of atoms, and hence the same molecular weight, but differing in respect to the structural arrangement or configuration of the atoms.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

The terms “a” or “an,” as used in herein means one or more.

A “detectable agent” or “detectable moiety” is an atom, molecule, substance, or composition detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, propionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g., signaling pathway) of a protein in the absence of a compound as described herein (including embodiments, examples, figures, or Tables).

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a cellular component (e.g., protein, ion, lipid, nucleic acid, nucleotide, amino acid, protein, particle, organelle, cellular compartment, microorganism, virus, lipid droplet, vesicle, small molecule, protein complex, protein aggregate, or macromolecule). In some embodiments contacting includes allowing a compound described herein to interact with a cellular component (e.g., protein, ion, lipid, nucleic acid, nucleotide, amino acid, protein, particle, virus, lipid droplet, organelle, cellular compartment, microorganism, vesicle, small molecule, protein complex, protein aggregate, or macromolecule) that is involved in a signaling pathway.

The term “biologically-derived” as used herein in reference to a compound, composition or molecule refers to a compound, composition or molecule that is obtained from an organism. In some cases, the organism is a photosynthetic organism.

The term “fatty acid” is used in accordance with its plain ordinary meaning and refers to a carboxylic acid with a long alkyl chain, which is either saturated or unsaturated. In embodiments, the long alkyl chain is a C₄-C₃₀ alkyl chain. In embodiments, the long alkyl chain is a C₄-C₂₈ alkyl chain. In embodiments, the long alkyl chain is a C₄-C₂₀ alkyl chain. In embodiments, the long alkyl chain is a C₁₀-C₃₀ alkyl chain. In embodiments, the long alkyl chain is a C₁₀-C₂₀ alkyl chain. In embodiments, the long alkyl chain is a C₁₆-C₃₀ alkyl chain. In embodiments, the long alkyl chain is a C₁₆-C₂₅ alkyl chain. In embodiments, the long alkyl chain is a C₁₆-C₂₀ alkyl chain. In embodiments, the fatty acid is in an ester form. In embodiments, the fatty acid is in a triglyceride form. In embodiments, the fatty acid is in a phospholipid form. In embodiments, the fatty acid is in a cholesteryl ester form.

The term “free fatty acid” is used in accordance with its plain ordinary meaning and refers to a non-esterified fatty acid. In embodiments, the free fatty acid is palmitic acid (C16-0). In embodiments, the free fatty acid is palmitoleic acid (C16-1). A “free fatty acid composition” as used herein refers to a composition including a free fatty acid.

The term “algae free fatty acid composition” as used herein refers to a free fatty acid composition derived from algae. In embodiments, the algae is Nannochloropsis sp.

The term “crystalline fatty acid composition” as used herein refers to a crystalline compound, wherein the crystalline compound includes a fatty acid compound. In embodiments, the fatty acid compound is partially or fully encompassed within the crystalline compound. In embodiments, the crystalline compound includes urea. In embodiments, the crystalline compound includes thiourea.

The term “soap composition” as used herein refers to the resulting composition following the reaction of a fatty acid hydrolysate and a base. A “fatty acid hydrolysate” refers to a fatty acid that has undergone a hydrolysis reaction. An “algae fatty acid hydrolysate” refers to a fatty acid hydrolysate derived from algae. In embodiments, the algae is Nannochloropsis sp.

The term “washed soap composition” as used herein refers to the resulting composition following washing a soap composition with an organic solvent.

The term “algae pigments” as used herein refers to colored organic compounds and organic cofactors found in algae. In embodiments, the algae pigment is a colored organic compound. In embodiments, the algae pigment is a chlorophyll. In embodiments, the algae pigment is a carotenoid. In embodiments, the algae pigment is a phycobilin. In embodiments, the organic cofactor is ubiquinone (i.e., coenzyme Q). in embodiments, the organic cofactor is tocochromanols (e.g., vitamin E). In embodiments, the algae is Nannochloropsis sp.

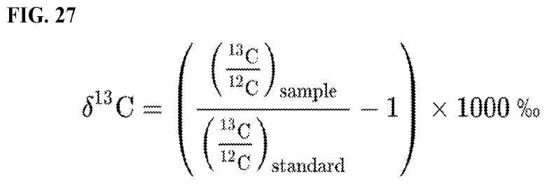

The term “δ¹³C” is used in accordance with its plain ordinary meaning in the art and refers to a measure of the ratio of ¹³C to ¹²C, reported in parts per thousand (per mil, ‰). The equation used to calculate δ¹³C is shown in FIG. 27.

### II. Methods of Making

In an aspect is provided a method of producing an algae free fatty acid composition. The method includes contacting an algae fatty acid hydrolysate with a base thereby forming a soap composition. The soap composition is washed, thereby removing one or more algae pigments to form a washed soap composition. The washed soap composition is contacted with an acid, thereby forming an algae free fatty acid composition.

In embodiments, the algae fatty acid hydrolysate is derived from a whole algae cell. In embodiments, the whole algae cell includes algae pigments.

In embodiments, the algae free fatty acid composition includes C16 free fatty acids. In embodiments, the C16 free fatty acids include palmitic (C16-0) free fatty acids and palmitoleic (C16-1) free fatty acids.

In embodiments, the base is sodium hydroxide, potassium hydroxide, lithium hydroxide, or calcium hydroxide. In embodiments, the base is sodium hydroxide. In embodiments, the base is potassium hydroxide. In embodiments, the base is lithium hydroxide. In embodiments, the base is calcium hydroxide.

In embodiments, the organic solvent is acetone, ether, or methyl tert-butyl ether. In embodiments, the organic solvent is acetone. In embodiments, the organic solvent is ether. In embodiments, the organic solvent is methyl tert-butyl ether.

In embodiments, the one or more algae pigments are selected from the group consisting of a chlorophyll, a carotenoid, and a chlorophyll degradation product. In embodiments, the one or more algae pigments is selected from the group consisting of chlorophyll, astaxanthin, zeaxanthin, and canthaxanthin. In embodiments, the algae pigment is a chlorophyll. In embodiments, the algae pigment is a carotenoid. In embodiments, the algae pigment is a chlorophyll degradation product. In embodiments, the algae pigment is an astaxanthin. In embodiments, the algae pigment is a zeaxanthin. In embodiments, the algae pigment is a canthaxanthin.

In embodiments, the method further includes removing one or more saturated fatty acids from the algae fatty acid composition thereby forming an algae unsaturated fatty acid composition. In embodiments, the removing includes contacting the algae fatty acid composition with methanol and applying temperature of 0° C. to −20° C., thereby forming a crystalline fatty acid composition. In embodiments, the removing includes contacting the algae fatty acid composition with methanol and applying temperature of 0° C. to −15° C., thereby forming a crystalline fatty acid composition. In embodiments, the removing includes contacting the algae fatty acid composition with methanol and applying temperature of −5° C. to −20° C., thereby forming a crystalline fatty acid composition. In embodiments, the removing includes contacting the algae fatty acid composition with methanol and applying temperature of −5° C. to −15° C., thereby forming a crystalline fatty acid composition. In embodiments, the removing includes contacting the algae fatty acid composition with methanol and applying temperature of −10° C. to −20° C., thereby forming a crystalline fatty acid composition. In embodiments, the removing includes contacting the algae fatty acid composition with methanol and applying temperature of −10° C. to −15° C., thereby forming a crystalline fatty acid composition. In embodiments, the removing includes contacting the algae fatty acid composition with methanol and applying temperature of −15° C., thereby forming a crystalline fatty acid composition. In embodiments, the removing includes contacting the algae fatty acid composition with methanol and applying temperature of about 0° C. to about −20° C., thereby forming a crystalline fatty acid composition. In embodiments, the removing includes contacting the algae fatty acid composition with methanol and applying temperature of about 0° C. to about −15° C., thereby forming a crystalline fatty acid composition. In embodiments, the removing includes contacting the algae fatty acid composition with methanol and applying temperature of about −5° C. to about −20° C., thereby forming a crystalline fatty acid composition. In embodiments, the removing includes contacting the algae fatty acid composition with methanol and applying temperature of about −5° C. to about −15° C., thereby forming a crystalline fatty acid composition. In embodiments, the removing includes contacting the algae fatty acid composition with methanol and applying temperature of about −10° C. to about −20° C., thereby forming a crystalline fatty acid composition. In embodiments, the removing includes contacting the algae fatty acid composition with methanol and applying temperature of about −10° C. to about −15° C., thereby forming a crystalline fatty acid composition. In embodiments, the removing includes contacting the algae fatty acid composition with methanol and applying temperature of about −15° C., thereby forming a crystalline fatty acid composition. In embodiments, the crystalline fatty acid composition includes a saturated fatty acid. In embodiments, the removing includes contacting the algae fatty acid composition with urea at a temperature of 0° C. to 5° C., thereby forming a crystalline fatty acid composition. In embodiments, the removing includes contacting the algae fatty acid composition with urea at a temperature of 0° C., thereby forming a crystalline fatty acid composition. In embodiments, the removing includes contacting the algae fatty acid composition with urea at a temperature of 1° C., thereby forming a crystalline fatty acid composition. In embodiments, the removing includes contacting the algae fatty acid composition with urea at a temperature of 2° C., thereby forming a crystalline fatty acid composition. In embodiments, the removing includes contacting the algae fatty acid composition with urea at a temperature of 3° C., thereby forming a crystalline fatty acid composition. In embodiments, the removing includes contacting the algae fatty acid composition with urea at a temperature of 4° C., thereby forming a crystalline fatty acid composition. In embodiments, the removing includes contacting the algae fatty acid composition with urea at a temperature of 5° C., thereby forming a crystalline fatty acid composition. In embodiments, the removing includes contacting the algae fatty acid composition with urea at a temperature of about 0° C. to about 5° C., thereby forming a crystalline fatty acid composition. In embodiments, the removing includes contacting the algae fatty acid composition with urea at a temperature of about 0° C., thereby forming a crystalline fatty acid composition. In embodiments, the removing includes contacting the algae fatty acid composition with urea at a temperature of about 1° C., thereby forming a crystalline fatty acid composition. In embodiments, the removing includes contacting the algae fatty acid composition with urea at a temperature of about 2° C., thereby forming a crystalline fatty acid composition. In embodiments, the removing includes contacting the algae fatty acid composition with urea at a temperature of about 3° C., thereby forming a crystalline fatty acid composition. In embodiments, the removing includes contacting the algae fatty acid composition with urea at a temperature of about 4° C., thereby forming a crystalline fatty acid composition. In embodiments, the removing includes contacting the algae fatty acid composition with urea at a temperature of about 5° C., thereby forming a crystalline fatty acid composition. In embodiments, the crystalline fatty acid composition includes crystalline urea and a saturated fatty acid. In embodiments, the crystalline fatty acid composition includes a complex including a saturated fatty acid enclosed within crystalline urea. In embodiments, the saturated fatty acid is palmitic acid (C16-0).

In embodiments, the algae fatty acid hydrolysate is a microalgae fatty acid hydrolysate. In embodiments, the algae fatty acid hydrolysate is a Nannochloropsis sp. fatty acid hydrolysate, a Chlamydomonas sp. fatty acid hydrolysate, a Dunaliella sp. fatty acid hydrolysate, a Haematococcus sp. fatty acid hydrolysate, a Scenedesmus sp. fatty acid hydrolysate, a Diaphoreolis sp. fatty acid hydrolysate, or a Dunaliella sp. fatty acid hydrolysate. In embodiments, the algae fatty acid hydrolysate is a Nannochloropsis sp. fatty acid hydrolysate. In embodiments, the algae fatty acid hydrolysate is a Chlamydomonas sp. fatty acid hydrolysate. In embodiments, the algae fatty acid hydrolysate is a Dunaliella sp. fatty acid hydrolysate. In embodiments, the algae fatty acid hydrolysate is a Haematococcus sp. fatty acid hydrolysate. In embodiments, the algae fatty acid hydrolysate is a Scenedesmus sp. fatty acid hydrolysate. In embodiments, the algae fatty acid hydrolysate is a Diaphoreolis sp. fatty acid hydrolysate. In embodiments, the algae fatty acid hydrolysate is a Dunaliella sp. fatty acid hydrolysate.

In embodiments, the algae fatty acid hydrolysate is a C. reinhardtii fatty acid hydrolysate, D. satina fatty acid hydrolysate, H. pluvatis fatty acid hydrolysate, S. dimorphus fatty acid hydrolysate, D. viridis fatty acid hydrolysate, D. tertiolecta fatty acid hydrolysate, N. oculata fatty acid hydrolysate, or N. salina fatty acid hydrolysate. In embodiments, the algae fatty acid hydrolysate is a C. reinhardtii fatty acid hydrolysate. In embodiments, the algae fatty acid hydrolysate is a D. satina fatty acid hydrolysate. In embodiments, the algae fatty acid hydrolysate is a H. pluvatis fatty acid hydrolysate. In embodiments, the algae fatty acid hydrolysate is a S. dimorphus fatty acid hydrolysate. In embodiments, the algae fatty acid hydrolysate is a D. viridis fatty acid hydrolysate. In embodiments, the algae fatty acid hydrolysate is a D. tertiolecta fatty acid hydrolysate. In embodiments, the algae fatty acid hydrolysate is a N. oculata fatty acid hydrolysate. In embodiments, the algae fatty acid hydrolysate is a N. salina fatty acid hydrolysate.

In embodiments, the algae fatty acid hydrolysate is a Cyanophyta fatty acid hydrolysate, a Prochlorophyta fatty acid hydrolysate, a Rhodophyta fatty acid hydrolysate, a Chlorophyta fatty acid hydrolysate, a Heterokontophyta fatty acid hydrolysate, a Tribophyta fatty acid hydrolysate, a Glaucophyta fatty acid hydrolysate, a Chlorarachniophyte fatty acid hydrolysate, a Euglenophyta fatty acid hydrolysate, a Euglenoid fatty acid hydrolysate, a Haptophyta fatty acid hydrolysate, a Chrysophyta fatty acid hydrolysate, a Cryptophyta fatty acid hydrolysate, a Cryptomonad fatty acid hydrolysate, a Dinophyta fatty acid hydrolysate, a Dinoflagellata fatty acid hydrolysate, a Prymnesiophyta fatty acid hydrolysate, a Bacillariophyta fatty acid hydrolysate, a Xanthophyta fatty acid hydrolysate, a Eustigmatophyta fatty acid hydrolysate, a Raphidophyta fatty acid hydrolysate, a Phaeophyta fatty acid hydrolysate, or a Phytoplankton fatty acid hydrolysate.

In embodiments, the algae unsaturated fatty acid composition includes palmitoleic acid (C16-1).

In embodiments, the purity of the palmitoleic acid (C16-1) is a percentage of the total amount (e.g., mass) of the algae unsaturated fatty acid composition. In embodiments, the purity of the palmitoleic acid (C16-1) is a percentage of the total amount (e.g., mass) of unsaturated fatty acids in the algae unsaturated fatty acid composition. In embodiments, the purity of the palmitoleic acid (C16-1) is from 80% to 99%. In embodiments, the purity of the palmitoleic acid (C16-1) is from 91% to 99%. In embodiments, the purity of the palmitoleic acid (C16-1) is from 80% to 95%. In embodiments, the purity of the palmitoleic acid (C16-1) is from 85% to 95%. In embodiments, the purity of the palmitoleic acid (C16-1) is from 85% to 90%. In embodiments, the purity of the palmitoleic acid (C16-1) is 85%. In embodiments, the purity of the palmitoleic acid (C16-1) is 86%. In embodiments, the purity of the palmitoleic acid (C16-1) is 87%. In embodiments, the purity of the palmitoleic acid (C16-1) is 88%. In embodiments, the purity of the palmitoleic acid (C16-1) is 89%. In embodiments, the purity of the palmitoleic acid (C16-1) is 90%. In embodiments, the purity of the palmitoleic acid (C16-1) is 91%. In embodiments, the purity of the palmitoleic acid (C16-1) is 92%. In embodiments, the purity of the palmitoleic acid (C16-1) is 93%. In embodiments, the purity of the palmitoleic acid (C16-1) is 94%. In embodiments, the purity of the palmitoleic acid (C16-1) is 95%. In embodiments, the purity of the palmitoleic acid (C16-1) is 96%. In embodiments, the purity of the palmitoleic acid (C16-1) is 97%. In embodiments, the purity of the palmitoleic acid (C16-1) is 98%. In embodiments, the purity of the palmitoleic acid (C16-1) is 99%. In embodiments, the purity of the palmitoleic acid (C16-1) is 100%. In embodiments, the purity of the palmitoleic acid (C16-1) is 90%. In embodiments, the purity of the palmitoleic acid (C16-1) is from about 80% to about 99%. In embodiments, the purity of the palmitoleic acid (C16-1) is from about 91% to about 99%. In embodiments, the purity of the palmitoleic acid (C16-1) is from about 80% to about 95%. In embodiments, the purity of the palmitoleic acid (C16-1) is from about 85% to about 95%. In embodiments, the purity of the palmitoleic acid (C16-1) is from about 85% to about 90%. In embodiments, the purity of the palmitoleic acid (C16-1) is about 85%. In embodiments, the purity of the palmitoleic acid (C16-1) is about 86%. In embodiments, the purity of the palmitoleic acid (C16-1) is about 87%. In embodiments, the purity of the palmitoleic acid (C16-1) is about 88%. In embodiments, the purity of the palmitoleic acid (C16-1) is about 89%. In embodiments, the purity of the palmitoleic acid (C16-1) is about 90%. In embodiments, the purity of the palmitoleic acid (C16-1) is about 91%. In embodiments, the purity of the palmitoleic acid (C16-1) is about 92%. In embodiments, the purity of the palmitoleic acid (C16-1) is about 93%. In embodiments, the purity of the palmitoleic acid (C16-1) is about 94%. In embodiments, the purity of the palmitoleic acid (C16-1) is about 95%. In embodiments, the purity of the palmitoleic acid (C16-1) is about 96%. In embodiments, the purity of the palmitoleic acid (C16-1) is about 97%. In embodiments, the purity of the palmitoleic acid (C16-1) is about 98%. In embodiments, the purity of the palmitoleic acid (C16-1) is about 99%. In embodiments, the purity of the palmitoleic acid (C16-1) is about 100%. In embodiments, the purity of the palmitoleic acid is determined by reacting unsaturated fatty acids in the algae unsaturated fatty acid composition to form corresponding unsaturated fatty acid methyl esters (e.g., via Fischer esterification), followed by analysis of the unsaturated fatty acid resulting methyl esters by gas chromography-mass spectrometry.

In embodiments, the method further includes contacting the algae unsaturated fatty acid composition with ozone followed by oxidation thereby forming an algae saturated dicarboxylic acid composition. In embodiments, the algae saturated dicarboxylic acid composition is an azelaic acid composition.

In embodiments, the algae unsaturated fatty acid composition is contacted with ozone in an aqueous-organic solvent system. In embodiments, the aqueous-organic solvent system includes aqueous acetonitrile.

In embodiments, the oxidation is performed by the addition of aqueous sodium chlorite, followed by a quenching step with an aqueous reductant. In embodiments, the aqueous reductant is aqueous sodium bisulfite.

In embodiments, the algae saturated dicarboxylic acid composition includes heptanoic acid. In embodiments, the method further includes decarboxylating the heptanoic acid. In embodiments, the decarboxylating includes catalytic hydrogenation or a light-dependent catalytic decarboxylation. In embodiments, the method further includes esterifying the heptanoic acid to heptanoyl methyl ester. In embodiments, the esterifying is performed under Fischer esterification conditions.

In embodiments, the method further includes contacting the algae saturated dicarboxylic acid composition with a diol thereby forming an algae polyester polymer composition. In embodiments, the algae polyester polymer composition is formed by a Fischer esterification of the dicarboxylic acid and the diol.

In embodiments, the algae polyester polymer composition is formed using an esterification catalyst selected from the group consisting of a metal chloride, a metal oxide, a metal carboxylate, a metal alkoxide and an organic acid. In embodiments, the esterification catalyst is a metal chloride. In embodiments, the esterification catalyst is a metal oxide. In embodiments, the esterification catalyst is a metal carboxylate. In embodiments, the esterification catalyst is a metal alkoxide. In embodiments, the esterification catalyst is an organic acid.

In embodiments, the esterification catalyst is zinc chloride. In embodiments, the esterification catalyst is aluminum chloride. In embodiments, the esterification catalyst is tin chloride. In embodiments, the esterification catalyst is copper chloride. In embodiments, the esterification catalyst is zinc oxide. In embodiments, the esterification catalyst is aluminum oxide. In embodiments, the esterification catalyst is tin oxide. In embodiments, the esterification catalyst is copper oxide. In embodiments, the esterification catalyst is a zinc carboxylate salt (e.g., zinc acetate, zinc laurate, or zinc octoate). In embodiments, the esterification catalyst is an aluminum carboxylate salt (e.g., aluminum acetate, aluminum laurate, or aluminum octoate). In embodiments, the esterification catalyst is a tin carboxylate salt (e.g., tin acetate, tin laurate, or tin octoate). In embodiments, the esterification catalyst is a copper carboxylate salt (e.g., copper acetate, copper laurate, or copper octoate). In embodiments, the esterification catalyst is a sodium alkoxide (e.g., sodium ethoxide). In embodiments, the esterification catalyst is a titanium alkoxide (e.g., tetrabutyltitanate). In embodiments, the esterification catalyst is a aluminum alkoxide (e.g., aluminum triisopropoxide). In embodiments, the esterification catalyst is p-toluenesulfonic acid.

In embodiments, the esterification catalyst is 0.0001 wt % to 0.1 wt % relative to the weight of the algae saturated dicarboxylic acid composition. In embodiments, the esterification catalyst is 0.001 wt % to 0.1 wt % relative to the weight of the algae saturated dicarboxylic acid composition. In embodiments, the esterification catalyst is 0.01 wt % to 0.1 wt % relative to the weight of the algae saturated dicarboxylic acid composition. In embodiments, the esterification catalyst is 0.0001 wt % to 0.05 wt % relative to the weight of the algae saturated dicarboxylic acid composition. In embodiments, the esterification catalyst is 0.001 wt % to 0.05 wt % relative to the weight of the algae saturated dicarboxylic acid composition. In embodiments, the esterification catalyst is 0.01 wt % to 0.05 wt % relative to the weight of the algae saturated dicarboxylic acid composition.

In embodiments, the algae saturated dicarboxylic acid composition is an algae azelaic acid composition and the diol is ethylene glycol. In embodiments, the algae saturated dicarboxylic acid composition is an algae oxalic acid composition, algae malonic acid composition, algae succinic acid composition, algae glutaric acid composition, algae adipic acid composition, algae 2,5-furandicarboxyic acid composition, algae pimelic acid composition, algae 3,3-dimethyl-1,2-cyclopropanedicarboxylic acid composition, algae suberic acid composition, algae azelaic acid composition, or algae sebacic acid composition. In embodiments, the algae saturated dicarboxylic acid composition is an algae azelaic acid composition. In embodiments, the algae saturated dicarboxylic acid composition is an algae oxalic acid composition. In embodiments, the algae saturated dicarboxylic acid composition is an algae malonic acid composition. In embodiments, the algae saturated dicarboxylic acid composition is an algae succinic acid composition. In embodiments, the algae saturated dicarboxylic acid composition is an algae glutaric acid composition. In embodiments, the algae saturated dicarboxylic acid composition is an algae adipic acid composition. In embodiments, the algae saturated dicarboxylic acid composition is an algae 2,5-furandicarboxyic acid composition. In embodiments, the algae saturated dicarboxylic acid composition is an algae pimelic acid composition. In embodiments, the algae saturated dicarboxylic acid composition is an algae 3,3-dimethyl-1,2-cyclopropanedicarboxylic acid composition. In embodiments, the algae saturated dicarboxylic acid composition is an algae suberic acid composition. In embodiments, the algae saturated dicarboxylic acid composition is an algae azelaic acid composition. In embodiments, the algae saturated dicarboxylic acid composition is an algae sebacic acid composition. In embodiments, the diol is selected from the group consisting of ethylene glycol, 1,2 propanediol, 1,3-propanediol, glycerol, 1,3-butanediol, 1,4-butanediol, 2-methyl-1,3-propanediol, 2,3-butanediol, trimethylolpropane, 1,5-pentanediol, 1,6-hexanediol, 3-methyl-1,5-pentanediol, 1,7-heptanediol, 1,8-octanediol, 1,9-nonanediol, and 1,10-decanediol. In embodiments, the diol is ethylene glycol. In embodiments, the diol is 1,2 propanediol. In embodiments, the diol is 1,3-propanediol. In embodiments, the diol is glycerol. In embodiments, the diol is 1,3-butanediol. In embodiments, the diol is 1,4-butanediol. In embodiments, the diol is 2-methyl-1,3-propanediol. In embodiments, the diol is 2,3-butanediol. In embodiments, the diol is trimethylolpropane. In embodiments, the diol is 1,5-pentanediol. In embodiments, the diol is 1,6-hexanediol. In embodiments, the diol is 3-methyl-1,5-pentanediol. In embodiments, the diol is 1,7-heptanediol. In embodiments, the diol is 1,8-octanediol. In embodiments, the diol is 1,9-nonanediol. In embodiments, the diol is 1,10-decanediol.

In embodiments, the method further includes contacting the algae polyester polymer composition with a diisocyanate to form an algae polyurethane composition. In embodiments, the diisocyanate is selected from the group consisting of toluene diisocyanate (TDI), methylene diphenyl diisocyanate (MDI), hexamethylene diisocyanate (HDI), pentamethylene diisocyanate (PDI), 2,5-furandiisocyanate (FDI), heptamethylene diisocyanate (HPDI), hydrogenated MDI (H12MDI), and isophorone diisocyanate (IPDI). In embodiments, the diisocyanate is toluene diisocyanate (TDI). In embodiments, the diisocyanate is methylene diphenyl diisocyanate (MDI). In embodiments, the diisocyanate is hexamethylene diisocyanate (HDI). In embodiments, the diisocyanate is pentamethylene diisocyanate (PDI). In embodiments, the diisocyanate is 2,5-furandiisocyanate (FDI). In embodiments, the diisocyanate is heptamethylene diisocyanate (HPDI). In embodiments, the diisocyanate is hydrogenated MDI (H12MDI). In embodiments, the diisocyanate is isophorone diisocyanate (IPDI).

In embodiments, the algae polyurethane composition is formed using a catalyst selected from the group consisting of triethylenediamine, bis-(2-dimethylaminoethyl)-ether, N-methylmorpholine, n-ethylmorpholine, N,N,N′-trimethylisopropyl propylenediamine, dimethylcyclohexylamine, 1-methyl-4-dimethylaminoethylpiperazine, methoxypropyldimethylamine, N,N,N′,N′-tetramethyl-1,3-butanediamine, and dimethylethanolamine. In embodiments, the catalyst used to form the algae polyurethane composition is triethylenediamine. In embodiments, the catalyst used to form the algae polyurethane composition is bis-(2-dimethylaminoethyl)-ether. In embodiments, the catalyst used to form the algae polyurethane composition is N-methylmorpholine. In embodiments, the catalyst used to form the algae polyurethane composition is N-ethylmorpholine. In embodiments, the catalyst used to form the algae polyurethane composition is N,N,N′-trimethylisopropyl propylenediamine. In embodiments, the catalyst used to form the algae polyurethane composition is dimethylcyclohexylamine. In embodiments, the catalyst used to form the algae polyurethane composition is 1-methyl-4-dimethylaminoethylpiperazine. In embodiments, the catalyst used to form the algae polyurethane composition is methoxypropyldimethylamine. In embodiments, the catalyst used to form the algae polyurethane composition is N,N,N′N′-tetramethyl-1,3-butanediamine. In embodiments, the catalyst used to form the algae polyurethane composition is dimethylethanolamine.

In embodiments, the method of forming the algae polyurethane composition further includes surfactants, cell regulators, cell openers, viscosity modifiers, and/or plasticizers. In embodiments, the surfactant is Vorasurf DC193. In embodiments, the surfactant is DABCO DC5179. In embodiments, the surfactant is Niax L-620. In embodiments, the surfactant is Niax L-1500. In embodiments, the surfactant is Niax L-3001. In embodiments, the surfactant is Niax L-5440. In embodiments, the surfactant is Tegostab BF 2270. In embodiments, the surfactant is Tegostab 8680. In embodiments, the cell regulator is Niax L-1501. In embodiments, the cell regulator is Niax L-1507. In embodiments, the cell regulator is Vorasurf DC3043. In embodiments, the cell regulator is DABCO DC193. In embodiments, the cell regulator is Tegostab B4351. In embodiments, the cell regulator is Tegostab B4690. In embodiments, the cell opener is Voranol CP1421. In embodiments, the cell opener is Voranol 4053. In embodiments, the cell opener is Gorapur IMR 852. In embodiments, the cell opener is Tegostab B8948. In embodiments, the cell opener is Niax L-6164. In embodiments, the cell opener is Niax L-6186. In embodiments, the cell opener is and Niax L-6189. In embodiments, the viscosity modifier is ethylene carbonate. In embodiments, the viscosity modifier is propylene carbonate. In embodiments, the viscosity modifier is a polyoxyalkylene derivative (e.g., triethylene glycol monobutyl ether). In embodiments, the plasticizer is a dibasic ester (e.g., dipropyleneglycol dibenzoate, dimethyl succinate, dimethyl glutarate, or dimethyl adipate, or mixtures thereof). In embodiments, the plasticizer is an epoxidized soybean oil derivative. In embodiments, the plasticizer is a linseed oil derivative.

In an aspect is provided a method of preparing or synthesizing a compound described herein. In embodiments, the compound described herein is synthesized from algae oil. In embodiments, the method includes removing pigments from algae oil. In embodiments, the method includes ozonolysis of palmitoleic acid. In embodiments, the ozonolysis is conducted using continuous flow technology.

### III. Compounds and Compositions

In an aspect is provided a composition including an aqueous phase and an organic phase, wherein the aqueous phase includes algae fatty acid alkali salts and the organic phase includes one or more algae pigments (e.g., as described herein).

In embodiments, the algae fatty acid alkali salt is an eicosapentaenoic acid (C20-5) alkali salt, a palmitoleic acid (C16-1) alkali salt, and/or a palmitic acid (C16-0) alkali salt. In embodiments, the algae fatty acid alkali salt is an eicosapentaenoic acid (C20-5) alkali salt. In embodiments, the algae fatty acid alkali salt is a palmitoleic acid (C16-1) alkali salt. In embodiments, the algae fatty acid alkali salt is a palmitic acid (C16-0) alkali salt.

In embodiments, the one or more algae pigments is selected from the group consisting of chlorophyll, astaxanthin, zeaxanthin, and canthaxanthin. In embodiments, the algae pigment is chlorophyll. In embodiments, the algae pigment is astaxanthin. In embodiments, the algae pigment is zeaxanthin. In embodiments, the algae pigment is canthaxanthin.

In an aspect is provided a composition including a palmitoleic acid (C16-1) alkali salt and a palmitic acid (C16-0) alkali salt.

In an aspect is provided a composition including a crystal phase and a liquid phase, wherein the crystal phase includes a urea crystal and palmitic acid (C16-0), and the liquid phase includes palmitoleic acid (C16-1).

In an aspect is provided a composition including a crystal phase and a liquid phase, wherein the crystal phase includes a thiourea crystal and palmitic acid (C16-0), and the liquid phase includes palmitoleic acid (C16-1).

In an aspect is provided a composition including palmitoleic acid (C16-1) and azelaic acid. In embodiments, the composition including palmitoleic acid (C16-1) and

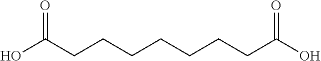 [SMILES: O=C(O)CCCCCCCC(=O)O]

(azelaic acid) further includes malonic acid, heptanoic acid, and/or 2-methylfumaric acid. In embodiments, the composition further includes

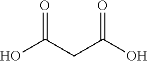

(malonic acid). In embodiments, the composition further includes

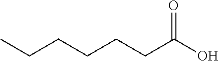

(heptanoic acid). In embodiments, the composition further includes

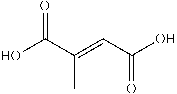

(2-methylfumaric acid). In embodiments, the composition further includes ozone.

In an aspect is provided a compound, or salt thereof, having the formula:

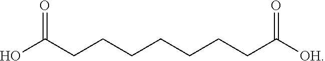 [SMILES: O=C(O)CCCCCCCC(=O)O]

In an aspect is provided a compound, or salt thereof, having the formula:

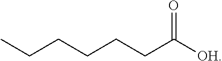

In an aspect is provided a composition including an algae polyester polymer and one or more of malonic acid, a 2-methylfumaric acid ester, and/or a terminal heptanoic acid ester.

In an aspect is provided a composition including an algae polyurethane and one or more of malonic acid, a 2-methylfumaric acid ester, or a terminal heptanoic acid ester. In embodiments, the one or more of malonic acid, a 2-methylfumaric acid ester, or a terminal heptanoic acid ester are present in a trace amount (e.g., detectable by mass spectrometry or high-performance liquid chromatography (HPLC)) to 1% of the total composition. In embodiments, the one or more of malonic acid, a 2-methylfumaric acid ester, or a terminal heptanoic acid ester are present in a trace amount (e.g., detectable by mass spectrometry or high-performance liquid chromatography (HPLC)) to 0.75% of the total composition. In embodiments, the one or more of malonic acid, a 2-methylfumaric acid ester, or a terminal heptanoic acid ester are present in a trace amount (e.g., detectable by mass spectrometry or high-performance liquid chromatography (HPLC)) to 0.5% of the total composition. In embodiments, the one or more of malonic acid, a 2-methylfumaric acid ester, or a terminal heptanoic acid ester are present in a trace amount (e.g., detectable by mass spectrometry or high-performance liquid chromatography (HPLC)) to 0.25% of the total composition. In embodiments, the one or more of malonic acid, a 2-methylfumaric acid ester, or a terminal heptanoic acid ester are present in a trace amount (e.g., detectable by mass spectrometry or high-performance liquid chromatography (HPLC)) to 0.1% of the total composition. In embodiments, the one or more of malonic acid, a 2-methylfumaric acid ester, or a terminal heptanoic acid ester are present in a trace amount (e.g., detectable by mass spectrometry or high-performance liquid chromatography (HPLC)) to about 1% of the total composition. In embodiments, the one or more of malonic acid, a 2-methylfumaric acid ester, or a terminal heptanoic acid ester are present in a trace amount (e.g., detectable by mass spectrometry or high-performance liquid chromatography (HPLC)) to about 0.75% of the total composition. In embodiments, the one or more of malonic acid, a 2-methylfumaric acid ester, or a terminal heptanoic acid ester are present in a trace amount (e.g., detectable by mass spectrometry or high-performance liquid chromatography (HPLC)) to about 0.5% of the total composition. In embodiments, the one or more of malonic acid, a 2-methylfumaric acid ester, or a terminal heptanoic acid ester are present in a trace amount (e.g., detectable by mass spectrometry or high-performance liquid chromatography (HPLC)) to about 0.25% of the total composition.

In embodiments, the one or more of malonic acid, a 2-methylfumaric acid ester, or a terminal heptanoic acid ester are present in a trace amount (e.g., detectable by mass spectrometry or high-performance liquid chromatography (HPLC)) to about 0.1% of the total composition.

In an aspect is provided a polyurethane composition that is a bio-based polyurethane composition, such that the origins of the bio-based (e.g., renewable components) are reflected in the ¹³C to ¹²C fractional content and/or the ¹⁴C to ¹²C carbon isotope ratio of the polyurethane.

In an aspect is provided a polyurethane composition having a ¹³C to ¹²C fractional content (δ¹³C) of about −60‰ to about −20‰. In embodiments, the ¹³C to ¹²C fractional content (δ¹³C) is about −50‰ to about −20‰. In embodiments, the ¹³C to ¹²C fractional content (δ¹³C) is about −40‰ to about −20‰. In embodiments, the ¹³C to ¹²C fractional content (δ¹³C) is about −34‰ to about −22‰. In embodiments, the ¹³C to ¹²C fractional content (δ¹³C) is about −31‰ to about −23‰. In embodiments, the ¹³C to ¹²C fractional content (δ¹³C) is about −31‰ to about −29‰. In embodiments, the ¹³C to ¹²C fractional content (δ¹³C) is about −29‰ to about −25‰. In embodiments, the ¹³C to ¹²C fractional content (δ¹³C) is about −25‰ to about −12‰. In embodiments, the ¹³C to ¹²C fractional content (δ¹³C) is about −25‰ to about −10‰. In embodiments, the ¹³C to ¹²C fractional content (δ¹³C) is about −23‰ to about −12‰. In embodiments, the ¹³C to ¹²C fractional content (δ¹³C) is about −20‰ to about −10‰. In embodiments, the ¹³C to ¹²C fractional content (δ¹³C) is −60‰ to −20‰. In embodiments, the ¹³C to ¹²C fractional content (δ¹³C) is −50‰ to −20‰. In embodiments, the ¹³C to ¹²C fractional content (δ¹³C) is −40‰ to −20‰. In embodiments, the ¹³C to ¹²C fractional content (δ¹³C) is −34‰ to −22‰. In embodiments, the ¹³C to ¹²C fractional content (δ¹³C) is −31‰ to −23‰. In embodiments, the ¹³C to ¹²C fractional content (δ¹³C) is −31‰ to −29‰. In embodiments, the ¹³C to ¹²C fractional content (δ¹³C) is −29‰ to −25‰. In embodiments, the ¹³C to ¹²C fractional content (δ¹³C) is −25‰ to −12‰. In embodiments, the ¹³C to ¹²C fractional content (δ¹³C) is −25‰ to −10‰. In embodiments, the ¹³C to ¹²C fractional content (δ¹³C) is −23‰ to −12‰. In embodiments, the ¹³C to ¹²C fractional content (δ¹³C) is −23‰ to −10‰. In embodiments, the ¹³C to ¹²C fractional content (δ¹³C) is less than −32‰. In embodiments, the ¹³C to ¹²C fractional content (δ¹³C) is less than −35‰. In embodiments, the ¹³C to ¹²C fractional content (δ¹³C) is less than −40‰. In embodiments, the ¹³C to ¹²C fractional content (δ¹³C) is less than −4‰. In embodiments, the ¹³C to ¹²C fractional content (δ¹³C) is less than −50‰. In embodiments, the ¹³C to ¹²C fractional content (δ¹³C) is less than −55‰. In embodiments, the ¹³C to ¹²C fractional content (δ¹³C) is less than −60‰.

In an aspect is provided an algae polyurethane composition produced by a method described herein, wherein the bio-based content of 30% or more, as calculated by the ¹⁴C/¹²C carbon isotope ratio of the polyurethane, according to ASTM D6866-12: Standard Test Methods for Determining the Bio-based Content of Solid, Liquid, and Gaseous Samples Using Radiocarbon Analysis Active Standard ASTM D6866.

In an aspect is provided an algae polyurethane composition produced by a method described herein, wherein the bio-based content of 50% or more, as calculated by the ¹⁴C/¹²C carbon isotope ratio of the polyurethane, according to ASTM D6866-12: Standard Test Methods for Determining the Bio-based Content of Solid, Liquid, and Gaseous Samples Using Radiocarbon Analysis Active Standard ASTM D6866.

In an aspect is provided an algae polyurethane composition produced by a method described herein, wherein the bio-based content of 70% or more, as calculated by the ¹⁴C/¹²C carbon isotope ratio of the polyurethane, according to ASTM D6866-12: Standard Test Methods for Determining the Bio-based Content of Solid, Liquid, and Gaseous Samples Using Radiocarbon Analysis Active Standard ASTM D6866.

In an aspect is provided an algae polyurethane composition produced by a method described herein, wherein the bio-based content of 90% or more, as calculated by the ¹⁴C/¹²C carbon isotope ratio of the polyurethane, according to ASTM D6866-12: Standard Test Methods for Determining the Bio-based Content of Solid, Liquid, and Gaseous Samples Using Radiocarbon Analysis Active Standard ASTM D6866.

In embodiments, the compound is a compound described herein (e.g., in the Compounds section, Examples Section, Methods Section, or in a claim, table, or figure).

### IV. Bio-Based Products

The compositions and methods provided herein find use in the synthesis and manufacture of polymers and products such as polyesters, polyurethanes, thermal polyurethanes, copolymers, and composites. In embodiments, the compositions and methods provided herein provide bio-based (e.g., renewable) components that are used to form polymers, such as polyesters.

In embodiments, the compositions and methods provided herein are used to synthesize a polyurethane. Such polyurethanes exhibit broad characteristics such as a range of hardness, high bearing load capacity, flexibility, and abrasion, impact, tear, and biological resistance. Such polyurethanes can be used in products such as foams for shoes, flip flops, car seats, mattresses, car tires, surf boards, insulation, rigid foams, skateboard and luggage wheels, paints, adhesives, coatings, fibers, textiles, clothing, and carpeting.

### V. Methods

In an aspect are provided methods of synthesis of a biobased polymer. The methods includes such methods as listed in Additional Embodiments 1-35. In embodiments, the methods include synthesizing one or more bio-based compositions for use in products such as foams.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### VI. Embodiments

Embodiment P1. A compound, or salt thereof, having a formula as described herein.

### VII. Additional Embodiments

Embodiment 1. A method of producing an algae free fatty acid composition comprising:
(a) contacting an algae fatty acid hydrolysate with a base thereby forming a soap composition;
(b) washing the soap composition with an organic solvent thereby removing one or more algae pigments to form a washed soap composition;
(c) contacting the washed soap composition with an acid thereby forming an algae free fatty acid composition.

Embodiment 2. The method of embodiment 1, wherein the algae free fatty acid composition comprises C16 free fatty acids.

Embodiment 3. The method of embodiment 2, wherein the C16 free fatty acids comprise palmitic (C16-0) free fatty acids and palmitoleic (C16-1) free fatty acids.

Embodiment 4. The method of one of embodiments 1 to 3, wherein the base is sodium hydroxide, potassium hydroxide, lithium hydroxide, or calcium hydroxide.

Embodiment 5. The method of one of embodiments 1 to 4, wherein the organic solvent is acetone, ether, or methyl tert-butyl ether.

Embodiment 6. The method of one of embodiments 1 to 5, wherein the one or more algae pigments are selected from the group consisting of a chlorophyll, a carotenoid, and a chlorophyll degradation product.

Embodiment 7. The method of one of embodiments 1 to 5, wherein the one or more algae pigments is selected from the group consisting of chlorophyll, astaxanthin, zeaxanthin, and canthaxanthin.

Embodiment 8. The method of one of embodiments 1 to 7, further comprising removing one or more saturated fatty acids from said algae fatty acid composition thereby forming an algae unsaturated fatty acid composition.

Embodiment 9. The method of embodiment 8, wherein the removing comprises contacting said algae fatty acid composition with methanol and applying temperature of about −15° C., thereby forming a crystalline fatty acid composition.

Embodiment 10. The method of embodiment 8, wherein the removing comprises contacting said algae fatty acid composition with urea at a temperature of about 4° C., thereby forming a crystalline fatty acid composition.

Embodiment 11. The method of one of embodiments 1 to 10, wherein the algae fatty acid hydrolysate is a microalgae fatty acid hydrolysate.

Embodiment 12. The method of one of embodiments 1 to 10, wherein the algae fatty acid hydrolysate is a Nannochloropsis sp. fatty acid hydrolysate, a Chlamydomonas sp. fatty acid hydrolysate, a Dunaliella sp. fatty acid hydrolysate, a Haematococcus sp. fatty acid hydrolysate, a Scenedesmus sp. fatty acid hydrolysate, a Diaphoreolis sp. fatty acid hydrolysate, or a Dunaliella sp. fatty acid hydrolysate.

Embodiment 13. The method of one of embodiments 1 to 10, wherein the algae fatty acid hydrolysate is a Nannochloropsis sp. fatty acid hydrolysate.

Embodiment 14. The method of one of embodiments 1 to 10, wherein the algae fatty acid hydrolysate is a C. reinhardtii fatty acid hydrolysate, D. satina fatty acid hydrolysate, H. pluvatis fatty acid hydrolysate, S. dimorphus fatty acid hydrolysate, D. viridis fatty acid hydrolysate, D. tertiolecta fatty acid hydrolysate, N. oculata fatty acid hydrolysate, or N. salina fatty acid hydrolysate.

Embodiment 15. The method of one of embodiments 1 to 10, wherein the algae fatty acid hydrolysate is a Cyanophyta fatty acid hydrolysate, a Prochlorophyta fatty acid hydrolysate, a Rhodophyta fatty acid hydrolysate, a Chlorophyta fatty acid hydrolysate, a Heterokontophyta fatty acid hydrolysate, a Tribophyta fatty acid hydrolysate, a Glaucophyta fatty acid hydrolysate, a Chlorarachniophyte fatty acid hydrolysate, a Euglenophyta fatty acid hydrolysate, a Euglenoid fatty acid hydrolysate, a Haptophyta fatty acid hydrolysate, a Chrysophyta fatty acid hydrolysate, a Cryptophyta fatty acid hydrolysate, a Cryptomonad fatty acid hydrolysate, a Dinophyta fatty acid hydrolysate, a Dinoflagellata fatty acid hydrolysate, a Prymnesiophyta fatty acid hydrolysate, a Bacillariophyta fatty acid hydrolysate, a Xanthophyta fatty acid hydrolysate, a Eustigmatophyta fatty acid hydrolysate, a Raphidophyta fatty acid hydrolysate, a Phaeophyta fatty acid hydrolysate, or a Phytoplankton fatty acid hydrolysate.

Embodiment 16. The method of one of embodiments 8 to 15, wherein the algae unsaturated fatty acid composition comprises palmitoleic acid (C16-1).

Embodiment 17. The method of embodiment 16, wherein the purity of the palmitoleic acid (C16-1) is from 85% to 90%.

Embodiment 18. The method of one of embodiments 8 to 17, further comprising contacting the algae unsaturated fatty acid composition with ozone followed by oxidation thereby forming an algae saturated dicarboxylic acid composition.

Embodiment 19. The method of embodiment 18, wherein the algae saturated dicarboxylic acid composition is an azelaic acid composition.

Embodiment 20. The method of embodiment 18 or embodiment 19, wherein the algae unsaturated fatty acid composition is contacted with ozone in an aqueous-organic solvent system.

Embodiment 21. The method of embodiment 20, wherein the aqueous-organic solvent system comprises aqueous acetonitrile.

Embodiment 22. The method of one of embodiments 18 to 21, wherein the oxidation is performed by the addition of aqueous sodium chlorite, followed by a quenching step with an aqueous reductant.

Embodiment 23. The method of one of embodiments 18 to 22, wherein the algae saturated dicarboxylic acid composition comprises heptanoic acid.

Embodiment 24. The method of embodiment 23, further comprising decarboxylating the heptanoic acid.

Embodiment 25. The method of embodiment 24, wherein the decarboxylating comprises catalytic hydrogenation or a light-dependent catalytic decarboxylation.

Embodiment 26. The method of embodiment 23, further comprising esterifying the heptanoic acid to heptanoyl methyl ester.

Embodiment 27. The method of one of embodiments 18 to 22, further comprising contacting the algae saturated dicarboxylic acid composition with a diol thereby forming an algae polyester polymer composition.

Embodiment 28. The method of embodiment 27, wherein the algae polyester polymer composition is formed using an esterification catalyst selected from the group consisting of a metal chloride, a metal oxide, a metal carboxylate, a metal alkoxide, and an organic acid.

Embodiment 29. The method of embodiment 28, wherein the algae saturated dicarboxylic acid composition is an algae azelaic acid composition and the diol is ethylene glycol.

Embodiment 30. The method of embodiment 28, wherein the algae saturated dicarboxylic acid composition is an algae oxalic acid composition, algae malonic acid composition, algae succinic acid composition, algae glutaric acid composition, algae adipic acid composition, algae 2,5-furandicarboxyic acid composition, algae pimelic acid composition, algae 3,3-dimethyl-1,2-cyclopropanedicarboxylic acid composition, algae suberic acid composition, algae azelaic acid composition, or algae sebacic acid composition.

Embodiment 31. The method of one of embodiments 27 to 30, wherein the diol is selected from the group consisting of ethylene glycol, 1,2 propanediol, 1,3-propanediol, glycerol, 1,3-butanediol, 1,4-butanediol, 2-methyl-1,3-propanediol, 2,3-butanediol, trimethylolpropane, 1,5-pentanediol, 1,6-hexanediol, 3-methyl-1,5-pentanediol, 1,7-heptanediol, 1,8-octanediol, 1,9-nonanediol, and 1,10-decanediol.

Embodiment 32. The method of one of embodiments 27 to 31, further comprising contacting said algae polyester polymer composition with a diisocyanate to form an algae polyurethane composition.

Embodiment 33. The method of embodiment 32, wherein the diisocyanate is selected from the group consisting of toluene diisocyanate (TDI), methylene diphenyl diisocyanate (MDI), hexamethylene diisocyanate (HDI), pentamethylene diisocyanate (PDI), 2,5-furandiisocyanate (FDI), heptamethylene diisocyanate (HPDI), hydrogenated MDI (H12MDI), and isophorone diisocyanate (IPDI).

Embodiment 34. The method of embodiment 32 or embodiment 33, wherein said algae polyurethane composition is formed using a catalyst selected from the group consisting of triethylenediamine, bis-(2-dimethylaminoethyl)-ether, N-methylmorpholine, N-ethylmorpholine, N,N,N′-trimethylisopropyl propylenediamine, dimethylcyclohexylamine, 1-methyl-4-dimethylaminoethylpiperazine, methoxypropyldimethylamine, N,N,N′,N′-tetramethyl-1,3-butanediamine, and dimethylethanolamine.

Embodiment 35. The method of embodiment 32 or embodiment 33, wherein the algae saturated dicarboxylic acid composition is an algae azelaic acid composition and said diol is an ethylene glycol.

Embodiment 36. A composition comprising an aqueous phase and an organic phase, wherein the aqueous phase comprises algae fatty acid alkali salts and the organic phase comprises one or more algae pigments.

Embodiment 37. The composition of embodiment 36, wherein the algae fatty acid alkali salt is an eicosapentaenoic acid (C20-5) alkali salt, a palmitoleic acid (C16-1) alkali salt, and/or a palmitic acid (C16-0) alkali salt.

Embodiment 38. The composition of embodiment 36 or embodiment 37, wherein the one or more algae pigments is selected from the group consisting of chlorophyll, astaxanthin, zeaxanthin, and canthaxanthin.

Embodiment 39. A composition comprising a palmitoleic acid (C16-1) alkali salt and a palmitic acid (C16-0) alkali salt.

Embodiment 40. A composition comprising a crystal phase and a liquid phase, wherein the crystal phase comprises a urea crystal and palmitic acid (C16-0), and the liquid phase comprises palmitoleic acid (C16-1).

Embodiment 41. A composition comprising palmitoleic acid (C16-1) and azelaic acid.

Embodiment 42. The composition of embodiment 41, further comprising malonic acid, heptanoic acid, and/or 2-methylfumaric acid.

Embodiment 43. The composition of embodiment 41 or embodiment 42, further comprising ozone.

Embodiment 44. A composition comprising an algae polyester polymer and one or more of malonic acid, a 2-methylfumaric acid ester, and/or a terminal heptanoic acid ester.

Embodiment 45. A composition comprising an algae polyurethane and one or more of malonic acid, a 2-methylfumaric acid ester, or a terminal heptanoic acid ester.

Embodiment 46. A polyurethane composition having a ¹³C to ¹²C fractional content (δ¹³C) of about −23‰ to about −12‰.

### EXAMPLES

### Example 1: Synthesis of Flexible Polyurethanes from a Microalgae Oil Waste Stream

Renewable polymers have become an important focus in next-generation materials, and algae biomass offers a low-impact feedstock source that can serve multiple uses. This study aims to develop a scalable methodology for production of microalgae-based polyols for polyurethane synthesis from waste oils derived from algae biomass. Following separation of omega-3 fatty acids from algae oil, residual oils can offer valuable building blocks for petrochemical replacements. However, unlike vegetable oils, algae oils contain multiple organic contaminants, including photosynthetic pigments and small organic molecules, that can complicate preparative chemistry. Here we convert and purify waste streams from omega-3 depleted Nannochloropsis salina algae oil, with major components consisting of palmitic and palmitoleic acid, into azelaic acid as a building block for flexible polyurethanes. Conversion of free fatty acid mixtures into a soft soap allows extraction of small organic contaminants through an acetone wash. Urea complexation provides isolated palmitoleic acid, which is subsequently ozonolyzed to produce azelaic acid. Bio-based polyester diols were then prepared via esterification to a provide polyol as a monomer for flexible polyurethane foams. This scalable process can be performed on oils from multiple algae species, offering a valuable monomer from a highly sustainable source.

To avoid fossil carbon use, renewable resources that offer the next generation of transportation fuels and chemical feedstocks have been studied in recent years^{2,3}. Although plant oils are known for low toxicity, renewability, and biodegradable properties^{4,5}, use of food crops poses other problems, including high arable land, water, and nutrient use. They also present a competition between biofuel feedstocks and food production⁶. For these reasons, algae biomass has experienced growing importance as a future potential source for producing sustainable energy and materials due to fast growth rate, flexible habitat preferences and substantial yield⁷. In addition, algae produce more unsaturated fatty acids compared to conventional vegetable oils⁸. Algae can be rapidly cultivated on large scales and need not consume non-arable land or fresh water⁹. One of the major challenges of algae production has been to identify strains with high growth rate, lipid content, and lipid productivity¹⁰. Identifying the highest and best use of algae oils will be important to ensure sustainability of the market, and omega-3 fatty acids from microalgae offer a high-value product and a growing adoption as nutraceuticals [doi: 10.1007/s10811-013-9983-9]. Nannochloropsis strains of green algae offer high titers of eicosapentaenoic acid (EPA), roughly 25% of total lipid content, and we reasoned that other high value applications could be explored with residual fatty acids. The polyurethane (PU) foam market is predicted to show continuous growth in coming years, from a USD 54.2 billion market in 2018 to over USD 79.8 billion predicted by 2023.¹ Without fundamental change, this rapid upward trend of global plastic demand is predicted to offset or reverse any decrease in petroleum demand from the use of renewable energies to 2050. [IEA. (2018, October 5). The future of petrochemicals. Retrieved from https://webstore.iea.org/the-future-of-petrochemicals.]

We previously established and developed a large-scale production system for cultivation of a variety of microalgae strains to produce biomass in both photobioreactors and open ponds¹¹, and algae oil has recently been applied both biofuel production and for polymeric material synthesis². The most abundant components in algae oil are triacylglycerides (TAGs), which can be easily hydrolyzed into saturated and unsaturated free fatty acids, including palmitic acid, palmitoleic acid, oleic acid, and linoleic acid. Unsaturations within these fatty acids can be converted into oxygenated functional groups (carboxyl or hydroxyl)¹², which can be valuable for a variety of chemical uses. The oxidative cleavage of olefins of unsaturated fatty acids is industrially carried out by ozonolysis¹³ for production of mono- and dicarboxylic acids, which play an important role in a variety of chemical applications such as polyesters, polyamides, plasticizers, and pharmaceuticals, among others^{14,15}. For instance, the ozonolysis of oleic acid has been shown to be a rapid source of azelaic acid and nonanoic acid^{14,15}, both of which have applications in polymer manufacturing.

As monomers with multiple hydroxyl group within their structures, polyols serve as precursors for production of polyurethane products such as flexible and rigid PU foams¹⁶. Recently, biobased polyols have emerged as renewable and sustainable monomers for the production of PU products. Previous publications showed the syntheses of several bio-polyols from palm, soya, corn, and castor^{17,18}, however vegetable oils offer comparatively pure source of TAGs. Algae biomass, by contrast, may be extracted to provide “green crude,” which contains significant photosynthetic pigments and other small organic molecules. In order to prepare homogenously pure feedstock for polymer synthesis, we have developed new methodologies for isolation and conversion of algae fatty acids into PU monomers.

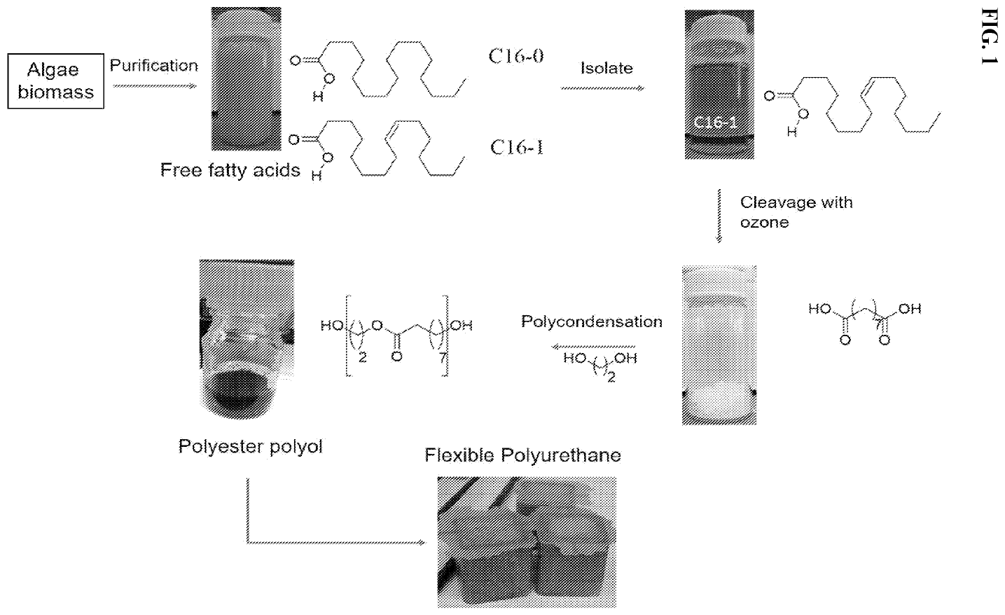

Here we have demonstrated a new approach to synthesize flexible PUs from omega-3 depleted algae biomass through the preparation of polyester polyols (FIG. 1). The procedure requires five stages: purification of fatty acids from omega-3 depleted algae oil; isolation of palmitoleic acid (C16-1) from free fatty acids; synthesis of azelaic acid (AA, C9-dicarboxylic acid) from C16-1; polycondensation of ethylene glycol and azelaic acid for polyester polyol synthesis; and polymerization with methylenediphenyl diisocyanate (MDI).

Materials and Methods.

Materials. Nannochloropsis salina was obtained from the National Center for Marine Algae and Microbiota, Maine, USA. Chemical reagents were purchased from Fisher Chemical, Macron Fine Chemicals, Sigma Aldrich, Acros, Fluka, Alfa Aesar Chemicals or TCI. All chemicals were regent grade and used without further purification. Analytical grade solvents such as acetone, hexane, and methanol were purchased from Fisher Chemical and used as received. Deuterated NMR solvents such as chloroform-d, DMSO-d6 were purchased from Cambridge Isotope Laboratories.

Equipment. Gas chromatography mass spectrometry (GC-MS) was run on an Agilent 7890A GC system connected to a 5975C VL MSD quadrupole MS (EI). Samples were separated on a 60 m DB23 Agilent GCMS column using helium as carrier gas and a gradient of 110° C. to 200° C. at 15° C.min⁻¹, followed by 20 minutes at 200° C. ¹H NMR spectra was recorded on a JOEL ECA 500 or a Varian VX500 spectrometer equipped with an Xsens Cold probe. ATR-FTIR was performed on a Perkin Elmer Spectrum RXI equipped with a ZnSe 1 mm ATR cell. 18 scans were taken at a 1 cm⁻¹ resolution. Gel permeation chromatography (GPC) was performed on Agilent GPC/SEC system, the samples were run in chloroform at 45° C. using a refractive index detector and analyzed against polystyrene standard. Fluorescence spectra were recorded at room temperature on a Thermo Scientific Varioskan LUX at the excitation wavelength of 350 nm. Differential Scanning calorimetry (DSC) was measured on Perkin-Elmer in a temperature range of −50 to 600° C. under Ar at flow rate of 20 mL min⁻¹ with a heating rate of 10 K min⁻¹. All data are referred to the second heating cycle. Ozone is produced from the Triogen LAB2B Ozone generator.

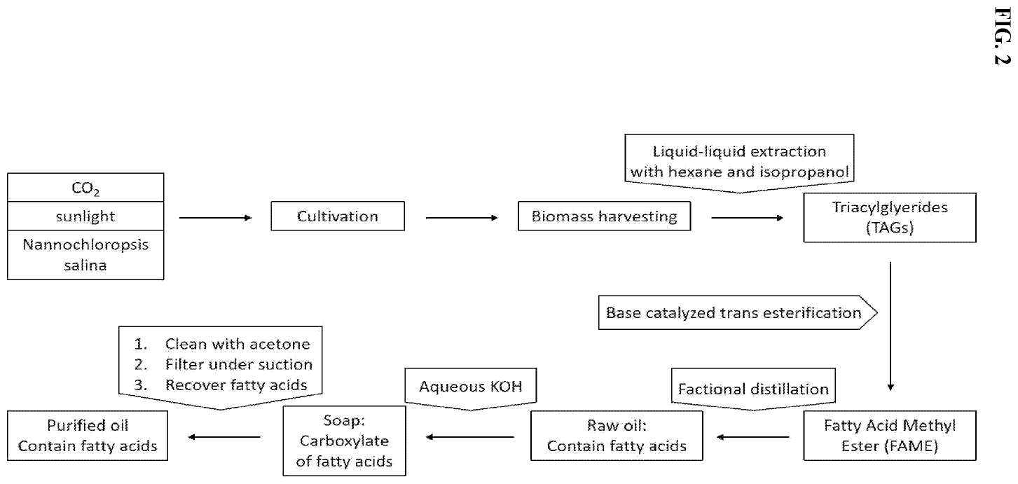

Step 1: Purification of fatty acids from biomass. Nannochloropsis salina¹⁹ was chosen as a strain for outdoor growth of microalgae biomass due to its robust growth and ability to accumulate high concentrations of polyunsaturated fatty acids¹¹. The procedure for algae culturing and harvesting of biomass has been described in our previous publication⁷. The harvested algae paste was collected and dried by centrifugation and storing at −20° C. and the triacylglycerides (TAGs) were extracted from using hexane and isopropanol using a liquid-liquid extraction technique²⁰. After a process of TAG hydrolysis, omega-3 fatty acids were isolated using fractional distillation to remove low boiling point distillates²¹, providing a mixture of saturated and monounsaturated fatty acids partially contaminated with photosynthetic pigments other small molecules. To remove non-fatty acid contaminants, saponification was carried out with 3 equivalents of (2N) aqueous KOH. The collected soft soap was washed with acetone several times obtain the purified soap. The fatty acids were recovered by acidification with 6N aqueous HCl (FIG. 2)

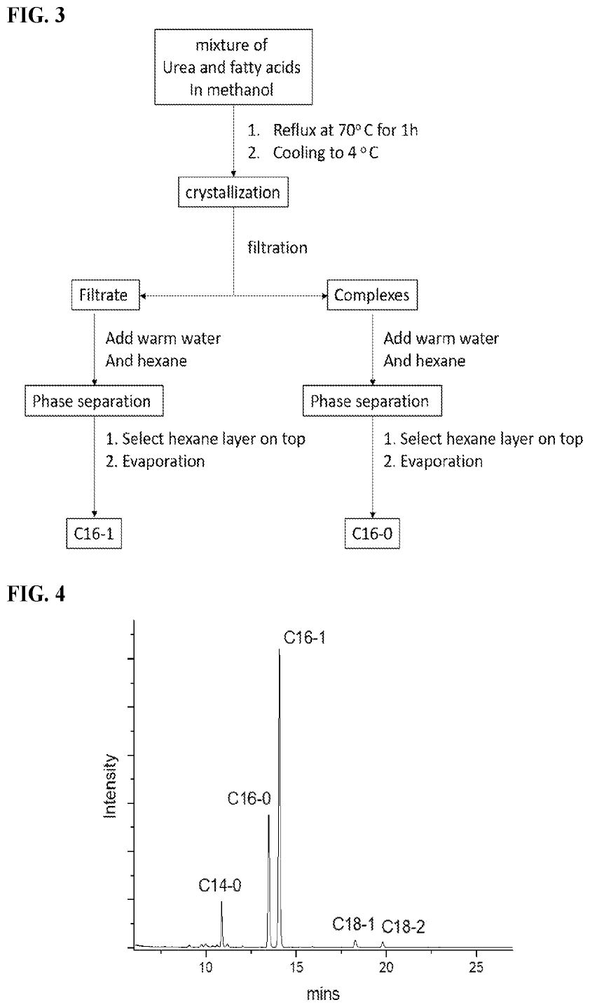

Step 2: Isolation mono-unsaturated fatty acid C16-1. Palmitoleic acid was isolated from a mixture of FFAs using a urea complexation method under optimal conditions (FIG. 3)^{22,23}. The free fatty acids (100 g) were mixed with 123 g urea in 770 mL methanol, then heated at 70° C. until the mixture became a homogeneous solution. The resulting mixture was slowly cooled to room temperature for 30 minutes before storing overnight at 4° C. for crystal formation. The crystals were separated from liquid by filtration under vacuum. Methanol was removed from the filtrate with a rotary evaporation, which was then washed with warm water (70° C.) and extracted with an equal volume of hexane. The hexane layer containing mono-unsaturated fatty acid was dried with anhydrous sodium sulfate before solvent removal by rotary evaporation to obtain pure palmitoleic acid.

Step 3: Oxidative cleavage of C16-1. Azelaic acid was synthesized by oxidative cleavage of mono-unsaturated fatty acid C16-1 with ozone as shown in Scheme 1. The procedure is similar to the process of synthesis of azelaic acid from oleic acid^{13-15,24}, except that a quench reaction was accomplished by sodium chlorite (NaClO₂)²⁵. C16-1 (20 g, 0.07 mol, 1 equiv) was dissolved in a mixture of 150 mL acetonitrile and 15 mL H₂O. The solution was cooled to 0° C. in ice bath and treated with ozone until the reaction complete, as confirmed by TLC. Once the ozonolysis completed, a 157 mL aqueous solution of 2M sodium chlorite (35.54 g, 0.31 mol, 4 equiv) was added dropwise into the cold reaction with the temperature controlled at 0° C. The reaction mixture turned yellow upon sodium chlorite addition. After standing overnight at room temperature, the mixture was reduced by slow addition of aqueous 2M sodium bisulfate (166 mL, 34.6 g, 0.33 mol, 4 equiv) under controlled temperature of 0° C. Once completed, the solution turned colorless and clarified, and the mixture was stirred for 10 minutes. Ethyl acetate (100 mL) added, and the two layers were separated. The organic phase, with azelaic acid and heptanoic acid, was dried by rotary evaporator to obtain a white paste product, which was diluted with hexane and extracted with hot water. Upon cooling the aqueous phase, azelaic acid (AA) formed as white crystals, which were filtered, washed several times with cold water, and dried. The hexane layer containing heptanoic acid was dried over Na₂SO₄, filtered, and concentrated as an oily liquid.

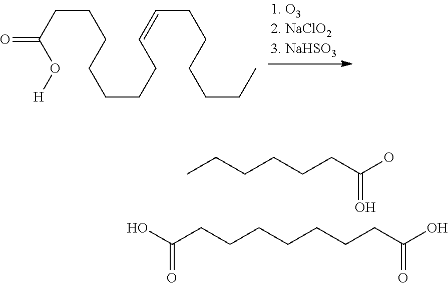

Scheme 1. Ozonolysis of Palmitoleic Acid

Step 4: Polycondensation of ethylene glycol and azelaic acid for polyester polyol synthesis. The reaction procedure for polyol synthesis was adapted from a literature report²⁶. To a 100 mL 3-neck flask, 17.8 g of azelaic acid and 8.7 g of ethylene glycol were combined. One neck was fitted with a gas inlet to allow dry nitrogen to flow through at a fixed flow rate to around 80 mL/min. The central neck was fitted with a thermometer for temperature verification. To the right neck was attached to a Dean-Stark apparatus to collect the water released by the esterification reaction. The apparatus was heated to 140° C. using a heating mantle with stirring to facilitate melting of the azelaic acid. At this point, 10 μL of dibutyltin dilaurate was added and the temperature gradually increased to 200° C. over the course of an hour. The reaction was allowed to proceed for a further 14 hours.

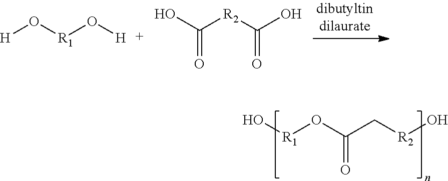

Scheme 2. Synthesis of Polyester Polyols

The polyols were characterized by OH number and acid number according to ASTM E1899 and D664, respectively using a Mettler Toledo G20S autotitrator with a non-aqueous electrode. For the OH number titrations, four replicates between 0.1 and 0.3 grams of polyol were reacted with p-toluene sulfonyl isocyanate (TSI) to form the carbamate, which was subsequently titrated with a standardized solution of 0.1 M tetrabutylammonium hydroxide in acetonitrile. The acid number titrations were performed in duplicate. 1 g of sample was diluted in a 50:49:1 solution of toluene, isopropanol, and water, then titrated with a standardized solution of 0.1 M KOH in isopropanol.

Step 5: Polyurethane polymerization with methylenediphenyl diisocyanate (MDI). A stainless steel mold with three 1″ cube slots was used to fabricate the foam samples. The mold was heated in an oven to 50° C. to ensure that the exothermic urethane reaction is sustained. Mold release (Stoner 5236) was applied by lightly spraying on to the mold sidewalls to ensure ease of demold. Polyols were heated to 50° C. to liquefy and reduce viscosity. All other components were used at room temperature. Polyol, catalyst, surfactant, water and isocyanate components were weighed into a cup and mixed with a DAC 600.1 Flacktek speed mixer at 2000 rpm for 17 seconds. The cube mold was placed on a balance. Each cube was hand poured from the cup into the mold to ensure consistent mass across cubes. The mold was then sealed and cured in an oven for 1 hour at 50° C., and then cooled to room temperature before demolding the cubes.

This study characterized four physical properties of the polyester polyurethane material: density, hardness, hysteresis, and peak force. The mass of each foam cube was measured on an analytical balance with an accuracy of plus or minus 0.01 grams. Density was determined by dividing the mass by the mold volume for each 1 inch cube. Hardness was measured by pressing a digital shore A durometer, made by FstDgte, into the center of each cube according to ASTM method D2240. The reported hardness is an average of the durometer measurements from all six faces of each cube.

Hysteresis and peak force were calculated using an AFG 2500N compression tester by MecMesin, with a MultiTest-dV sample stage. The test method was a compression of 50% of the original height of each cube, at a speed of 100 mm per minute. This instrument output a curve displaying each data point of force versus height of displacement. Energy loss was calculated as the integral under the curve for the compression, minus the integral under the curve for decompression. Percent hysteresis was calculated dividing the energy loss by the energy in. The peak force was measured on the 10th cycle of compression, in order to illustrate load-bearing capacity of the material.

Results and Discussion.

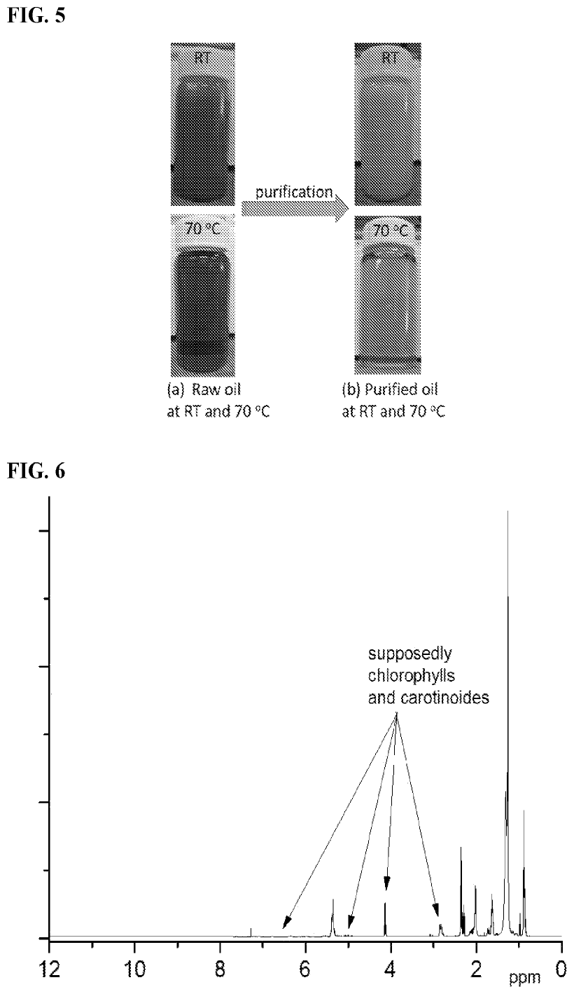

We chose the Nannochloropsis salina as a strain for growing algae in large scale because of its established high production of EPA and our ability to grow this strain for high biomass content^{27,11}. Following distillation to remove the low boiling point fractions from the omega-3 fatty acids, the composition of the unsaturated and saturated fatty acid mixture was identified by GC-MS by converting free fatty acids into fatty acid methyl ester. As shown in FIG. 4, these peaks were identified using retention time and mass spectral matching from the NIST reference library. The composition of the fatty acids is listed in FIG. 4: 8.3% of myristic acid (C14-0), 28.9% of palmitic acid (C16-0), 58.7% of palmitoleic acid (C16-1), 2.3% of oleic acid (C18-1), and 1.8% of linoleic acid (C18-2). The major constituents are palmitoleic acid (C16-1) and palmitic acid (C16-0. Importantly, the high percentage of palmitoleic acid is advantageous for isolation as the only monounsaturated fatty acid. Due to the presence of palmitic acid with melting point of 62.9° C., the raw oil is solid at room temperature that quickly melted to liquid when it reached temperature of 70° C. ((a) in FIG. 5).

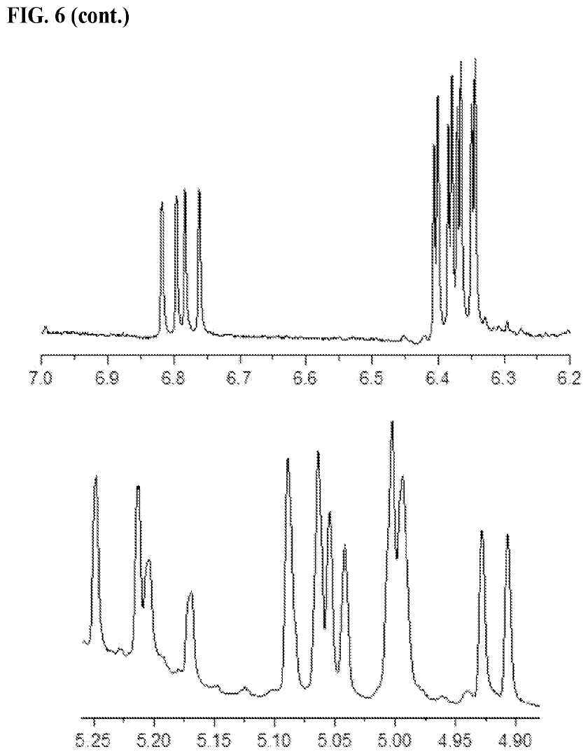

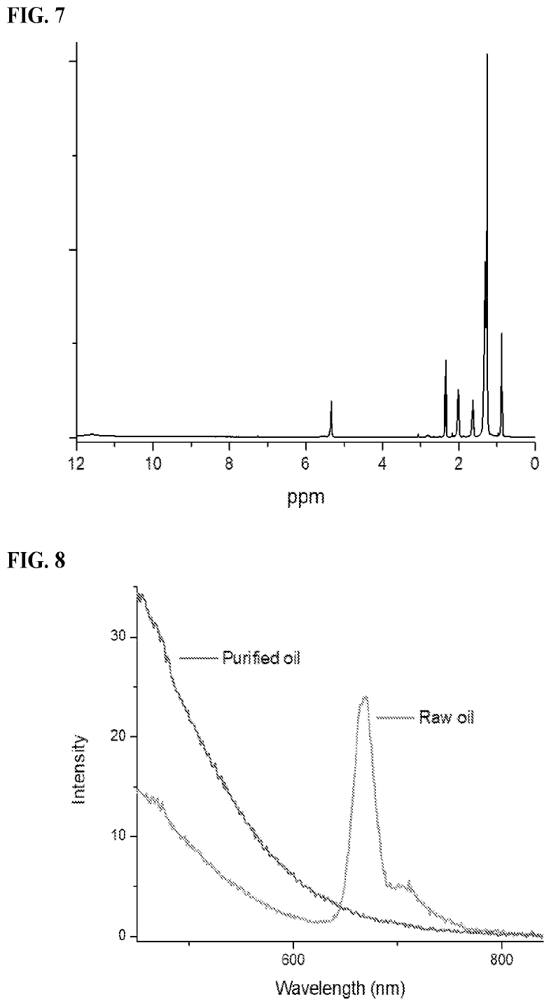

However, there are some possible photosynthesis pigments, such as chlorophyll fragments and carotenoids, contaminating the fatty acid fraction. The presence of these contaminants is evidenced by ¹H-NMR (FIG. 6) and reported in other publications^{2,28-30}. Most carotenoids and chlorophyll components are insoluble in water but freely soluble in organic solvents such as acetone, diethyl ether, tetrahydrofuran and chloroform^{31,32}. The presence of these pigments with conjugated pi systems may decrease the oxidative cleavage reaction efficiency, therefore the removal of chlorophylls and carotenoids is key process in the production of azelaic acid. There are a number of studies related to elimination of chlorophylls and carotenoids that include physical absorption, oxidation treatment, phosphoric acid degumming, precipitation, and bleaching³³⁻³⁷. However these are not practical for large scale applications. Recently, Li et al., showed a two-step process that includes bleaching combined with saponification to remove chlorophylls from oil, however a large proportion of oil was also lost³⁸. In this study, we found a practical pathway not only to improve the purity of oil but also to obtain high yield with a simple, cost-effective purification. The technique of saponification forms carboxylate salts of free fatty acids. After this, the obtained soap was washed and filtered several times with acetone until the observed filtrate turned from an orange to colorless solution, indicating that a removal of the pigments was complete. After elimination of pigments, the fatty acids were collected by acidification with aqueous hydrochloric acid. Depending on the types of bases used in the soap preparation, they can create two classes. Potassium hydroxide forms a soft soap, and sodium hydroxide forms a hard soap. Soft soap is in liquid form and thus more amenable to liquid extraction with acetone. Hard soap from sodium hydroxide requires more solvent and time to remove pigments due to the solid, waxy state. Treatment of washed soft soap with aqueous hydrochloric acid recovers a mixture of fatty acids with a yield of around 85%. As shown in FIG. 7, the ¹H NMR of purified oil indicated that impurity from pigments were eliminated. The presence of photosynthetic pigment can be accurately detected by fluorescence measurement down to parts per million^{39,40}. As shown in FIG. 8, raw oil displayed a strong emission peak at 668 nm, indicative of π-π* transition in photosynthetic pigments^{39,40} while the purified oil exhibited no emission peak. These color differences between raw oil ((a) in FIG. 5) and purified oil ((b) in FIG. 5) can also be clearly identified with the naked eye.

In the next step, we tried to find a suitable and practical method for palmitoleic acid C16-1 isolation from the mixture fatty acids in purified oil. In recent years, a variety of reported methods for separation of saturated and unsaturated fatty acids have been published, such as urea inclusion complexation^{22,23}, nanoporous membranes^{41,42} ion-liquid solvent extraction⁴³, molecular distillation⁴⁴, chromatography⁴⁵, supercritical fluid extraction⁴⁶, and lipase concentration⁴⁷. In many of the above-mentioned methods, urea complexation has proven to be a favorable technique for large-scale isolation of mono-unsaturated fatty acids due to its high separation capacity and simple process. Urea and thiourea are well known to form crystalline complexes with hydrocarbons, saturated fatty acids and other straight-chain molecules⁴⁸. This is made possible by a crystalline tunnel structure formed by urea that creates an inclusion site for linear compounds when packed densely with the guest molecule⁴⁹. Therefore, the presence of long, straight chain molecules of palmitic acid and other saturated fatty acids from mixtures is crystallize with urea in hexagonal structures. In contrast, the presence of cis-double bond in unsaturated fatty acids results in a kinked molecular structure⁵⁰, and consequently they cannot enter the hexagonal crystal channel and thus remain in the solvent. The fatty acids were converted to fatty acid methyl ester for GC-MS analysis and identification. Table 1 summarizes the compositions of the initial fatty acid mixture, as well as those of the palmitic and palmitoleic acid after separation by urea complexation.

TABLE 1
Fatty acid components and contents
in samples analyzed by GC-MS.

| | Samples | | |
|---|---|---|---|
| | Initial fatty | Isolated palmitoleic | Isolated palmitic |
| | acids | acid | acid |
| Fatty | Percentage of | C16-1 | C16-0 |
| acids | fatty acid | Percentage of fatty | Percentage of fatty |
| contents | methyl ester | acid methyl ester | acid methyl ester |
| C14-0 | 8.3 | 3.4 | 4.9 |
| C16-0 | 28.9 | 8 | 85.9 |
| C16-1 | 58.7 | 86 | 8.8 |
| C18-1 | 2.3 | 1.8 | 0.4 |
| C18-2 | 1.8 | 0.8 | 0 |

Note:
C14-0: “14” is the number of carbon atoms in the fatty acids molecule, while “0” is the number of carbon-carbon double bonds.

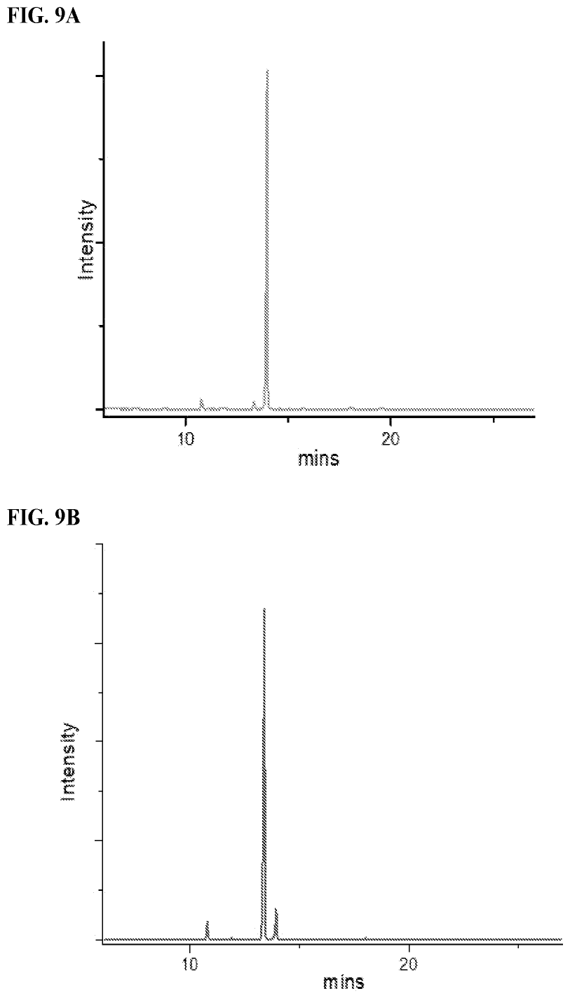

As can be seen from Table 1, a palmitoleic acid content of 86% with a yield of 80% was obtained from the liquid phase of urea complexation while the palmitic acid content of 85.9% was recovered from the solid phase. Importantly, most of the saturated fatty acid (palmitic acid (C16-0)) was removed from the unsaturated fatty acid (palmitoleic acid (C16-1)). There remains 8% of C16-0 in isolated C16-1 as the some of the saturated fatty acids do not bind with urea during crystallization⁵¹. The images of isolated palmitoleic acid (C16-1) and palmitic acid (C16-0) are also shown in FIGS. 9A-9B. The palmitic acid is a white solid that melts at 62.9° C., while the palmitoleic acid is yellow liquid at room temperature due to its melting point of −0.1° C.

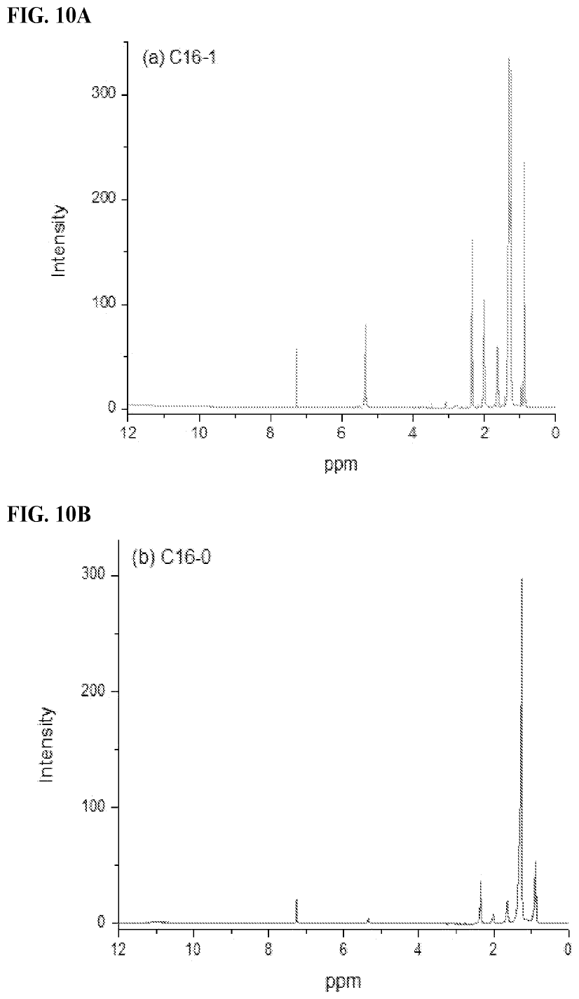

FIGS. 10A-10B display the ¹H NMR spectra of the palmitoleic acid C16-1 and palmitic acid C16-0. The signal at δ 0.71-1.11 corresponds to the terminal alkyl methyl, while the four peaks between δ 1.15-2.46 are assigned to the methylene groups nearest to the double bond and carboxyl group. The palmitoleic acid C16-1 (FIG. 10A) is identified by the high intensity signal at δ 5.24-5.50 belong to double bond. In contrast, there is a very low intensity signal at δ 5.22-5.44 in the ¹H NMR spectrum of palmitic acid C16-0 (FIG. 10B), indicative of the presence of small amount of C16-1. This finding is in agreement with the result of GC-MS (as shown in FIGS. 9A-9B).

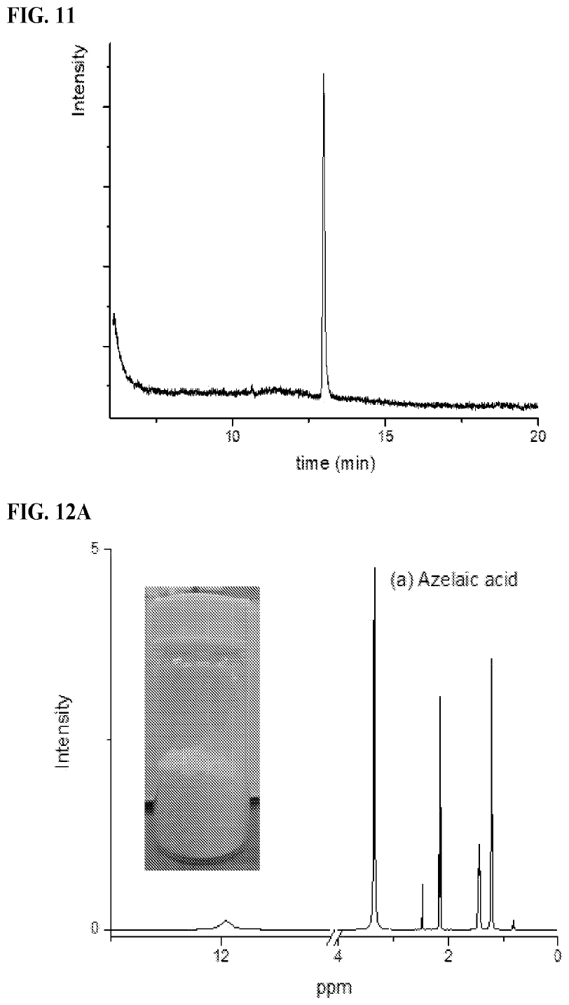

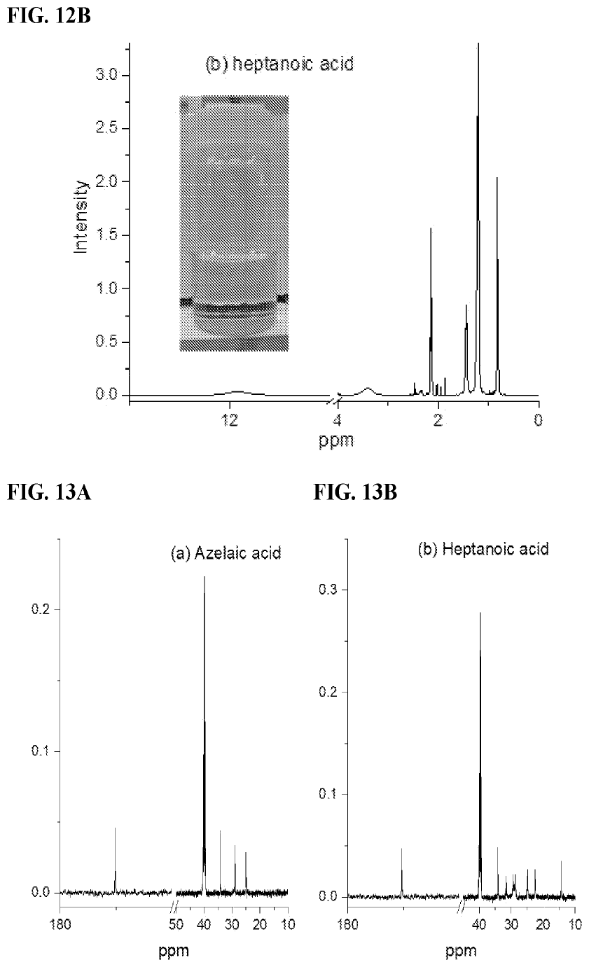

The next step is the process for production of azelaic acid through oxidative cleavage of palmitoleic acid (C16-1). Cleavage with ozone is well known to offer excellent selectivity, a simple procedure and the absence of toxic waste products from oxidants such as nitric acid, permanganate, or dichromate¹³. The three-step mechanism for ozonolysis of mono-unsaturated fatty acid has been reported in other publications^{13,14}. Due to a green and sustainable oxidizing agent, ozone is preferred as a safe alternative to other oxidants and catalysts^{13,52}. It is important to note that a continuous flow process with ozone has been developed for industrial applications, which is able to scale to tons of product per day⁵². Despite the advantages of ozonolysis, the process can suffer from sub-optimal yields, reported at around 70% for oleic acid^{13,15} Published procedures to date report azelaic acid yield of only 20%, although the ozonolysis of oleic acid has been optimized through high temperatures of up to 150° C. for 2 h¹⁵. Most published ozonolysis procedures quench the intermediate ozonide using 30-60% aqueous H₂O₂^{15,53}, initially producing aldehyde and carboxylic acid as products⁵³, that can result in low yields of mono- and dicarboxylic acid products^{15,54}. Therefore, to optimize the quench step, a variety of methods were investigated with the goal of complete conversion to carboxylic acid. The combination ozonolysis with oxidation using phosphotungstic acid or tungstic acid and quaternary ammonium salts were found to produce azelaic acid at around 70%⁵⁵⁻⁵⁷. The obtained yield of azelaic acid was also improved to 70% when combination ozonolysis with metal oxidation catalyst such as Mo-, V-, Mn-, Co-, Fe-, and Pb-oxides^{58,59}. Ackman, et al., used in situ formed performic acid from H₂O₂ and formic acid incorporation in methanol with oleic acid. Although this approach increased yield of azelaic acid up to 95%²⁴, it is only suitable for small laboratory scales due to safety concerns. In this case, instead of quenching with oxidative cleavage once completing ozonolysis, solvent was first removed under vacuum, followed by addition of H₂O₂ and HCOOH²⁴. As the formed ozonides or peroxides are potentially explosive, further workup with the presence of ozonides or proxides in the reaction mixture raised safety concerns. Recently, a mild one-pot ozonolysis oxidation process of olefins to synthesize carboxylic acids with the yields of up to 98% has been reported²⁵. This process, which employs sodium chlorite as an oxidant, is reported as a scalable procedure, safely converting over 20 kg of an alkene starting material in a high yield and purity²⁵. Therefore, we chose ozonolysis in combination with oxidative cleavage using sodium chlorite to prepare azelaic acid from palimitoleic acid C16-1. Azelaic acid was extracted with hot water from hexane solution of oxonolysis, followed by recrystallization. This ozonolysis-oxidation procedure obtained azelaic acid at 80-85%. The product was analyzed by GC-MS (FIG. 11) and ¹H and ¹³C NMR (FIG. 12A and FIG. 13A). HR-ESI-MS calcd. for azelaic acid—C₉H16O₄[M-H]⁻: 187.22, found 187.25.

Due to the relatively high boiling point of azelaic acid (286° C.), analysis was performed by converting azelaic acid into the dimethyl ester and analyzing by GC-MS. The dimethyl azelate was identified by matching mass spectral data to the NIST library database. As shown in FIG. 11, the retention time of the dimethyl azelate appeared at 13.16 minutes. The mass spectra of this observed peak was characterized with a cluster of fragmentation patterns and ions, which match well with the mass spectra reference library of dimethyl azelate. The identification of synthesized azelaic acid was also carried out by ¹H NMR, as shown in FIGS. 12A-12B. The signals at δ 1.0-1.8 ppm was assigned to methyl and methylene groups of azelaic acid, a triplet peak at δ 2.2 ppm correspond to C—H_{aliphatic} protons near the carboxylic group, and a broad signal around δ 12 is from carboxylic acid proton. The two peaks between δ 2.4 and 3.3 ppm belong to DMSO and water, respectively. The heptanoic acid product was also produced at over 85%, as analyzed by ¹H and ¹³C NMR (FIG. 12B and FIG. 13B). HR-ESI-MS calcd. for heptanoic acid—C₇H₁₄O₂[M-H]⁻: 129.18, found 129.23.

TABLE 2
Properties of polyester polyol.

| Polyol properties | Photosynthetic azelaic polyol |
|---|---|
| OH number, mg KOH/g | 109 ± 2 |
| Acid number, mg KOH/g | 0.28 ± 0.02 |
| GPC analysis - M_{w}, gmol⁻¹ | 18860 |
| (weight average molecular weight) | |
| GPC analysis - Mₙ, gmol⁻¹ | 5740 |
| (number average molecular | |
| weight) | |
| GPC analysis - polydispersity | 3.3 |
| index (PDI) | |

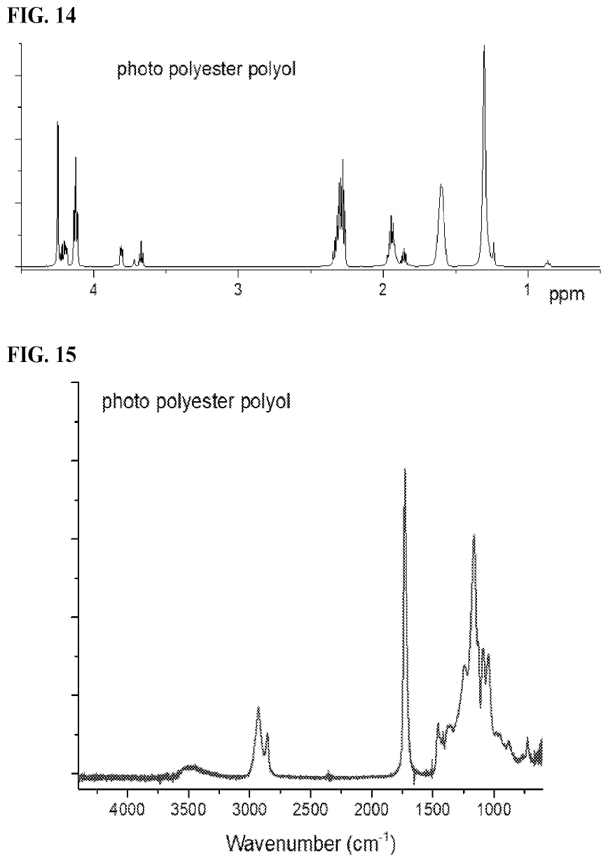

Polycondensation catalyzed by dibutyl dilaurate afforded a linear long chain polyester with a number average molecular weight and a weight average molecular weight of 5740 and 18860, respectively. Acid number of the polyester polyol in the range of 1-3 indicates near completeness of the reaction. The structure of polyester polyol are characterized by ¹H NMR spectroscopy (FIG. 14) and FT-IR (FIG. 15). The NMR result indicates the existence of both terminal and ester groups in the obtained polyol sample.

The spectrum displayed characteristic polyester polyol peaks at 3500 cm⁻¹ and 1730 cm⁻¹, showing OH and C═O stretching from the hydroxyl and ester carbonyl groups, respectively. The double peaks at 2927 and 2852 cm⁻¹ were consistent with C—H stretching from hydrocarbons, and the large peak at 1160 cm⁻¹ in the fingerprint region was also identified as ester C—O stretching. The broader OH peak in the algae-based polyol maybe because of a combination of lower hydroxyl number and/or residual acid content.

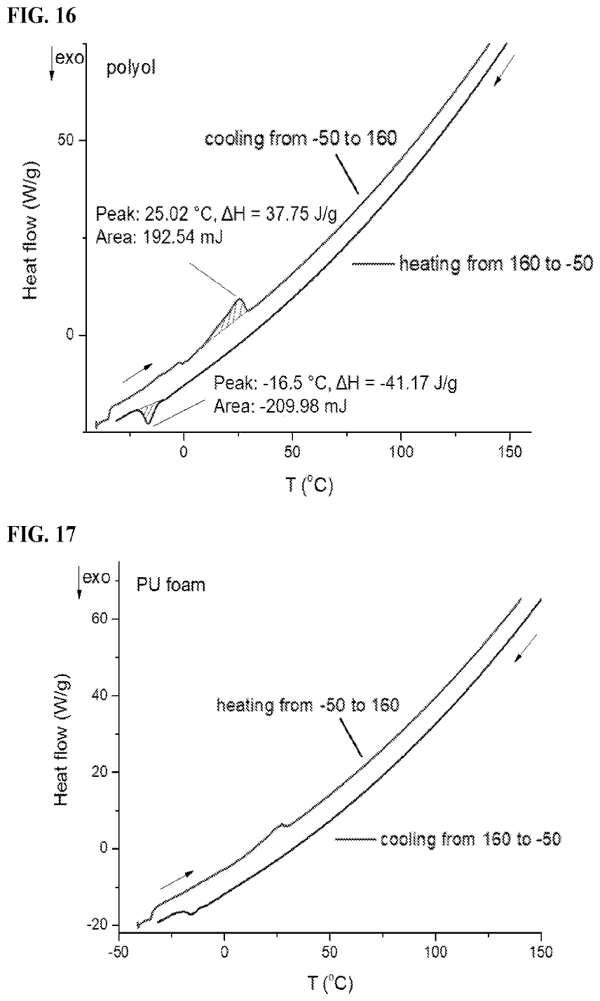

As illustrated in FIG. 16, the obtained polyol shows a melting point of 25° C. and glass transition temperature (Tg) within the range of −25 to −16.5° C. A broad and weak endothermic above Tg is due to the crystallization of a short chain in the polyol structure. Polyol exhibits a Tg below room temperature, a characteristic property indicates its elastomer behavior. A high mobility of short chains in the polyol results in the elasticity at temperature above the Tg⁶⁰.

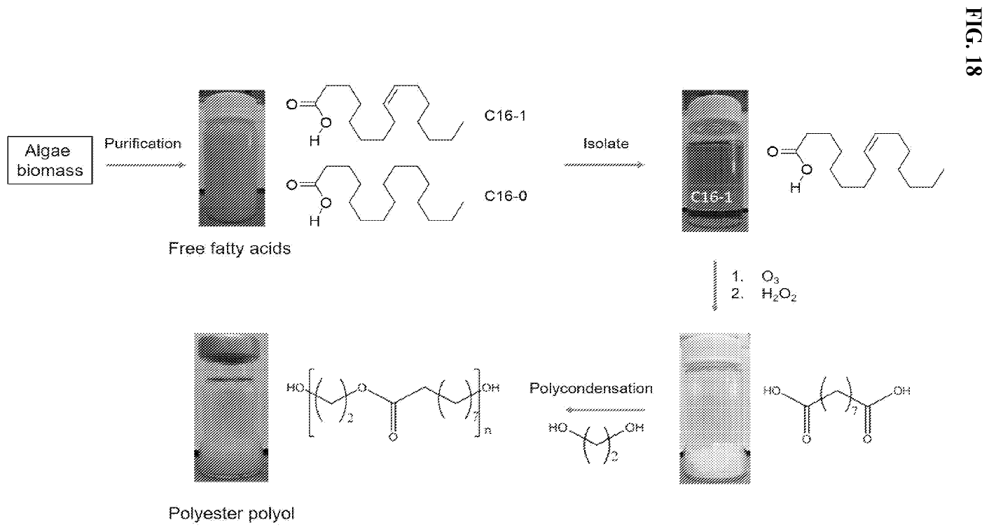

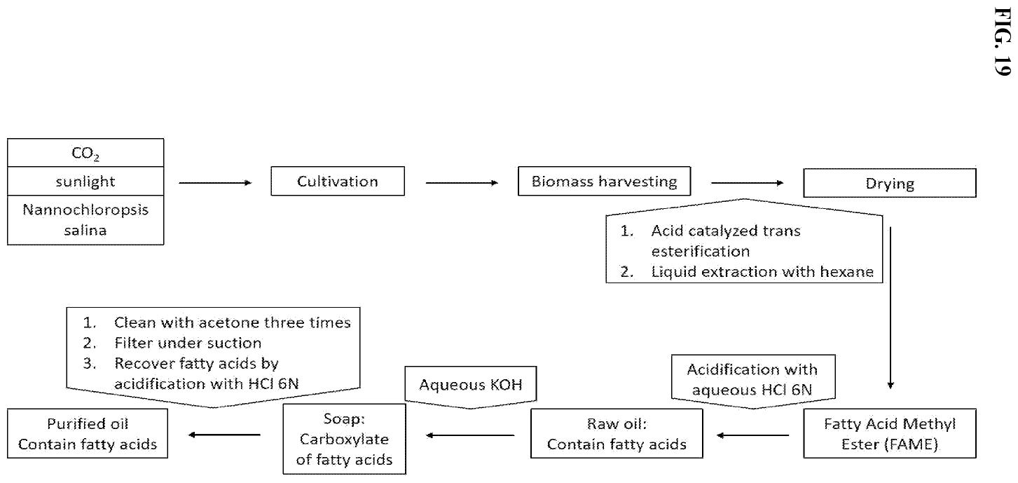

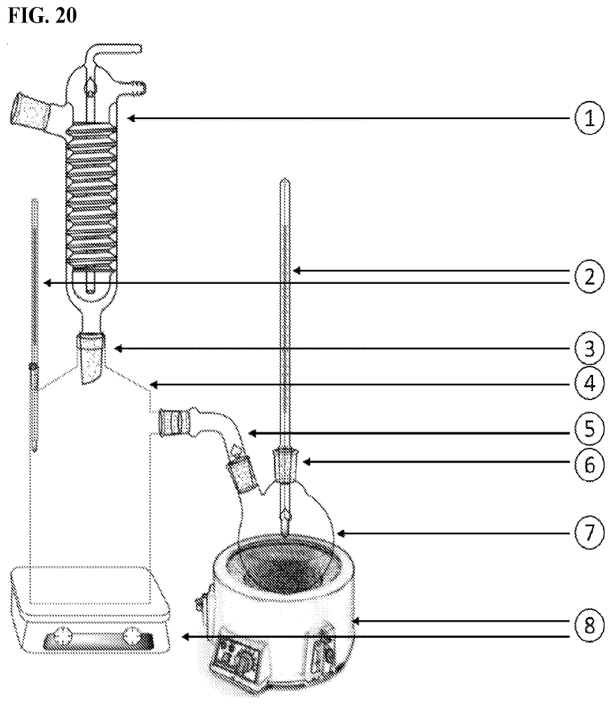

DSC curve of PU foam is presented in FIG. 17. The curve shows a glass transition temperature of 26.5° C. in a heating scan of PU foam. PU foam is 3D network structure which is formed by the link between the chain mobility of polyol—soft segment and MDI—hard segment. The PU matrix itself is highly cross-linked⁶¹. Therefore, the flexible of polyol chains lead to the good elasticity of PU foam at room temperature.

TABLE 3
Azelaic Polyol Foam Cube Properties.

| | Avg. | Avg. | Avg. | Avg. Peak |
|---|---|---|---|---|
| | Density | Hardness | Hysteresis | Force |
| Formula | (kg/m³) | (Shore A) | (%) | (N) |
| Photosynthetic PU foam | 297 ± 4 | 30 ± 3 | 51 ± 3 | 217 ± 17 |

The properties of algae-based cubes are shown in Table 3. Mechanical property is strongly depend on the degree of crosslinking and network structure of PU foam. The PU algae foams had high shore hardness, indicating the good connection in the PU foam between the soft segment of the algae polyol and the hard segment of MDI. The algae polyol is likely more crystalline, resulting in superior mechanical properties. This is corroborated by the fact that the algae polyol has high purity azelaic acid (GC-MS) and reacted faster when mixed with the isocyanate component. The hysteresis and peak force values trend well with shore hardness, and the more rigid algae cubes demonstrate lower energy loss and higher peak force as is expected.

In summary, azelaic acid was successfully prepared from an algae oil waste stream and converted into a flexible polyurethane. This study indicates that the algae-sourced azelaic acid has the potential to support material production of polyester polyols, a precursor for polyurethane synthesis, thereby valorizing a waste stream from omega-3 fatty acid production. We plan to optimize and scale these procedures to enable large scale production of azelaic acid. The exploration and utilization of algae biomass to prepare high value products offers tools to sustainably transition from petrochemicals to renewable chemical feedstocks.

### REFERENCES FOR EXAMPLE 1

(1) Austin, A.; Hicks, D. A. A review of the global PU industry 2016 and outlook for 2017. PU Magazine 2017, 14, 1-15. (2) Roesle, P.; Stempfle, F.; Hess, S. K.; Zimmerer, J.; Bártulos, C. R.; Eckert, B. L. A.; Kroth, P. G.; Mecking, S. Synthetic Polyester from Algae Oil. Angew. Chem. Int. Ed. 2014, 53, 6800-6804. (3) Meier, M. A. R.; Metzger, J. O.; Schubert, U. S. Plant oil renewable resources as green alternatives in polymer science. Chem. Soc. Rev. 2007, 36, 1788-1802. (4) Singh, D.; Bhoi, R.; Ganesh, A.; Mahajani, S. Synthesis of Biodiesel from Vegetable Oil Using Supported Metal Oxide Catalysts. Energy Fuels 2014, 28, 2743-2753. (5) Chaudhari, A. B.; Tatiya, P. D.; Hedaoo, R. K.; Kulkarni, R. D.; Gite, V. V. Polyurethane Prepared from Neem Oil Polyesteramides for Self-Healing Anticorrosive Coatings. Ind. Eng. Chem. Res. 2013, 52, 10189-10197. (6) Herrmann, R.; Jumbe, C.; Bruentrup, M.; EvansOsabuohien. Competition between biofuel feedstock and food production: Empirical evidence from sugarcane outgrower settings in Malawi. Biomass Bioenerg 2018, 114, 100-111. (7) Blatti, J. L.; Burkart, M. D. Releasing Stored Solar Energy within Pond Scum: Biodiesel from Algal Lipids. J. Chem. Educ. 2012, 89, 239-242. (8) Waghmare, A.; Patil, S.; LeBlanc, J. G.; Sonawane, S.; S. Arya, S. Comparative assessment of algal oil with other vegetable oils for deep frying. Algal Res. 2018, 31, 99-106. (9) Slade, R.; Bauen, A. Micro-algae cultivation for biofuels: Cost, energy balance, environmental impacts and future prospects. Biomass Bioenerg 2013, 53, 29-38. (10) Sara, M.; Brar, S. K.; Blais, J. F. Lipid production by Yarrowia lipolytica grown on biodiesel-derived crude glycerol: optimization of growth parameters and their effects on the fermentation efficiency. RSC Adv. 2016, 6, 90547-90558. (11) Schoepp, N. G.; L. Stewart, R.; Sun, V.; Quigley, A. J.; Mendola, D.; P. Mayfield, S.; D. Burkart, M. System and method for research-scale outdoor production of microalgae and cyanobacteria. Bioresour. Technol. 2014, 166, 273-281. (12) Kerenkan, A. E.; Béland, F.; Do, T.-O. Chemically catalyzed oxidative cleavage of unsaturated fatty acids and their derivatives into valuable products for industrial applications: a review and perspective. Catal. Sci. Technol. 2016, 6, 971-988. (13) Kockritz, A.; Martin, A. Synthesis of azelaic acid from vegetable oil-based feedstocks. Eur. J. Lipid sci. Technol. 2011, 113, 83-91. (14) Benessere, V.; Cucciolito, M. E.; Santis, A. D.; Serio, M. D.; Esposito, R.; Ruffo, F.; Turco, R. Sustainable Process for Production of Azelaic Acid Through Oxidative Cleavage of Oleic Acid. J Am. Oil. Chem. Soc. 2015, 92, 1701-1707. (15) Kadhum, A. A. H.; Wasmi, B. A.; Mohamad, A. B.; Al-Amiery, A. A.; Takriff, M. S. Preparation, characterization, and theoretical studies of azelaic acid derived from oleic acid by use of a novel ozonolysis method. Res. Chem. Intermed. 2012, 38, 659-668. (16) Gómez-Jiménez-Aberasturi, O.; Ochoa-Gómez, J. R. New approaches to producing polyols from biomass. J. Chem. Technol. Biotechnol. 2017, 92, 705-711. (17) Gu, R.; Konar, S.; Sain, M. Preparation and Characterization of Sustainable Polyurethane Foams from Soybean Oils. J. Am. Oil. Chem. Soc. 2012, 89, 2103-2111. (18) Petrovic, Z. S.; Wan, X.; Bilic, O.; Zlatanic, A.; Hong, J.; Javni, I.; Ionescu, M.; Milic, J.; Degruson, D. Polyols and Polyurethanes from Crude Algal Oil. J Am. Oil. Chem. Soc 2013, 90, 1073-1078. (19) Barrera, D. J.; Mayfield, S. P. High-value Recombinant Protein Production in Microalgae. Handbook of Microalgal Culture: Applied Phycology and Biotechnology 2013, 27, 532-544. (20) Schoepp, N. G.; Wong, W.; Mayfield, S. P.; Burkart, M. D. Bulk solvent extraction of biomass slurries using a lipid trap. RSC Adv. 2015, 5, 57038-57044. (21) Nam, H.; Choi, J.; Capareda, S. C. Comparative study of vacuum and fractional distillation using pyrolytic microalgae (Nannochloropsis oculata) bio-oil. Algal Res. 2016, 17, 87-96. (22) Hayes, D. G.; Bengtsson, Y. C.; Alstine, J. M. V.; Setterwall, F. Urea Complexation for the Rapid, Ecologically Responsible Fractionation of Fatty Acids from Seed Oil. J. Am. Oil Chem. Soc. 1998, 75, 1403-1409. (23) Bin Jiang; Yan Liu; Zhang, L.; Yongli Sun; Liu, Y.; Liu, X. Study on the Concentration of Unsaturated Fatty Acid Methyl Esters by Urea Complexation. J Chem. Soc. Pak. 2014, 36, 1013-1020. (24) Ackman, R. G.; Retson, M. E.; Gallay, L. R.; Vandenheuvel, F. A. Ozonolysis Of Unsaturated Fatty Acids I. Ozonolysis Of Oleic Acid. Can. J Chem. 1961, 39, 1956-1963. (25) Cochran, B. M. One-Pot Oxidative Cleavage of Olefins to Synthesize Carboxylic Acids by a Telescoped Ozonolysis-Oxidation Process. Synlett 2016, 27, 245-248. (26) Noor, N. M.; Sendijarevic, A.; Sendijarevic, V.; Sendijarevic, I.; Ismail, T. N. M. T.; Noor, M. A. M.; Kian, Y. S.; Hassan, H. A. Comparison of Adipic Versus Renewable Azelaic Acid Polyester Polyols as Building Blocks in Soft Thermoplastic Polyurethanes. J. Am. Oil. Chem. Soc. 2016, 93, 1529-1540. (27) Sukenik, A.; Carmeli, Y.; Berner, T. Regulation Of Fatty Acid Composition By Irradiance Level In The Eustigmatophyte Nannochloropsis Sp. J. Phycol. 1989, 25, 686-692. (28) Najdanova, M.; Siehl, H. U.; Berger, S. A comparison of the 1H-, 13C-, and 15N-NMR data of chlorophyll a and chlorophyll b guided by quantum chemical calculation. J Phys Org Chem. 2018, 31, e3802. (29) Willows, R. D.; Li, Y.; Scheer, H.; Chen, M. Structure of Chlorophyll f Org. Lett. 2013, 15, 1588-1590. (30) LaFountain, A. M.; Pacheco, C.; Prum, R. O.; Frank, H. A. Nuclear magnetic resonance analysis of carotenoids from the burgundy plumage of the Pompadour Cotinga (Xipholena punicea). Arch. Biochem. Biophys. 2013, 539, 133-141. (31) Craft, N. E.; Soares, J. H. Relative solubility, stability, and absorptivity of lutein and .beta.-carotene in organic solvents. J. Agric. Food Chem. 1992, 40, 431-434. (32) Gerola, A. P.; Tsubone, T. M.; Santana, A.; Oliveira, H. P. M. d.; Hioka, N.; Caetano, W. Properties of Chlorophyll and Derivatives in Homogeneous and Microheterogeneous Systems. J. Phys. Chem. B. 2011, 115, 7364-7373. (33) Bahmaei, M.; Sabbaghian, E. s.; Farzadkish, E. Development of a method for chlorophyll removal from canola oil using mineral acids. J. Am. Oil Chem. Soc. 2005, 82, 679-684. (34) Ghazani, S. M.; Marangoni, A. G. Minor Components in Canola Oil and Effects of Refining on These Constituents: A Review. J Am. Oil Chem. Soc. 2013, 90, 923-932. (35) Sathish, A.; Sims, R. C. Biodiesel from mixed culture algae via a wet lipid extraction procedure. Bioresour. Technol. 2012, 118, 643-647. (36) Chen, L.; Liu, T.; Zhang, W.; Chen, X.; Wang, J. Biodiesel production from algae oil high in free fatty acids by two-step catalytic conversion. Bioresour. Technol. 2012, 111, 208-214. (37) Diosady, L. L. Chlorophyll Removal From Edible Oils. Int. J Appl. Sci. Eng. 2005, 3. (38) Li, T.; Xu, J.; Wu, H.; GuanghuaWang; Dai, S.; Fan, J.; He, H.; Xiang, W. A Saponification Method for Chlorophyll Removal from Microalgae Biomass as Oil Feedstock. Mar. Drugs 2016, 14. (39) Kleinegris, D. M. M.; Es, M. A. v.; Janssen, M.; Brandenburg, W. A.; Wijffels, R. H. Carotenoid fluorescence in Dunaliella salina. J. Appl. Phycol. 2010, 22, 645-649. (40) Gillbro, T.; Cogdell, R. J. Carotenoid fluorescence. Chem. Phys. Lett. 1989, 158, 312-316. (41) Gilmer, C. M.; Zvokel, C.; Vick, A.; Bowden, N. B. Separation of saturated fatty acids and fatty acid methyl esters with epoxy nanofiltration membranes. RSC Adv. 2017, 7, 55626-55632. (42) Gupta, A.; Bowden*, N. B. Separation of cis-Fatty Acids from Saturated and trans-Fatty Acids by Nanoporous Polydicyclopentadiene Membranes. ACS Appl. Mater. Interfaces 2013, 5, 924-933. (43) Li, X.; Zhang, X.; Yang, Q.; Bao, Z.; Ren, Q.; Zhang, Z.; Xing, H.; Yang, Y. Separation of Highly Unsaturated Fatty Acid Methyl Esters from Model Bio-oils with Ionic Liquid-cosolvent as Extractants. RSC Adv. 2016, 6, 60709-60716. (44) Jiang, S. T.; Shao, P.; Pan, L. J.; Zhao, Y. Y. Molecular Distillation for recovering Tocopherol and Fatty Acid Methyl Esters from Rapeseed Oil Deodoriser Distillate. Biosyst. Eng. 2006, 93, 383-391. (45) Momchilova, S. M.; Nikolova-Damyanova, B. M. Advances in Silver Ion Chromatography for the Analysis of Fatty Acids and Triacylglycerols—2001 to 2011. Anal. Sci. 2012, 28, 837-844. (46) NuriaRubio-Rodríguez; Diego, S. M. d.; Beltran, S.; Jaime, I.; Sanz, M. T.; Rovira, J. Supercritical fluid extraction of fish oil from fish by-products: A comparison with other extraction methods. J. Food Eng. 2012, 109, 238-248. (47) Eun, L. J.; Hyuk, C. Y.; Palanivel, G.; Hae-Soo, K. Concentration of n-3 polyunsaturated fatty acids in cholesterol-reduced cod-liver oil by lipases. Int. J. Food Sci. Tech. 2011, 46, 1462-1468. (48) Simth, A. E. The Crystal Structure of the Urea-Hydrocarbon Complexes. Acta Cryst. 1952, 5, 224. (49) Harris, K. D. M. Fundamental and Applied Aspects of Urea and Thiourea Inclusion Compounds. Supramolecular Chemistry 2007, 19, 47-53. (50) Strocchi, A.; Bonaga, G. Correlation between urea inclusion compounds and conformational structure of unsaturated C18 Fatty acid methyl esters. Chem. Phys. Lipids 1975, 15, 87-94. (51) Ratnayake, W. M. N.; Olsson, B.; Matthews, D.; Ackman, R. G. Preparation of Omega-3 PUFA Concentrates from Fish Oils via Urea Complexation. Eur. J. Lipid Sci. Technol. 1988, 90, 381-386. (52) Nobis, M.; Roberge, D. R. Mastering ozonolysis: production from laboratory to ton scale in continuous flow. Chim. Oggi—Chem. Today 2011, 29, 56-58. (53) Irfan, M.; Glasnov, T. N.; Kappe*, C. O. Continuous Flow Ozonolysis in a Laboratory Scale Reactor. Org. Lett. 2011, 13, 984-987. (54) Katrib, Y.; Martin, S. T.; Hung, H.-M.; Rudich, Y.; Zhang, H.; Slowik, J. G.; Davidovits, P.; Jayne, J. T.; Worsnop, D. R. Products and Mechanisms of Ozone Reactions with Oleic Acid for Aerosol Particles Having Core-Shell Morphologies. J. Phys. Chem. A 2 2004, 108, 6686-6695. (55) Shi, C.-W.; Chen, Y.-P.; Jia, R.-P. Preparation of azelaic acid by oxidation with hydrogen peroxide and ozone. Huaxue Shijie 2007, 48, 612-614. (56) Shi, C.-W.; Chen, Y.-P. Preparation of azelaic acid by oxidation with hydrogen peroxide and ozone. Xiandai Huagong 2006, 26, 285-287. (57) Shi, C.; Yan, Y.; Yin, W. Study on synthesis and reaction mechanism of azelaic acid by united oxidation. Huagong Keji 2009, 17, 16-19. (58) Z.-C., S.; Y.-G., Z.; W., E.; S.-M., H.; J.-J., G. Production of azelaic acid by the compound solvent-ozonization-catalytic oxidation process from oleic acid. Zhongguo Youzhi 2006, 31, 40-42. (59) Sun, Z.-C.; Wumanjiang, E.; Xu, T.-Y.; Zhang, Y.-G. Catalysts for oxidation of the ozonolysis product of oleic acid with oxygen to make azelaic acid. Yingyong Huaxue 2006, 23, 161-164. (60) Baharu, M. N.; Kadhum, A. A. H.; Al-Amiery, A. A.; Mohamad, A. B. Synthesis and characterization of polyesters derived from glycerol, azelaic acid, and succinic acid. Green Chem Lett Rev 2015, 8, 31-38. (61) Raftopoulos, K. N.; Pagacz, J.; Ozimek, J.; Koutsoumpis, S.; Michalowski, S.; Hebda, E.; Pielichowski, J.; Pielichowski, K. Molecular dynamics in polyurethane foams chemically reinforced with POSS. Polym. Bull. 2019, 76, 2887-2898.

### Example 2: Synthesis of Azelaic Acid from Algae Oil

Renewable polymers have become an important focus in next-generation materials, and algae biomass offers a low-impact source of feedstock. This study aims to develop a scalable methodology for production of algae-based polyols for polyurethane synthesis from algae biomass. Unlike vegetable oils, algae oils contain multiple photosynthetic pigments and small organic molecules that can complicate process chemistry. Here we convert and purify crude algae oil from Nannochloropsis salina to obtain mixture of fatty acids with major components consisting of palmitic and palmitoleic acid. Urea complexation offers isolated monounsaturated fatty acids, which are subsequently treated by ozonolysis to produce pure azelaic acid. Bio-based polyester diols were then prepared via esterification to provide polyol for flexible polyurethane foams. This scalable process can be performed on oils from multiple algae species, offering a robust supply of diacids for monomer preparation from among the lowest impact biomass species.

According to Plastic Insight (www.platicsinsight.com), Research Market (www.researchandmarkets.com), and PU magazine¹, the polyurethane (PU) foam market is predicted to show continuous growth in coming years, from a USD 54.2 billion market in 2018 to over USD 79.8 billion predicted by 2023. This rapid upward trend of global PU demand will raise the risks associated with petrochemicals, including increasing greenhouse gas emissions and use of diminishing supplies of fossil petroleum. To avoid problems posed by fossil carbon use, renewable resources that offer the next generation of transportation fuels and chemical feedstocks have been studied in recent years^{2,3}. Although plant oils are known for low toxicity, renewability, and biodegradable properties^{4,5}, use of food crops poses other problems, including high arable land, water, and nutrient use. They also present a competition between biofuel feedstocks and food production⁶.

For these reasons, algae biomass has experienced growing importance as a future potential source for producing sustainable energy and materials due to fast growth rate, flexible habitat preferences and substantial yield.⁸ In addition, algae produce more unsaturated fatty acids compared to conventional vegetable oils⁹. Algae can be rapidly cultivated on large scales and need not consume non-arable land or fresh water⁹. One of the major challenges of algae production has been to identify strains with high growth rate, lipid content, and lipid productivity¹⁰. Previously, we successfully established and developed a large-scale production system for cultivation of a variety of microalgae strains to produce biomass in both photobioractors and open ponds¹¹, and algae oil has recently been applied both biofuel production and for polymeric material synthesis². The most abundant components in algae oil are triacylglycerides (TAGs), which can be easily hydrolyzed into saturated and unsaturated free fatty acids, including palimitic acid, palmitoleic acid, oleic acid, and linoleic acid. Unsaturations within these fatty acids can be converted into oxygenated functional groups (carboxyl or hydroxyl)¹², which can be valuable for a variety of chemical uses. The oxidative cleavage of olefins of unsaturated fatty acids is industrially carried out by ozonolysis¹³ for production of mono- and dicarboxylic acids, which play an important role in a variety of chemical applications such as polyesters, polyamides, plasticizers, and pharmaceuticals, among others^{14,15}. For instance, the ozonolysis of oleic acid has been shown to be a rapid source of azelaic acid and nonanoic acid^{14,15}, both of which have applications in polymer manufacturing. As monomers with multiple hydroxyl group within their structures, polyols serve as precursors for production of polyurethane products such as flexible and rigid PU foams¹⁶. Recently, biobased polyols have emerged as renewable and sustainable monomers for the production of PU products. Previous publications showed the syntheses of several bio-polyols from palm, soya, corn, and castor^{17,18} however vegetable oils offer comparatively pure source of TAGs. Algae biomass, by contrast, may be extracted to provide “green crude,” which contains significant photosynthetic pigments and other small organic molecules. In order to prepare homogenously pure feedstocks for polymer synthesis, we have developed new methodologies for isolation and conversion of algae fatty acids into PU monomers.

Here we demonstrate a new approach to synthesize flexible PUs from algae biomass through the preparation of polyester polyols (FIG. 18). The procedure requires five stages: purification of fatty acids from bulk algae biomass; isolation of palmitoleic acid (C16-1) from free fatty acids; synthesis of azelaic acid (AA, C9-dicarboxylic acid) from C16-1; polycondensation of ethylene glycol and azelaic acid for polyester polyol synthesis; and polymerization with methylenediphenyl diisocyanate (MDI).

Materials and Methods.

Materials. Nannochloropsis salina was obtained from the National Center for Marine Algae and Microbiota, Maine, USA. Hydrogen peroxide (30%), hydrochloric acid (36.5-38%), sodium sulfate anhydrous, potassium hydroxide, urea were purchased from Fisher Chemical. All chemicals were regent grade and used without further purification. Analytical grade solvents such as acetone, hexane, and methanol were purchased from Fisher Chemical and used as received. Deuterated NMR solvents such as chloroform-d and DMSO-d6 were purchased from Cambridge Isotope Laboratories.

Equipment. Gas chromatography mass spectrometry (GC-MS) was run on an Agilent 7890A GC system connected to a 5975C VL MSD quadrupole MS (EI). Samples were separated on a 60 m DB23 Agilent GCMS column using helium as carrier gas and a gradient of 110° C. to 200° C. at 15° C.min⁻¹, followed by 20 minutes at 200° C. ¹H NMR spectra was recorded on a JOEL ECA 500 or a Varian VX500 spectrometer equipped with an Xsens Cold probe. Gel permeation chromatography (GPC) was performed on Agilent GPC/SEC system, the samples were run in chloroform at 45° C. using a refractive index detector and analyzed against polystyrene standard. Fluorescence spectra were recorded at room temperature on a Thermo Scientific Varioskan LUX at the excitation wavelength of 350 nm. Differential Scanning Calorimetry (DSC) was measured on Perkin-Elmer STA 6000 Simultaneous Thermal Analyzer in a temperature range of 25 to 160° C. under N₂ at flow rate of 20 mL min⁻¹ with a heating rate of 10 K min¹. Ozone is produced from the Triogen LAB2B Ozone generator.

Step 1: Purification of fatty acids from biomass. Nannochloropsis salina¹⁹ was chosen as a strain for outdoor growth of microalgae biomass due to its robust growth and ability to accumulate high concentrations of polyunsaturated fatty acids¹¹. The procedure for algae culturing and harvesting of biomass has been described in our previous publication⁸. The harvested algae paste was collected and dried by centrifugation and storing at −20° C. and the TAGs were extracted from using hexane and isopropanol using a liquid-liquid extraction technique²⁰. A raw oil containing mixture of free fatty acids was produced by base hydrolysis of purified TAGs to provide free fatty acids (FFAs), and a mixture of unsaturated and monounsaturated fatty acids were isolated using fractional distillation. Comparative study of vacuum and fractional distillation using pyrolytic microalgae (Nannochloropsis oculata) bio-oil. Algal research. 2016 Jul. 1; 17:87-96.] To remove photosynthetic pigments contaminating the obtained fatty acids, saponification was carried out with aqueous KOH. The collected soft soap was washed with acetone several times, then filtered under vacuum to obtain purified soft soap. The molar ratio between free fatty acids and KOH is one to three, and volume of deionized water is approximately 1.5 times the mass of KOH. The fatty acids were recovered by acidification of soft soap with aqueous HCl 6N (FIG. 19).

Step 2: Isolation mono-unsaturated fatty acid C16-1. The mono unsaturated fatty acid—C16-1 (palmitoleic acid) was isolated from a mixture of FFAs by urea complexation method under optimal conditions. Here urea:FFA (w/w) of 1.23 and MeOH:urea (v/w) of 6.25^{21,22}. The urea complexation procedure is shown as described in FIG. 3. The fatty acids (100 g) were mixed with 123 g urea in 770 mL methanol, then heated at 70° C. until the mixture became a homogeneous solution. The resulting mixture was slowly cooled to room temperature for 30 minutes before storing overnight at 4° C. for crystal formation. The crystals were separated from liquid by filtration under vacuum. Methanol was removed from the filtrate with a rotary evaporation, which was then washed with warm water (70° C.) and extracted with an equal volume of hexane. The hexane layer containing mono-unsaturated fatty acid was dried with anhydrous sodium sulfate before solvent removal by rotary evaporation to obtain pure palmitoleic acid.

Step 3: Oxidative cleavage of C16-1. Azelaic acid was synthesized by oxidative cleavage of mono-unsaturated fatty acid C16-1 with ozone as shown in Scheme 3. The procedure is similar to the process of synthesis of azelaic acid from oleic acid^{13-15,23}, except a quench reaction was accomplished by sodium chlorite (NaClO₂)²⁴. C16-1 (20 g, 0.07 mol, 1 equiv) was dissolved in a mixture of 150 mL acetonitrile and 15 mL H₂O. The solution was cooled to 0° C. in ice bath and treated with ozone until the reaction complete, as confirmed by TLC. Once the ozonolysis completed, a 157 mL aqueous solution of 2M sodium chlorite (35.54 g, 0.31 mol, 4 equiv) was added dropwise into the cold reaction with the temperature controlled at 0° C. The reaction mixture turns yellow upon sodium chlorite addition. After standing overnight at room temperature, the mixture was reduced by slow addition of aqueous 2M sodium bisulfite (166 mL, 34.6 g, 0.33 mol, 4 equiv) into the mixture under controlled temperature of 0° C. Once completed, the solution turned colorless and clarified, and the mixture was stirred for 10 minutes. Ethyl acetate (100 mL) added, and the two layers were separated. The organic phase, with azelaic acid and heptanoic acid, was dried by rotovap to obtain a white paste product, which was extracted with hot water and hexane. Upon cooling the aqueous phase, azelaic acid (AA) formed as white crystals, which were filtered, washed several times with cold water, and dried. The hexane layer containing heptanoic acid was then dried over Na₂SO₄, filtered, and concentrated as an oily liquid.

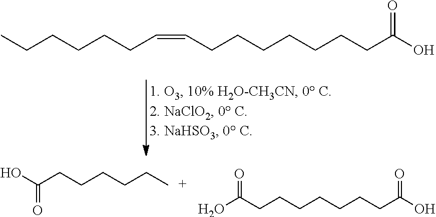

Scheme 3. Ozonolysis of Palmitoleic Acid

Step 4: Polycondensation of Ethylene Glycol and Azelaic Acid for Polyester Polyol Synthesis.

Step 5: Polyurethane Polymerization with Methylenediphenyl Diisocyanate (MDI).

Results and Discussion.

We chose the Nannochloropsis salina as a strain for growing algae in large scale because this strain is very effective in production of unsaturated fatty acids and demonstrated highly consistent growth^{11,25}. Following distillation, the composition of the unsaturated and saturated fatty acid mixture was identified by GC-MS by converting free fatty acids into fatty acid methyl ester. As shown in FIG. 4, these peaks were identified using retention time and mass spectral matching from reference library, while the relative percentage of each component calculated from the areas of the peaks to the total areas. The composition of the fatty acids are listed in FIG. 4: 8.3% of myristic acid (C14-0), 28.9% of palmitic acid (C16-0), 58.7% of palmitoleic acid (C16-1), 2.3% of oleic acid (C18-1), and 1.8% of linoleic acid (C18-2). The major constituents are palmitoleic acid (C16-1) and palmitic acid (C16-0), importantly, the high percentage of palmitoleic acid is advantageous and useful for the isolation of mono-unsaturated fatty acid. Due to the presence of palmitic acid with melting point of 62.9° C., the raw oil is solid at room temperature that quickly melted to liquid when reached temperature of 70° C. ((a) in FIG. 5).

However, there are some possible photosynthesis pigments such as chlorophyll fragments and carotenoids along with fatty acids that may cause raw oil to possess a dark brown color. The presence of chlorophylls and carotenoids are evidenced by ¹H-NMR (FIG. 6) and reported in multiple publications^{2,26-28.} Most carotenoids and chlorophyll are insoluble in water but freely soluble in organic solvents such as acetone, diethyl ether, tetrahydrofuran and chloroform^{29,30}. The presence of these pigments with conjugated pi systems may decrease the oxidative cleavage reaction efficiency; therefore, the removal of chlorophylls and carotenoids is key process in the production of azelaic acid. There are a number of studies related to elimination of chlorophylls and carotenoids that include physical absorption, oxidation treatment, phosphoric acid degumming, precipitation, and bleaching³¹⁻³⁵. However, these are not practical for large scale applications. Recently, Li et al., showed a two-step process that includes bleaching combined with saponification to remove chlorophylls from oil, however a large proportion of oil was also lost³⁶. In this study, we found a practical pathway not only to improve the purity of oil but also to obtain high yield with reasonable cost. The technique of saponification forms carboxylate salts of free fatty acids. After this, the obtained soap was washed and filtered several times with acetone until the observed filtrate turned from an orange to colorless solution, indicating that a removal of the pigments was complete. After elimination of pigments, the fatty acids were collected by acidification with aqueous hydrochloric acid. Depending on the types of bases used in the soap preparation, they create two classes. Potassium hydroxide forms a soft soap, and sodium hydroxide forms a hard soap. Soft soap is in liquid form and thus more amenable to acetone. Hard soap from sodium hydroxide requires more solvent and time to remove pigments due to the solid, waxy state. Treatment of washed soft soap with aqueous hydrochloric acid recovers a mixture of fatty acids with a yield of around 85%. As shown in FIG. 7, the ¹H NMR of purified oil indicated that all impurity from pigments were eliminated. The presence of photosynthetic pigment can be accurately detected by fluorescence. As shown in FIG. 8, raw oil displayed a strong emission peak at 668 nm, indicative of π-π* transition in photosynthetic pigments,^{37,38} while the purified oil exhibited no emission peak. These color differences between raw oil ((a) in FIG. 5) and purified oil ((b) in FIG. 5) can also be clearly identified with the naked eye.

In the next step, we tried to find a suitable and practical method palmitoleic acid C16-1 isolation from mixture fatty acids in purified oil. In recent years, a variety of reported methods for separation of saturated and unsaturated fatty acids have been published, such as urea inclusion complexation^{21,22}, nanoporous membranes^{39,40}, ion-liquid solvent extraction⁴¹, molecular distillation⁴², chromatography⁴³, supercritical fluid extraction⁴⁴, and lipase concentration⁴⁵. In the number of the above-mentioned methods, urea complexation has proven to be a favorable technique for large-scale isolation of mono-unsaturated fatty acids due to its high separation capacity and simple process. Theoretically, urea and thiourea are facile to form crystalline complexes with hydrocarbons, fatty acids and other straight chain molecules⁴⁶. Moreover, the tunnel structure formed by urea offers inclusion site for linear compounds solely when it is packed densely with a guest molecule⁴⁷. Therefore, the presence of long, straight chain molecules of palmitic acid from fatty acids mixtures is able to make urea crystallization in hexagonal structures. In contrast, the presence of the double bond results in the larger molecular size of palmitoleic acid⁴⁸, and consequently they cannot enter the hexagonal crystal channel and they remained in the organic solvent. The fatty acids were converted to fatty acid methyl ester for GC-MS analysis and identification. Table 4 summarizes the compositions of the initial fatty acid mixture, as well as those of the palimitic and palmitoleic acid after separation by urea complexation.

TABLE 4
Fatty acid components and contents
in samples analyzed by GC-MS

| | Samples | | |
|---|---|---|---|
| | Initial fatty | Isolated palmitoleic | Isolated palmitic |
| | acids | acid | acid |
| Fatty | Percentage of | C16-1 | C16-0 |
| acids | fatty acid | Percentage of fatty | Percentage of fatty |
| contents | methyl ester | acid methyl ester | acid methyl ester |
| C14-0 | 8.3 | 3.4 | 4.9 |
| C16-0 | 28.9 | 8 | 85.9 |
| C16-1 | 58.7 | 86 | 8.8 |
| C18-1 | 2.3 | 1.8 | 0.4 |
| C18-2 | 1.8 | 0.8 | 0 |

Note:
C14-0: “14” is the number of carbon atoms in the fatty acids molecule, while “0” is the number of carbon-carbon double bonds.

As can be seen from Table 4, the palmitoleic acid content of 86% with a yield of 80% was obtained from the liquid phase of urea complexation, while the palmitic acid content of 85.9% was recovered from the solid phase. Importantly, most of saturated fatty acid (palmitic acid (C16-0)) was removed from the unsaturated fatty acid (palmitoleic acid (C16-1)). There remains 8% of C16-0 in isolated C16-1, as the some of the saturated fatty acids do not bind with urea during crystallization⁴⁹. The images of isolated palmitoleic acid (C16-1) and palmitic acid (C16-0) are also shown in FIGS. 9A-9B. The palmitic acid is a white solid that melts at 62.9° C. while the palimitoleic acid is yellow liquid at room temperature due to its melting point of −0.1° C.

FIGS. 10A-10B displays the ¹H NMR spectra of the palmitoleic acid C16-1 and palmitic acid C16-0. The signal at δ 0.71-1.11 corresponds to the terminal alkyl methyl, while the four peaks between δ 1.15-2.46 are assigned to the methylene groups nearest to the double bond and carboxyl group. The palmitoleic acid C16-1 (FIG. 10A) is identified by the high intensity signal at δ 5.24-5.50 belong to double bond. In contrast, there is a very low intensity signal at δ 5.22-5.44 in the ¹H NMR spectrum of palmitic acid C16-0 (FIG. 10B), indicative of the presence of small amount of C16-1. This finding is in agreement with the result of GC-MS (as shown in FIGS. 9A-9B).

The next step is the process for production of azelaic acid through oxidative cleavage of palmitoleic acid (C16-1). Cleavage with ozone is well known to offer excellent selectivity, simple procedure and the absence of toxic waste products from oxidants such as nitric acid, permanganate, or dichromate¹³. The three-step mechanism for ozonolysis of mono-unsaturated fatty acid has been reported in other publications^{13,14}. Due to a green and sustainable oxidizing agent, ozone is preferred as a safe alternative to other oxidants and catalysts^{13,50}. It is important to note that a continuous flow process with ozone has been developed for industrial applications, which is able to produce on the ton scale of product per day⁵⁰. Despite the advantages of ozonolysis, the process can suffer from sub-optimal yields, reported at around 80% for oleic acid^{13,15}. Most published ozonolysis procedures quenched the intermediate ozonide by using 30-60% aqueous H₂O₂ ^{15,51}, initially producing aldehyde and carboxylic acid as products⁵¹, and can result in low yields of mono and di carboxylic acid products^{15,52}. Therefore, to optimize the quench step, a variety of methods were investigated with the goal of complete conversion to carboxylic acid. The combination ozonolysis with oxidation using phosphotungstic acid or tungstic acid and quaternary ammonium salts were found to produce azelaic acid at around 70%⁵³⁻⁵⁵. The obtained yield of azelaic acid were also improved to 70% when combination ozonolysis with some oxidation catalyst such as Mo-, V-, Mn-, Co-, Fe-, and Pb-containing oxides and tungstic acid^{56,57}. Ackman, et al., used in situ formed performic acid from H₂O₂ and formic acid incorporation in methanol with oleic acid. Although this approach increased yield of azelaic acid up to 95%²³, it is only suitable for small laboratory scales. Here, instead of quenching with oxidative cleavage once completing ozonolysis, solvent was first removed under vacuum, followed by addition of H₂O₂ and HCOOH²³. As the formed ozonides or peroxides are potentially explosive, further workup with the presence of ozonides or proxides in the reaction mixture raised safety concerns. Recently, a mild one-pot ozonolysis oxidation process of olefins to synthesize carboxylic acids with the yields of up to 98% have been reported²⁴. This process is scalable, safely converting over 20 kg of an alkene starting material in a high yield and purity²⁴. Therefore, we chose ozonolysis in combination with oxidative cleavage with sodium chlorite to prepare azelaic acid from palimitoleic acid C16-1. This ozonolysis-oxidation procedure obtained azelaic acid at 80-85%. The product was analyzed by GC-MS (FIG. 11) and ¹H NMR (FIG. 12A).

Due to the relatively high boiling point of azelaic acid (286° C.), analysis can be performed by converting azelaic acid into their dimethyl ester and analysis by GC-MS. The dimethyl azelate was primarily identified by matching mass spectral data to the NIST library database. As shown in FIG. 11, the retention time of the dimethyl azelate appeared at 13.16 minutes. The mass spectra of this observed peak was characterized with a cluster of fragmentation patterns and ions, which match well with the mass spectra reference library of dimethyl azelate. The identification of synthesized azelaic acid was also carried out by ¹H NMR, as shown in FIGS. 12A-12B. The signals at δ 1.0-1.8 was assigned to methyl and methylene groups of azelaic acid, a triplet peak at δ 2.2 correspond to C—H_{aliphatic} protons near the carboxylic group, and a broad signal around δ 12 is from carboxylic acid proton. The two peaks between δ 2.4 and 3.3 belong to DMSO and water, respectively. In summary, both GC-MS and ¹H NMR identified the formation of pure azelaic acid. Furthermore, the heptanoic acid product was also produced at over 85%, as analyzed by ¹H NMR (FIG. 12B).

In summary, azelaic acid was successfully prepared from algae biomass and converted into a flexible polyurethane. This study indicates that the synthesized azelaic acid has the potential to support material production of polyester polyols, a precursor for polyurethane synthesis. In next step, an optimization and study of the procedures for large scale production of azelaic acid will be a focus. The exploration and utilization of algae biomass to prepare azelaic acid offers a useful development toward sustainable polyurethanes.

### REFERENCES FOR EXAMPLE 2

(1) Austin, A.; Hicks, D. A. A review of the global PU industry 2016 and outlook for 2017. PU Magazine 2017, 14, 1-15. (2) Roesle, P.; Stempfle, F.; Hess, S. K.; Zimmerer, J.; Bártulos, C. R.; Eckert, B. L. A.; Kroth, P. G.; Mecking, S. Synthetic Polyester from Algae Oil. Angew. Chem. Int. Ed. 2014, 53, 6800-6804. (3) Meier, M. A. R.; Metzger, J. O.; Schubert, U. S. Plant oil renewable resources as green alternatives in polymer science. Chem. Soc. Rev. 2007, 36, 1788-1802. (4) Singh, D.; Bhoi, R.; Ganesh, A.; Mahajani, S. Synthesis of Biodiesel from Vegetable Oil Using Supported Metal Oxide Catalysts. Energy Fuels 2014, 28, 2743-2753. (5) Chaudhari, A. B.; Tatiya, P. D.; Hedaoo, R. K.; Kulkarni, R. D.; Gite, V. V. Polyurethane Prepared from Neem Oil Polyesteramides for Self-Healing Anticorrosive Coatings. Ind. Eng. Chem. Res. 2013, 52, 10189-10197. (6) Herrmann, R.; Jumbe, C.; Bruentrup, M.; EvansOsabuohien. Competition between biofuel feedstock and food production: Empirical evidence from sugarcane outgrower settings in Malawi. Biomass Bioenerg 2018, 114, 100-111. (7) Waghmare, A.; Patil, S.; LeBlanc, J. G.; Sonawane, S.; S. Arya, S. Comparative assessment of algal oil with other vegetable oils for deep frying. Algal Res. 2018, 31, 99-106. (8) Blatti, J. L.; Burkart, M. D. Releasing Stored Solar Energy within Pond Scum: Biodiesel from Algal Lipids. J. Chem. Educ. 2012, 89, 239-242. (9) Slade, R.; Bauen, A. Micro-algae cultivation for biofuels: Cost, energy balance, environmental impacts and future prospects. Biomass Bioenerg 2013, 53, 29-38. (10) Sara, M.; Brar, S. K.; Blais, J. F. Lipid production by Yarrowia lipolytica grown on biodiesel-derived crude glycerol: optimization of growth parameters and their effects on the fermentation efficiency. RSC Adv. 2016, 6, 90547-90558. (11) Schoepp, N. G.; L. Stewart, R.; Sun, V.; Quigley, A. J.; Mendola, D.; P. Mayfield, S.; D. Burkart, M. System and method for research-scale outdoor production of microalgae and cyanobacteria. Bioresour. Technol. 2014, 166, 273-281. (12) Kerenkan, A. E.; Béland, F.; Do, T.-O. Chemically catalyzed oxidative cleavage of unsaturated fatty acids and their derivatives into valuable products for industrial applications: a review and perspective. Catal. Sci. Technol. 2016, 6, 971-988. (13) Kockritz, A.; Martin, A. Synthesis of azelaic acid from vegetable oil-based feedstocks. Eur. J. Lipid Sci. Technol. 2011, 113, 83-91. (14) Benessere, V.; Cucciolito, M. E.; Santis, A. D.; Serio, M. D.; Esposito, R.; Ruffo, F.; Turco, R. Sustainable Process for Production of Azelaic Acid Through Oxidative Cleavage of Oleic Acid. J. Am. Oil. Chem. Soc. 2015, 92, 1701-1707. (15) Kadhum, A. A. H.; Wasmi, B. A.; Mohamad, A. B.; Al-Amiery, A. A.; Takriff, M. S. Preparation, characterization, and theoretical studies of azelaic acid derived from oleic acid by use of a novel ozonolysis method. Res. Chem. Intermed. 2012, 38, 659-668. (16) Gómez-Jiménez-Aberasturi, O.; Ochoa-Gómez, J. R. New approaches to producing polyols from biomass. J. Chem. Technol. Biotechnol. 2017, 92, 705-711. (17) Gu, R.; Konar, S.; Sain, M. Preparation and Characterization of Sustainable Polyurethane Foams from Soybean Oils. J Am. Oil. Chem. Soc. 2012, 89, 2103-2111. (18) Petrovic, Z. S.; Wan, X.; Bilic, O.; Zlatanic, A.; Hong, J.; Javni, I.; Ionescu, M.; Milic, J.; Degruson, D. Polyols and Polyurethanes from Crude Algal Oil. J Am. Oil. Chem. Soc 2013, 90, 1073-1078. (19) Barrera, D. J.; Mayfield, S. P. High-value Recombinant Protein Production in Microalgae. Handbook of Microalgal Culture: Applied Phycology and Biotechnology 2013, 27, 532-544. (20) Schoepp, N. G.; Wong, W.; Mayfield, S. P.; Burkart, M. D. Bulk solvent extraction of biomass slurries using a lipid trap. RSC Adv. 2015, 5, 57038-57044. (21) Hayes, D. G.; Bengtsson, Y. C.; Alstine, J. M. V.; Setterwall, F. Urea Complexation for the Rapid, Ecologically Responsible Fractionation of Fatty Acids from Seed Oil. J. Am. Oil Chem. Soc. 1998, 75, 1403-1409. (22) Bin Jiang; Yan Liu; Zhang, L.; Yongli Sun; Liu, Y.; Liu, X. Study on the Concentration of Unsaturated Fatty Acid Methyl Esters by Urea Complexation. J. Chem. Soc. Pak. 2014, 36, 1013-1020. (23) Ackman, R. G.; Retson, M. E.; Gallay, L. R.; Vandenheuvel, F. A. Ozonolysis Of Unsaturated Fatty Acids I. Ozonolysis Of Oleic Acid. Can. J Chem. 1961, 39, 1956-1963. (24) Cochran, B. M. One-Pot Oxidative Cleavage of Olefins to Synthesize Carboxylic Acids by a Telescoped Ozonolysis-Oxidation Process. Synlett 2016, 27, 245-248. (25) Sukenik, A.; Carmeli, Y.; Berner, T. Regulation Of Fatty Acid Composition By Irradiance Level In The Eustigmatophyte Nannochloropsis Sp. J. Phycol. 1989, 25, 686-692. (26) Najdanova, M.; Siehl, H. U.; Berger, S. A comparison of the 1H-, 13C-, and 15N-NMR data of chlorophyll a and chlorophyll b guided by quantum chemical calculation. J Phys Org Chem. 2018, 31, e3802. (27) Willows, R. D.; Li, Y.; Scheer, H.; Chen, M. Structure of Chlorophyll f Org. Lett. 2013, 15, 1588-1590. (28) LaFountain, A. M.; Pacheco, C.; Prum, R. O.; Frank, H. A. Nuclear magnetic resonance analysis of carotenoids from the burgundy plumage of the Pompadour Cotinga (Xipholena punicea). Arch. Biochem. Biophys. 2013, 539, 133-141. (29) Craft, N. E.; Soares, J. H. Relative solubility, stability, and absorptivity of lutein and .beta.-carotene in organic solvents. Agric. Food Chem. 1992, 40, 431-434. (30) Gerola, A. P.; Tsubone, T. M.; Santana, A.; Oliveira, H. P. M. d.; Hioka, N.; Caetano, W. Properties of Chlorophyll and Derivatives in Homogeneous and Microheterogeneous Systems. J Phys. Chem. B. 2011, 115, 7364-7373. (31) Bahmaei, M.; Sabbaghian, E. s.; Farzadkish, E. Development of a method for chlorophyll removal from canola oil using mineral acids. J Am. Oil Chem. Soc. 2005, 82, 679-684. (32) Ghazani, S. M.; Marangoni, A. G. Minor Components in Canola Oil and Effects of Refining on These Constituents: A Review. J Am. Oil Chem. Soc. 2013, 90, 923-932. (33) Sathish, A.; Sims, R. C. Biodiesel from mixed culture algae via a wet lipid extraction procedure. Bioresour. Technol. 2012, 118, 643-647. (34) Chen, L.; Liu, T.; Zhang, W.; Chen, X.; Wang, J. Biodiesel production from algae oil high in free fatty acids by two-step catalytic conversion. Bioresour. Technol. 2012, 111, 208-214. (35) Diosady, L. L. Chlorophyll Removal From Edible Oils. Int. J. Appl. Sci. Eng. 2005, 3. (36) Li, T.; Xu, J.; Wu, H.; GuanghuaWang; Dai, S.; Fan, J.; He, H.; Xiang, W. A Saponification Method for Chlorophyll Removal from Microalgae Biomass as Oil Feedstock. Mar. Drugs 2016, 14. (37) Kleinegris, D. M. M.; Es, M. A. v.; Janssen, M.; Brandenburg, W. A.; Wijffels, R. H. Carotenoid fluorescence in Dunaliella salina. J. Appl. Phycol. 2010, 22, 645-649. (38) Gillbro, T.; Cogdell, R. J. Carotenoid fluorescence. Chem. Phys. Lett. 1989, 158, 312-316. (39) Gilmer, C. M.; Zvokel, C.; Vick, A.; Bowden, N. B. Separation of saturated fatty acids and fatty acid methyl esters with epoxy nanofiltration membranes. RSC Adv. 2017, 7, 55626-55632. (40) Gupta, A.; Bowden*, N. B. Separation of cis-Fatty Acids from Saturated and trans-Fatty Acids by Nanoporous Polydicyclopentadiene Membranes. ACS Appl. Mater. Interfaces 2013, 5, 924-933. (41) Li, X.; Zhang, X.; Yang, Q.; Bao, Z.; Ren, Q.; Zhang, Z.; Xing, H.; Yang, Y. Separation of Highly Unsaturated Fatty Acid Methyl Esters from Model Bio-oils with Ionic Liquid-cosolvent as Extractants. RSC Adv. 2016, 6, 60709-60716. (42) Jiang, S. T.; Shao, P.; Pan, L. J.; Zhao, Y. Y. Molecular Distillation for recovering Tocopherol and Fatty Acid Methyl Esters from Rapeseed Oil Deodoriser Distillate. Biosyst. Eng. 2006, 93, 383-391. (43) Momchilova, S. M.; Nikolova-Damyanova, B. M. Advances in Silver Ion Chromatography for the Analysis of Fatty Acids and Triacylglycerols-2001 to 2011. Anal. Sci. 2012, 28, 837-844. (44) NuriaRubio-Rodríguez; Diego, S. M. d.; Beltran, S.; Jaime, I.; Sanz, M. T.; Rovira, J. Supercritical fluid extraction of fish oil from fish by-products: A comparison with other extraction methods. J. Food Eng. 2012, 109, 238-248. (45) Eun, L. J.; Hyuk, C. Y.; Palanivel, G.; Hae-Soo, K. Concentration of n-3 polyunsaturated fatty acids in cholesterol-reduced cod-liver oil by lipases. Int. J. Food Sci. Tech. 2011, 46, 1462-1468. (46) Simth, A. E. The Crystal Structure of the Urea-Hydrocarbon Complexes. Acta Cryst. 1952, 5, 224. (47) Harris, K. D. M. Fundamental and Applied Aspects of Urea and Thiourea Inclusion Compounds. Supramolecular Chemistry 2007, 19, 47-53. (48) Strocchi, A.; Bonaga, G. Correlation between urea inclusion compounds and conformational structure of unsaturated C18 Fatty acid methyl esters. Chem. Phys. Lipids 1975, 15, 87-94. (49) Ratnayake, W. M. N.; Olsson, B.; Matthews, D.; Ackman, R. G. Preparation of Omega-3 PUFA Concentrates from Fish Oils via Urea Complexation. Eur. J. Lipid Sci. Technol. 1988, 90, 381-386. (50) Nobis, M.; Roberge, D. R. Mastering ozonolysis: production from laboratory to ton scale in continuous flow. Chim. Oggi—Chem. Today 2011, 29, 56-58. (51) Irfan, M.; Glasnov, T. N.; Kappe*, C. O. Continuous Flow Ozonolysis in a Laboratory Scale Reactor. Org. Lett. 2011, 13, 984-987. (52) Katrib, Y.; Martin, S. T.; Hung, H.-M.; Rudich, Y.; Zhang, H.; Slowik, J. G.; Davidovits, P.; Jayne, J. T.; Worsnop, D. R. Products and Mechanisms of Ozone Reactions with Oleic Acid for Aerosol Particles Having Core-Shell Morphologies. J. Phys. Chem. A 2 2004, 108, 6686-6695. (53) C.-W., S.; Y.-P., C.; R.-P., J. Preparation of azelaic acid by oxidation with hydrogen peroxide and ozone. Huaxue Shijie 2007, 48, 612-614. (54) C.-W., S.; Y.-P., C. Preparation of azelaic acid by oxidation with hydrogen peroxide and ozone. Xiandai Huagong 2006, 26, 285-287. (55) C., S.; Y., Y.; W., Y. Study on synthesis and reaction mechanism of azelaic acid by united oxidation. Huagong Keji 2009, 17, 16-19. (56) Z.-C., S.; Y.-G., Z.; W., E.; S.-M., H.; J.-J., G. Production of azelaic acid by the compound solvent-ozonization-catalytic oxidation process from oleic acid. Zhongguo Youzhi 2006, 31, 40-42. (57)-C., S. Z.; Wumanjiang, E.; T. Y., X.; Zhang, Y.-G. Catalysts for oxidation of the ozonolysis product of oleic acid with oxygen to make azelaic acid. Yingyong Huaxue 2006, 23, 161-164.

### Example 3: Description of Work Principle of the Pigment Removing System

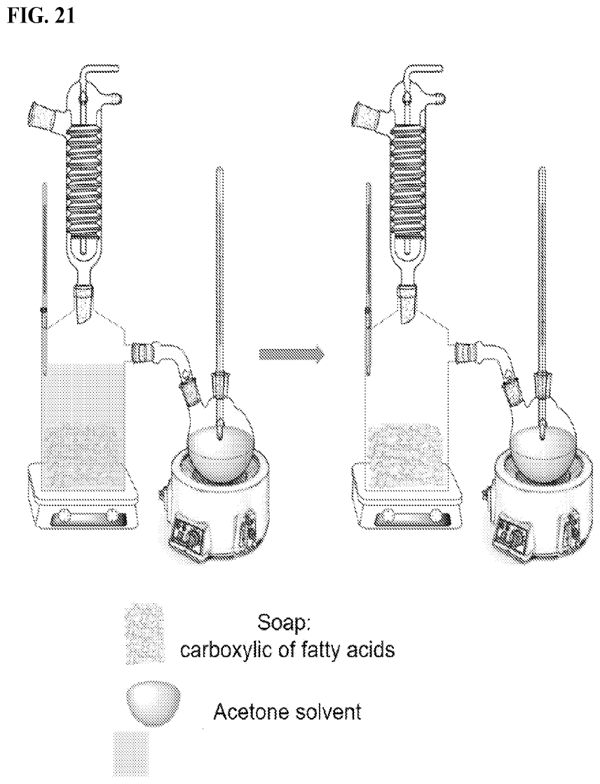

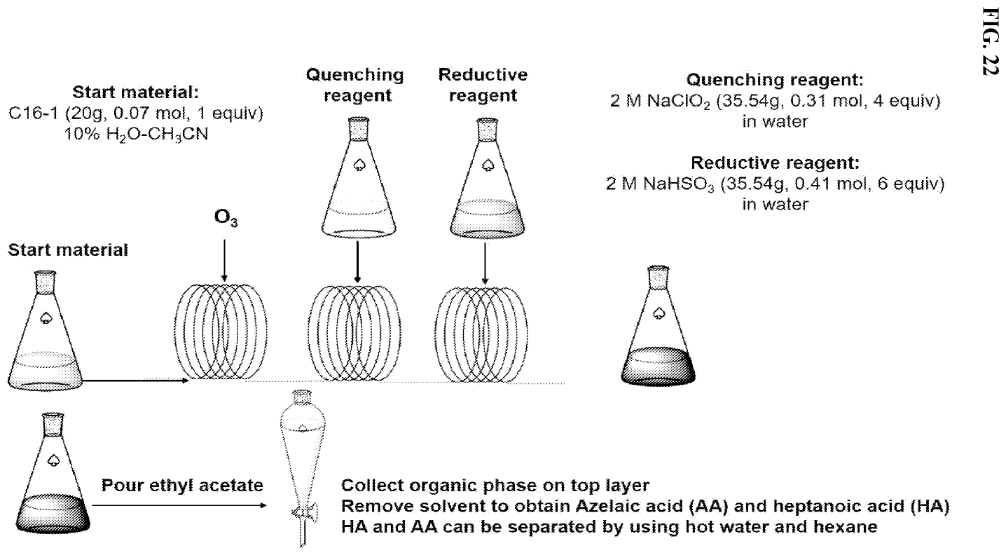

This system can be applied to remove pigments from salt soap with a capacity of 500 g. As shown in FIG. 21, the 2 L glass reagent bottle was charged with 500 g soap and 700-750 mL acetone. Acetone (300-400 mL) was also added to the 1 L two-round bottom flask serving as the pigments trap and the flask was heated to reflux at 70° C. The 2 L glass reagent bottle may be heated to 30-35° C. to increase the rate of extraction of pigment from salt soap. Upon heating, the extraction begins, the condenser solvent from the glass reagent bottle cause the acetone contained pigments to overflow to round bottom flask. Depending on pigments in the fatty acids, the color of extracted solvent may be orange or pink. The extraction process is complete when the organic phase top layer in the glass reagent bottle turns to clear or in light color.

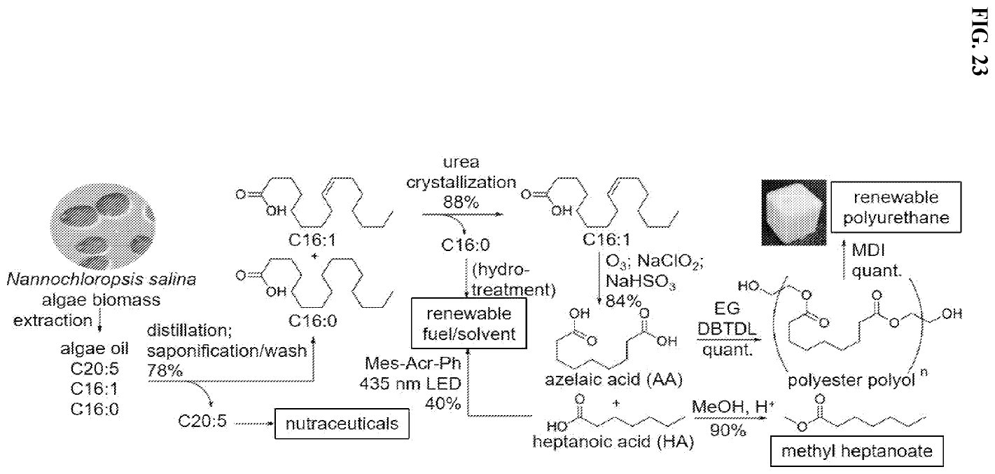

Ozonolysis in batch chemistry is limited at large scale production due to its exothermic reaction, toxicity and thermostability. Therefore, for a large scale production, continuous flow ozonolysis is a preferable method. The schematic representation of the continuous flow ozonolysis is described in FIG. 22.

The obtained Azelaic acid from this invention will be applied to prepare polyester polyol, a precursor for polyurethane synthesis. Azelaic acid is also a precursor in diverse industrial products polymers and plasticizers, as well as a being a component in a number of products related to hair and skin conditioners. Heptanoic acid, a product along with azelaic acid, can be applied in fragrances and artificial flavors, preparation of drugs, and nutritional supplements.

### Example 4: Exemplary Foam Formulation

The azelaic acid and other monomer feedstocks obtained from algae biomass are suitable for the formation of foams, including soft foams such as polyurethanes. In one example, a foam is created as set forth in the Table 5.

TABLE 5
Exemplary foam composition

| General Formulation | Parts per hundred | Grams |
|---|---|---|
| Part A | | |
| Ethylene Azelate polyol Mw = 1000 | 100 | 14.842 |
| Momentive L1507 | 1.74 | 0.258 |
| Bis(dimethylaminoethyl)ether in | 0.1000 | 0.015 |
| dipropylene glycol | | |
| Triethylenediamine in dipropylene | 0.2000 | 0.030 |
| glycol | | |
| Dioctyltin mercaptide | 0.0380 | 0.006 |
| water | 1 | 0.148 |
| Part B | | |
| Polyester pMDI, 19% NCO | Index = 100 | |

The use of algae biomass for obtaining monomer feedstocks and the creation of polymers such as bio-based polyols and polyurethanes therefrom includes the use of polyhydric alcohol (diol, triols, etc.) and dicarboxylic acids. An exemplary but non-exhaustive list of diacids and diols derivable from algae are set forth in the Table 6 below. The combination of any polyhydric alcohol or alcohols with any dicarboxylic acid or acids listed below, when reacted appropriately, produces an algal bio-based polymer, which may be used by itself or in the production of polyurethanes. Each polyhydric alcohol or dicarboxylic acid is itself also suitable for use as an additive in polymers such as polyurethanes.

TABLE 6
Exemplary algae-derived monomers for synthesis
of bio-based polyols and polyurethanes

| # of | | |
|---|---|---|
| Carbons | Polyhydric Alcohol | Dicarboxylic Acid |
| 2 | Ethylene glycol | Oxalic acid |
| 3 | 1,2 Propanediol, 1,3-propanediol, | Malonic acid |
| | glycerol | |
| 4 | 1,3-Butanediol, 1,4-butanediol, | Succinic acid |
| | 2-methyl-1,3-propanediol, | |
| | 2,3-butanediol, trimethylolpropane | |
| 5 | 1,5-pentanediol, neopentyl glycol | Glutaric acid |
| 6 | 1,6-hexanediol, | Adipic acid, 2,5- |
| | 3-methyl-1,5-pentanediol | furandicarboxyic acid |
| 7 | 1,7-heptanediol | Pimelic acid, |
| | | 3,3-dimethyl-1,2- |
| | | cyclopropanedicarboxylic |
| | | acid |
| 8 | 1,8-octanediol | Suberic acid |
| 9 | 1,9-nonanediol | Azelaic acid |
| 10 | 1,10-decanediol | Sebacic acid |

### Example 5: Flexible Polyurethanes, Renewable Fuels, and Flavorings from a Microalgae Oil Waste Stream

Renewable polymers have become an important focus in next-generation materials, and algae biomass offers an environmentally low-impact feedstock that can serve multiple uses. This study aims to develop a scalable methodology for production of microalgae-based polyols for polyurethane synthesis from waste oils derived from algae biomass. Following separation of omega-3 fatty acids from algae oil, residual oils can offer valuable building blocks for petrochemical replacements. However, unlike vegetable oils, algae oils contain organic contaminants, including photosynthetic pigments and hydrophobic cofactors that can complicate preparative methodologies. Here, for example, we convert and purify waste streams from omega-3 depleted Nannochloropsis salina algae oil, with major components consisting of palmitic and palmitoleic acid, into azelaic acid (AA) as a building block for flexible polyurethanes, with a simultaneous production of heptanoic acid that was subsequently converted to flavor and fragrance. Conversion of free fatty acid mixtures into a soft soap allows extraction of organic contaminants, and urea complexation provides isolated palmitoleic acid, which was subsequently ozonolyzed to produce AA and heptanoic acid. Bio-based polyester diols were prepared from AA via esterification to a provide polyol as a monomer for flexible polyurethane foam applications. The heptanoic acid co-product was used to produce the flavoring agent methyl heptanoate and decarboxylated to produce hexane as a renewable solvent. This scalable process can be performed on oils from multiple algal species, offering valuable monomers from a highly sustainable source.

To avoid fossil carbon use, renewable resources that offer the next generation of transportation fuels and chemical feedstocks have been studied in recent years^{1,2}. Although plant oils are known for low toxicity, renewability, and biodegradable properties^{3,4}, use of food crops poses other problems, including high arable land, water, and nutrient use. They also present a competition between biofuel feedstocks and food production⁵. Algae biomass has experienced growing interest as a future source for producing sustainable fuels and materials due to fast growth rate, flexible habitat preferences, and substantial yield⁶. Algae produce more unsaturated fatty acids compared to conventional vegetable oils, which have useful applications to high-value products⁷. In addition, residual microalgae biomass, such as proteins, can be extracted and transformed to high-value biobased products, such as polymers⁸. Algae can be rapidly cultivated on large scales and need not consume arable land or fresh water⁹. One of the major challenges of algae production has been to identify strains with the highest growth rate, lipid content, and lipid productivity for scalable production¹⁰. Identifying the highest and best use of algae oils will be important to ensure sustainability of the market, and omega-3 fatty acids from microalgae currently offer a high-value product within a growing nutraceutical market¹¹. Nannochloropsis strains of green algae offer high titers of eicosapentaenoic acid (EPA, C20:5), roughly 25% of total lipid content, and we reasoned that other high value applications could be explored with the 75% of residual fatty acids. As an application, we identified polyurethane (PU) foam to offer a promising market that is predicted to show continuous growth in coming years, from a USD 54.2 billion in 2018 to over USD 79.8 billion predicted by 2023¹². Without fundamental change, this rapid upward trend of global plastic demand is predicted to offset or reverse any decrease in petroleum demand from the use of renewable energies by 2050¹³. Additional co-products from this process, which include renewable fuels and fragrances, have significant market value¹⁴.

We previously established and developed a large-scale production system for cultivation of a variety of microalgae strains to produce biomass in both photobioractors and open ponds¹⁵, and the resulting algae oils have recently been applied to both biofuel production and for polymeric material synthesis¹. The most abundant components in algae oil are triacylglycerides (TAGs) which can be easily hydrolyzed into saturated and unsaturated free fatty acids, including palmitic acid (C16:0), palmitoleic acid (C16:1), oleic acid (C18:1), and linoleic acid (C18:2). Unsaturations within these fatty acids can be converted into oxygenated functional groups (carboxyl or hydroxyl)¹⁶, which can be valuable for a variety of chemical uses. The oxidative cleavage of olefins of unsaturated fatty acids is industrially carried out by ozonolysis¹⁷ for production of mono- and dicarboxylic acids, which play an important role in a variety of chemical applications including polyesters, polyamides, plasticizers, and pharmaceuticals^{18,19}. The ozonolysis of oleic acid has been shown to be a rapid source of AA and nonanoic acid^{18,19}, both of which have applications in polymer manufacturing.

As monomers with multiple hydroxyl group within their structures, polyols serve as precursors for production of polyurethane products, such as flexible and rigid PU foams²⁰. Recently, bio-based polyols have emerged as renewable and sustainable monomers for the production of PU products. Previous publications showed the syntheses of several bio-polyols from palm, soya, corn, and castor oils^{21,22}, however vegetable oils offer comparatively pure source of TAGs. Algae biomass, by contrast, may be extracted to provide “green crude,” which contains significant photosynthetic pigments and other small organic molecules. In order to prepare homogenously pure feedstock for polymer synthesis, we have developed new methodologies for isolation and conversion of algae fatty acids into PU monomers.

Here, for example, we demonstrate a scalable approach to synthesize flexible PUs from omega-3 depleted algae biomass through the preparation of polyester polyols (FIG. 23). The procedure requires five stages: purification of fatty acids from omega-3 depleted algae oil; isolation of palmitoleic acid (C16:1) from free fatty acids; synthesis of azelaic acid (AA, C9-dicarboxylic acid) from C16:1; polycondensation of ethylene glycol and AA for polyester polyol synthesis; and polymerization with methylenediphenyl diisocyanate (MDI).

We chose the N. salina as a strain for growing algae in large scale because of its established high production of EPA and our ability to grow this strain for high biomass content^{23,15}. Following separation of the low boiling point fractions from the omega-3 fatty acids by distillation, the composition of the resulting mixture was identified by GC-MS by converting free fatty acids into fatty acid methyl esters (FAMEs). These peaks were identified using retention time and mass spectral matching from the NIST reference library. The composition of the fatty acids is listed in FIG. 4 and Table 8: 8.3% myristic acid (C14:0), 28.9% palmitic acid (C16:0), 58.7% palmitoleic acid (C16:1), 2.3% oleic acid (C18:1), and 1.8% linoleic acid (C18:2). The major constituents are palmitoleic acid (C16:1) and palmitic acid (C16:0). Importantly, the high percentage of palmitoleic acid is advantageous as a rich source of monounsaturated fatty acids. Due to the presence of palmitic acid, with melting point of 62.9° C., the raw oil is a solid at room temperature that quickly melts to liquid when it reaches temperature of 70° C. (FIG. 5A).

In addition to these fatty acids, multiple contaminants, including chlorophyll fragments and carotenoids, were identified in the sample, as evidenced by ¹H-NMR (FIG. 6)^{1,24-26}. Unlike seed plants, which store primarily triacylglyceride small molecules, organic extracts from microalgae contain a variety of metabolic components that are insoluble in water but freely soluble in organic solvents such as acetone, diethyl ether, tetrahydrofuran and chloroform^{27,28}. The presence of these pigments with conjugated pi systems may decrease downstream reaction efficiency, therefore their removal is a key process in the production renewable chemicals from algae oil. Multiple studies have described elimination of chlorophylls and carotenoids, which include physical absorption, oxidative treatment, phosphoric acid degumming, precipitation, and bleaching, but are not practical for large scale applications²⁹⁻³³. Recently, Li et al., published a two-step process that includes bleaching combined with saponification to remove chlorophylls from oil, however a large proportion of oil was also lost³⁴.

In this study, we found a practical pathway to not only improve the purity of the algae oil but also to obtain high purified yield with a simple, cost-effective step through saponification. After forming the carboxylate salts of free fatty acids, the obtained soap was washed several times with acetone until the observed filtrate turned from an orange to colorless solution with no UV absorption, indicating that a removal of the pigments was complete. After elimination of pigments, the resulting fatty acids were collected by acidification with aqueous hydrochloric acid. Two classes of soaps could be prepared depending on the cation used. Potassium hydroxide forms a soft soap, retaining a liquid form that is more amenable to large scale acetone extraction. Sodium hydroxide forms a hard soap that requires more solvent and time to remove pigments due to the solid, waxy state.

Treatment of washed soft soap with aqueous hydrochloric acid recovered a mixture of fatty acids with a 85% yield. As shown in FIG. 7, the ¹H-NMR of purified oil indicated that pigment impurities were eliminated. These pigments can be also accurately detected by fluorescence measurement down to parts per million^{35,36}. As shown in FIG. 8, the raw oil displayed a strong emission peak at 668 nm, indicative of π-π* transition^{35,36}, while the purified oil exhibited no emission peak. These color differences between raw oil (FIG. 5A) and purified oil (FIG. 5B) could also be clearly visualized by eye.

In the next step, we employed a practical method to isolate palmitoleic acid (C16:1) from the purified oil mixture. A variety of reported methods for separation of saturated and unsaturated fatty acids have been published, such as urea inclusion complexation^{37,38}, nanoporous membranes^{39,40} ion-liquid solvent extraction⁴¹, molecular distillation⁴², chromatography⁴³, supercritical fluid extraction⁴⁴, and lipase concentration⁴⁵. In many of the above-mentioned methods, urea complexation has proven to be a favorable technique for large-scale isolation of mono-unsaturated fatty acids due to its high separation capacity and simple process. Urea and thiourea are well known to form crystalline complexes with hydrocarbons, saturated fatty acids and other straight-chain molecules⁴⁶. This is made possible by a crystalline tube structure formed by urea that creates an inclusion site for linear compounds when packed densely with the guest molecule⁴⁷. Therefore, the straight-chained palmitic acid and other saturated fatty acids within mixtures crystallize with urea, while the presence of cis-double bonds in unsaturated fatty acids results in a kinked molecular structure^{48,49}, and consequently they cannot enter the hexagonal crystal channel and remain in the solvent. In addition, the urea complexation procedure shown in FIG. 3 benefits from facile recycling of solvents (methanol and hexane) and urea using only physical methods (evaporation and crystallization)^{37,38,49}. Table 8 summarizes the compositions of the initial fatty acid mixture, as well as those of the palmitic and palmitoleic acid after separation by urea complexation.

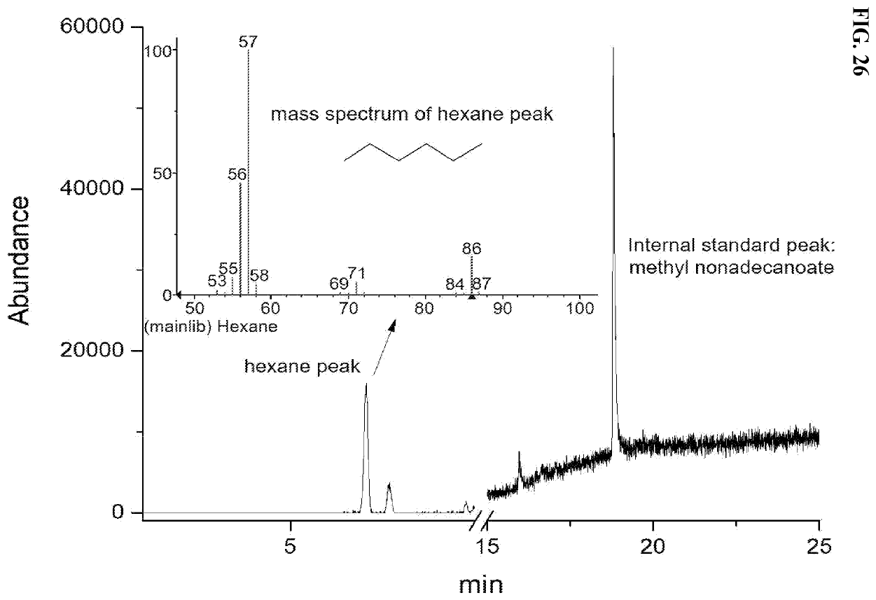

As can be seen from Table 8, palmitoleic acid content of 86%, with a yield of 80%, was obtained from the liquid phase of urea complexation, while the palmitic acid content of 85.9% was recovered from the solid phase. Importantly, most of saturated palmitic acid was removed from the unsaturated fatty acid fraction (FIGS. 9A-9B and FIGS. 25A-25B). The palmitic acid is a white solid that melts at 62.9° C., while palmitoleic acid is yellow liquid at room temperature with a melting point of −0.1° C. FIGS. 10A-10B show the ¹H-NMR spectra of the isolated palmitoleic acid C16:1 and palmitic acid C16-0, respectively.

We next produced AA through oxidative cleavage of palmitoleic acid with ozonolysis. Ozonolysis is well known to offer excellent selectivity through a simple procedure that avoids toxic waste products from oxidants such as nitric acid, permanganate, or dichromate¹⁷. The three-step mechanism for ozonolysis of mono-unsaturated fatty acid has been reported^{17,18}. A green, sustainable oxidizing agent, ozone is preferred as a safe alternative to most other oxidants and catalysts^{17,50}. It is important to note that a continuous flow process with ozone have been developed for industrial applications, enabling conversions on the ton scale of product per day⁵⁰. Despite the advantages of ozonolysis, the process can suffer from sub-optimal yields, reported at around 70% for oleic acid^{17,19}. Published procedures report AAs yield of only 20%, although the ozonolysis of oleic acid has been optimized by high temperatures of up to 150° C. for 2 h¹⁹. Most published ozonolysis procedures quench the intermediate ozonide using 30-60% aqueous H₂O₂ 19, ⁵¹ initially producing aldehyde and carboxylic acid as products⁵¹, and can result in low yields of mono and di carboxylic acid products^{19,52}.

To optimize the quench step, we evaluated a variety of methods, with the goal of complete conversion to carboxylic acid. The combination ozonolysis with oxidation using phosphotungstic acid or tungstic acid and quaternary ammonium salts were found to produce AA at around 70%⁵³⁻⁵⁵. The obtained yield of AA was identical when ozonolysis was combined with metal oxidation catalysts such as Mo-, V-, Mn-, Co-, Fe-, and Pb-oxides and tungstic acid^{56,57}. Ackman, et al., used in situ formed performic acid from H₂O₂ and formic acid incorporation in methanol with oleic acid. Although this approach increased the yield of AA to 95%⁵⁸, it is only suitable for small laboratory scale due to safety concerns. Here, instead of quenching with oxidative cleavage once completing ozonolysis, solvent was first removed under vacuum, followed by addition of H₂O₂ and HCO₂H⁵⁸. As the formed ozonides or peroxides are potentially explosive, further workup raised safety concerns.

Recently, a mild one-pot ozonolysis oxidation process of olefins was reported to synthesize carboxylic acids with yields of up to 98%⁵⁹. This process, which employs sodium chlorite as an oxidant, is reported to be a scalable procedure, safely converting over 20 kg of an alkene starting material in a high yield and purity⁵⁹. Therefore, we chose ozonolysis in combination with oxidative cleavage using sodium chlorite to prepare AA from palmitoleic acid C16:1. Here we report a mild one-pot, metal-free process to oxidatively cleave palmitoleic acid to AA and heptanoic acid⁵⁹. Ozonolysis was conducted in an aqueous organic solvent (10% H₂O-MeCN), and the generated intermediates were converted to desired carboxylic acids by oxidation with sodium chlorite, which was then followed by a reductive quench with sodium bisulfite. AA was extracted away from heptanoic acid byproduct and hexane solvent with hot water, followed by recrystallization. This ozonolysis-oxidation procedure obtained AA and heptanoic acid in yields of 83%. For mass production of HA and AA on kg scale, a continuous-flow ozonolysis process can be implemented to improve safety through high transfer rates and small reaction volumes^{50,51}.

A linear polyester with an average molecular weight of 4000 and a weight average molecular weight of 10,600 was prepared through acid-catalyzed polycondensation of AA with ethylene glycol. A detailed procedure is described in supporting information. The acid number of the polyester polyol was in the range of 1-3, indicating near-completeness of the polymerization reaction. The structure of polyester polyol was characterized by ¹H-NMR spectroscopy (FIG. 14) and FT-IR (FIG. 15). The IR spectrum displayed characteristic polyester polyol peaks at 3500 cm⁻¹ and 1730 cm⁻¹, showing OH and C═O stretching from the hydroxyl and ester carbonyl groups, respectively. The double peaks at 2927 and 2852 cm⁻¹ were consistent with C—H stretching from hydrocarbons, and the large peak at 1160 cm⁻¹ in the fingerprint region was also identified as ester C—O stretching. The broader OH peak in the algae-based polyol maybe because of a combination of lower hydroxyl number and/or residual acid content. H¹-NMR indicated the existence of both terminal alcohols and ester groups in the obtained polyol sample. The molecular weight, OH number and acid number are summarized in Table 9.

As illustrated in FIG. 16, the obtained polyol shows a melting point of 25° C. and glass transition temperature (Tg) within the range of −25 to −16.5° C. A broad and weak endothermic above Tg is due to the crystallization of a short chain in the polyol structure. The polyol exhibits a Tg below room temperature, a characteristic property which indicates its elastomer behavior. A high mobility of short chains in the polyol results in the elasticity at temperature above the Tg⁶⁰.

The flexible polyurethanes synthesized here can be described as a low density water-blown foam, similar to those commonly made in the molded and slabstock industries. The foam formulation (Table 10) was chosen to represent a basic flexible polyurethane that can be used in a variety of applications ranging from furniture to automotive cushioning. Resultant polyurethane cubes were fabricated using a stainless steel mold, as described in the supporting information. This allows for standard compression and hardness tests to be done with accuracy and reproducibility. We used a compression-decompression cycle (ASTM D3574-C) to determine how quickly the foam cubes can respond to stress, as well as their load-bearing capacity. This allowed us to understand whether the foam is springy, and returns quickly to its original shape, or exhibits viscoelastic behavior, i.e., ‘memory effect’. By measuring the force relative to displacement, integrating the compression curve, subtracting the same integral for decompression, we determined energy loss. We found that our synthesized polyurethanes gave 51% energy loss, indicating that that they deform easily and cannot quickly revert to their native state, and exhibited a pronounced memory effect. The nine-carbon AA chain could be responsible for this; relative to polyols based on shorter diacids, AA-based polyols have more bonds capable of free rotation and fewer rigid ester bonds. It is important to note that high energy loss is not an indicator of poor performance. In fact, in certain products such as mattresses, this effect is desirable. The DSC curve of resulting algae-derived PU foam is presented in FIG. 17. The curve shows a glass transition temperature of 26.5° C. in a heating scan of PU foam. PU foam is a 3D network structure of mobile soft segments (polyol) and rigid hard segments (MDI). The PU matrix itself is highly cross-linked⁶¹. Therefore, the flexibility of polyol chains lead to good elasticity of PU foam at room temperature.

The properties of resulting algae-based PU cubes are shown in Table 7. Mechanical properties strongly depend on the degree of crosslinking and network structure of the PU foam. The diisocyanate react with algae polyol leads to urethane linkage which generates the hard domain of PU foam because of the possibility of association by hydrogen bond while the high molecular weight and mobility of algae polyol represent the soft domain, resulting in superior mechanical properties. The hysteresis and peak force values trend well with shore hardness, and the more rigid algae PU cubes demonstrate lower energy loss and higher peak force as is expected.

TABLE 7
Azelaic Polyol Foam Cube Properties

| | Avg. | Avg. | Avg. | Avg. Peak |
|---|---|---|---|---|
| | Density | Hardness | Hysteresis | Force |
| Formula | (kg/m³) | (Shore A) | (%) | (N) |
| Photosynthetic PU foam | 297 ± 4 | 30 ± 3 | 51 ± 3 | 217 ± 17 |

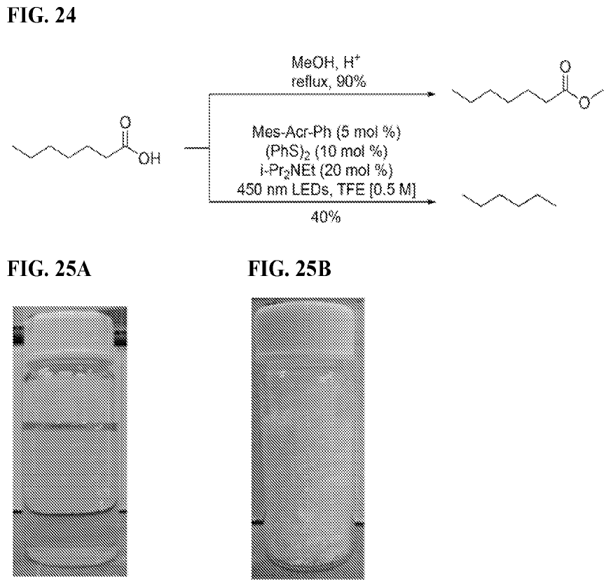

The other ozonolysis co-product was heptanoic acid. Methyl heptanoate, an ester from the condensation of heptanoic acid and methanol, is widely used in the flavor and fragrance industry, with a fruity, green aroma and flavor⁶²⁻⁶⁴. We prepared methyl heptanoate from our algae based heptanoic acid via esterification with a 90% yield (FIG. 24 and Scheme 6). Methyl heptanoate can be applied in a broad range of consumer products, including food, beverages, fragrances, cosmetics, personal care and household products.⁶³

In addition, carboxylic acids can be converted to hydrocarbons and other chemicals by decarbonylation, decarboxylation, and deoxygenation⁶⁵⁻⁶⁸. These conversion can be challenging due to the requirement for higher temperatures (300-400° C.)⁶⁶, nobel metal catalysts (Pd, Pt), low reaction rates⁶⁵ and rapid catalyst deactivation⁶⁹. In recent years, photoredox catalysis have emerged at the forefront of synthetic organic chemistry for decarboxylative functionalizations⁷⁰, including decarboxylative alkylation⁷¹, decarboxylative vinylation⁷² and decarboxylative arylation⁷³. Photoredox catalysis utilizes the energy of light to accelerate a chemical reaction via single electron transfer⁷⁴, which avoids the use of traditional chemical reagents that are often toxic or hazardous. A direct organocatalytic protocol for the decarboxylation of carboxylic acids to alkanes has been reported⁷⁰. We applied this procedure with algae-based heptanoic acid (FIG. 24, Scheme 6, and FIG. 26). The procedure produced renewable hexane at 40% yield by treatment with 450 nm light and a photocatalyst. Hexane has innumerable uses, including edible oil extraction, degreasing agents, and as fuel additives.

To explore the economic value of this process, we can provide a conservative estimate based on the market value of AA and methyl heptanoate. Assuming theoretical (quantitative) yields, palmitoleic acid (16:1) represents ˜50% of crude fatty acid waste stream mass, and the ozonolysis process provides 70% AA (by molecular weight). Taking into account our 58% overall yield to prepare AA (over 3 steps), and that we purchase the crude algae fatty acid waste stream for $1/kg (˜$0.25/mol), our cost to produce AA is $1.22/mol ($6.48/kg). The bulk price of AA is currently $8.85/mol ($47/kg), which is 7.25-fold value increase. Using similar metrics, our cost to produce methyl heptanoate is $2.02/mol ($14.00/g), and bulk price is currently $72.10/mol ($500/kg). This is a 36-fold value increase, although there is a limited market for these flavors/fragrances.

In summary, AA and HA were successfully prepared from an algae oil waste stream and converted into a flexible PU foam, a bio-based flavoring, and renewable solvent (Scheme 1). After separating EPA for nutraceutical uses from algae oil, the resulting waste stream consists of a mixture of fatty acids is purified and separated into C16-1 and C16-0 in a yield of 85-88% by urea complexation. Subsequent ozonolysis cleavage results in AA and HA, both in 84% yield. This study indicates that AA derived from an algae-sourced waste stream has the potential to support material production of polyester polyols, a precursor for polyurethane synthesis, and renewable solvents. Valorizing a waste stream from omega-3 fatty acid production in this manner, which would otherwise be converted into liquid fuels, provides added cost benefits for algae biomass production. Heptanoic acid co-product can be converted into methyl heptanoate (90% yield), a valuable product for the flavors and fragrances industry, and renewable hexane (40% yield). We plan to optimize and scale these procedures to enable large scale production of AA and HA, where continuous flow ozonolysis will allow achievement of large-scale capacities. The exploration and utilization of algae biomass to prepare high value products offers tools to sustainably transition from petrochemicals to renewable chemical feedstocks.

### REFERENCES FOR EXAMPLE 5

(1) P. Roesle, F. Stempfle, S. K. Hess, J. Zimmerer, C. R. Bártulos, B. L. A. Eckert, P. G. Kroth and S. Mecking, Angew Chem Int Ed 53:6800-6804 (2014). (2) M. A. R. Meier, J. O. Metzger and U. S. Schubert, Chem Soc Rev 36:1788-1802 (2007). (3) D. Singh, R. Bhoi, A. Ganesh and S. Mahajani, Energy Fuels 28:2743-2753 (2014). (4) A. B. Chaudhari, P. D. Tatiya, R. K. Hedaoo, R. D. Kulkarni and V. V. Gite, Ind Eng Chem Res 52:10189-10197 (2013). (5) R. Herrmann, C. Jumbe, M. Bruentrup and EvansOsabuohien, Biomass Bioenerg 114:100-111 (2018). (6) J. L. Blatti and M. D. Burkart, J Chem Educ 89:239-242 (2012). (7) Waghmare, S. Patil, J. G. LeBlanc, S. Sonawane and S. S. Arya, Algal Res 31:99-106 (2018). (8) S. Kumar, E. Hablot, J. L. G. Moscoso, W. Obeid, P. G. Hatcher, B. M. DuQuette, D. Graiver, R. Narayan and V. Balan, J Mater Sci 49:7824-7833 (2014). (9) R. Slade and A. Bauen, Biomass Bioenerg 53:29-38 (2013). (10) M. Sara, S. K. Brar and J. F. Blais, RSC Adv 6:90547-90558 (2016). (11) M. A. Borowitzka, J Appl Phycol 25:743-756 (2013). (12) A. Austin and D. A. Hicks, PU Magazine 14:1-15 (2017). (13) IEA, IEA summary report https://webstore.iea.org/the-future-of-petrochemicals:(2018). (14) https://wwwresearchandmarketscom/(2019). (15) N. G. Schoepp, R. L. Stewart, V. Sun, A. J. Quigley, D. Mendola, S. P. Mayfield and M. D. Burkart, Bioresour Technol 166:273-281 (2014). (16) A. E. Kerenkan, F. Béland and T.-O. Do, Catal Sci Technol 6:971-988 (2016). (17) A. Kockritz and A. Martin, Eur J Lipid Sci Technol 113:83-91 (2011). (18) V. Benessere, M. E. Cucciolito, A. D. Santis, M. D. Serio, R. Esposito, F. Ruffo and R. Turco, J Am Oil Chem Soc 92:1701-1707 (2015). (19) A. A. H. Kadhum, B. A. Wasmi, A. B. Mohamad, A. A. Al-Amiery and M. S. Takriff, Res Chem Intermed 38:659-668 (2012). (20) O. Gómez-Jiménez-Aberasturi and J. R. Ochoa-Gómez, J Chem Technol Biotechnol 92:705-711 (2017). (21) R. Gu, S. Konar and M. Sain, J Am Oil Chem Soc 89:2103-2111 (2012). (22) Z. S. Petrovic, X. Wan, 0. Bilic, A. Zlatanic, J. Hong, I. Javni, M. Ionescu, J. Milic and D. Degruson, J Am Oil Chem Soc 90:1073-1078 (2013). (23) A. Sukenik, Y. Carmeli and T. Berner, J Phycol 25:686-692 (1989). (24) M. Najdanova, H. U. Siehl and S. Berger, J Phys Org Chem 31:e3802 (2018). (25) R. D. Willows, Y. Li, H. Scheer and M. Chen, Org Lett 15:1588-1590 (2013). (26) A. M. LaFountain, C. Pacheco, R. O. Prum and H. A. Frank, Arch Biochem Biophys 539:133-141 (2013). (27) N. E. Craft and J. H. Soares, J Agric Food Chem 40:431-434 (1992). (28) A. P. Gerola, T. M. Tsubone, A. Santana, H. P. M. d. Oliveira, N. Hioka and W. Caetano, J Phys Chem B 115:7364-7373 (2011). (29) M. Bahmaei, E. s. Sabbaghian and E. Farzadkish, J Am Oil Chem Soc 82:679-684 (2005). (30) S. M. Ghazani and A. G. Marangoni, J Am Oil Chem Soc 90:923-932 (2013). (31) A. Sathish and R. C. Sims, Bioresour Technol 118:643-647 (2012). (32) L. Chen, T. Liu, W. Zhang, X. Chen and J. Wang, Bioresour Technol 111:208-214 (2012). (33) L. L. Diosady, Int J Appl Sci Eng 3:(2005). (34) T. Li, J. Xu, H. Wu, GuanghuaWang, S. Dai, J. Fan, H. He and W. Xiang, Mar Drugs 14:(2016). (35) D. M. M. Kleinegris, M. A. v. Es, M. Janssen, W. A. Brandenburg and R. H. Wijffels, J Appl Phycol 22:645-649 (2010). (36) T. Gillbro and R. J. Cogdell, Chem Phys Lett 158:312-316 (1989). (37) D. G. Hayes, Y. C. Bengtsson, J. M. V. Alstine and F. Setterwall, J Am Oil Chem Soc 75:1403-1409 (1998). (38) Bin Jiang, Yan Liu, L. Zhang, Yongli Sun, Y. Liu and X. Liu, J Chem Soc Pak 36:1013-1020 (2014). (39) C. M. Gilmer, C. Zvokel, A. Vick and N. B. Bowden, RSC Adv 7:55626-55632 (2017). (40) A. Gupta and N. B. Bowden, ACS Appl Mater Interfaces 5:924-933 (2013). (41) X. Li, X. Zhang, Q. Yang, Z. Bao, Q. Ren, Z. Zhang, H. Xing and Y. Yang, RSC Adv 6:60709-60716 (2016). (42) S. T. Jiang, P. Shao, L. J. Pan and Y. Y. Zhao, Biosyst Eng 93:383-391 (2006). (43) S. M. Momchilova and B. M. Nikolova-Damyanova, Anal Sci 28:837-844 (2012). (44) NuriaRubio-Rodríguez, S. M. d. Diego, S. Beltran, I. Jaime, M. T. Sanz and J. Rovira, J Food Eng 109:238-248 (2012). (45) L. J. Eun, C. Y. Hyuk, G. Palanivel and K. Hae-Soo, Int J Food Sci Tech 46:1462-1468 (2011). (46) A. E. Simth, Acta Cryst 5:224 (1952). (47) K. D. M. Harris, Supramolecular Chemistry 19:47-53 (2007). (48) A. Strocchi and G. Bonaga, Chem Phys Lipids 15:87-94 (1975). (49) W. M. N. Ratnayake, B. Olsson, D. Matthews and R. G. Ackman, Eur J Lipid Sci Technol 90:381-386 (1988). (50) M. Nobis and D. R. Roberge, Chim Oggi—Chem Today 29:56-58 (2011). (51) M. Irfan, T. N. Glasnov and C. O. Kappe*, Org Lett 13:984-987 (2011). (52) Y. Katrib, S. T. Martin, H.-M. Hung, Y. Rudich, H. Zhang, J. G. Slowik, P. Davidovits, J. T. Jayne and D. R. Worsnop, J Phys Chem A 2 108:6686-6695 (2004). (53) S. C.-W., C. Y.-P. and J. R.-P., Huaxue Shijie 48:612-614 (2007). (54) S. C.-W. and C. Y.-P., Xiandai Huagong 26:285-287 (2006). (55) S. C., Y. Y. and Y. W., Huagong Keji 17:16-19 (2009). (56) S. Z.-C., Z. Y.-G., E. W., H. S.-M. and G. J.-J., Zhongguo Youzhi 31:40-42 (2006). (57) S. Z.-C., E. Wumanjiang, X. T. Y. and Y.-G. Zhang, Yingyong Huaxue 23:161-164 (2006). (58) R. G. Ackman, M. E. Retson, L. R. Gallay and F. A. Vandenheuvel, Can J Chem 39:1956-1963 (1961). (59) B. M. Cochran, Synlett 27:245-248 (2016). (60) M. N. Baharu, A. A. H. Kadhum, A. A. Al-Amiery and A. B. Mohamad, Green Chem Lett Rev 8:31-38 (2015). (61) K. N. Raftopoulos, J. Pagacz, J. Ozimek, S. Koutsoumpis, S. Michalowski, E. Hebda, J. Pielichowski and K. Pielichowski, Polym Bull 76:2887-2898 (2019). (62) P. Schieberle, W. Grosch and M. Rychlik, Dt Forschungsanst für Lebensmittelchemie 1-63 (1998). (63) M. C. Qian, J Agric Food Chem 67:13775-13777 (2019). (64) N. Innocente, S. Moret, C. Corradini and L. S. Conte, J Agric Food Chem 48:3321-3323 (2000). (65) J. A. Lopez-Ruiz and R. J. Davis, Green Chem 16:683-694 (2014). (66) Y. Bie, A. Gutierrez, T. R. Viljava, J. M. Kanervo and J. Lehtonen, Ind Eng Chem Res 52:11544-11551 (2013). (67) G. Cahiez, A. Moyeux, O. Gager and M. Poizat, Adv Synth Catal 355:790-796 (2013). (68) M. Z. Hossain, A. K. Jhawar, M. B. I. Chowdhury, W. Z. Xu, W. Wu, D. V. Hiscott and P. A. Charpentier, Energy Fuels 31:4013-4023 (2017). (69) S. Lestari, P. Mäki-Arvela, H. Bernas, O. Simakova, R. Sjöholm, J. Beltramini, G. Q. M. Lu, J. Myllyoja, I. Simakova and D. Y. Murzin, Energy Fuels 23:3842-3845 (2009). (70) J. D. Griffin, M. A. Zeller and D. A. Nicewicz, J Am Chem Soc 137:11340-11348 (2015). (71) C. P. Johnston, R. T. Smith, S. Allmendinger and D. W. C. MacMillan, Nature 536:322-325 (2016). (72) A. Noble, S. J. McCarver and D. W. C. MacMillan, J Am Chem Soc 137:624-627 (2015). (73) Z. Zuo, D. T. Ahneman, L. Chu, J. A. Terrett, A. G. Doyle and D. W. C. MacMillan, Science 345:437-440 (2014). (74) M. H. Shaw, J. Twilton and D. W. C. MacMillan, J Org Chem 81:6898-6926 (2016).

### Example 6: Materials and Methods

Materials. Nannochloropsis salina was obtained from the National Center for Marine Algae and Microbiota, Maine, USA. Chemical reagents were purchased from Fisher Chemical, Macron Fine Chemicals, Sigma Aldrich, Acros, Fluka, Alfa Aesar Chemicals or TCI-America. All chemicals were regent grade and used without further purification. Analytical grade solvents such as acetone, hexane, and methanol were purchased from Fisher Chemical and used as received. Deuterated NMR solvents such as chloroform-d, DMSO-d6 were purchased from Cambridge Isotope Laboratories.

Equipment. Gas chromatography mass spectrometry (GC-MS) was run on an Agilent 7890A GC system connected to a 5975C VL MSD quadrupole MS (EI). Samples were separated on a 60 m DB23 Agilent GCMS column using helium as carrier gas and a gradient of 110° C. to 200° C. at 15° C.min⁻¹, followed by 20 minutes at 200° C. ¹H NMR spectra was recorded on a JOEL ECA 500 or a Varian VX500 spectrometer equipped with an Xsens Cold probe. ATR-FTIR was performed on a Perkin Elmer Spectrum RXI equipped with a ZnSe 1 mm ATR cell. 18 scans were taken at a 1 cm⁻¹ resolution. Gel permeation chromatography (GPC) was performed on Agilent GPC/SEC system, the samples were run in chloroform at 45° C. using a refractive index detector and analyzed against polystyrene standard. Fluorescence spectra were recorded at room temperature on a Thermo Scientific Varioskan LUX at the excitation wavelength of 350 nm. Differential Scanning calorimetry (DSC) was measured on Perkin-Elmer in a temperature range of −50 to 600° C. under Ar at flow rate of 20 mL min⁻¹ with a heating rate of 10 K min⁻¹. All data are referred to the second heating cycle. Ozone is produced from the Triogen LAB2B Ozone generator. Irradiation of photochemical reaction was carried out using 12V 5630 LED Strip Light purchased from IEKOV. The wavelength of Blue LEDs light was measured by the Compact CCD Spectrometer.

Step 1: Purification of Fatty Acids from Biomass.

Nannochloropsis salina¹ was chosen as a strain for outdoor growth of microalgae biomass due to its robust growth and ability to accumulate high concentrations of polyunsaturated fatty acids². The procedure for algae culturing and harvesting of biomass has been described in our previous publication³. The harvested algae paste was collected and dried by centrifugation and storing at −20° C. and the Triacylglycerides (TAGs) were extracted from using hexane and isopropanol using a liquid-liquid extraction technique⁴. After a process of TAG hydrolysis, omega-3 fatty acids were isolated using fractional distillations, providing a mixture of saturated and monounsaturated fatty acids methyl esters (FAMEs). Acidic hydrolysis of these FAMEs provided a raw oil containing a mixture of saturated and monounsaturated fatty acids partially contaminated with photosynthetic pigments other small molecules.

To remove non-fatty acid contaminants, saponification was carried out on 127 g of FAME mixture with 400 mL aqueous KOH 3N. The collected soft soap was washed with acetone several times obtain the purified soap. The fatty acids were recovered by acidification with 6N aqueous HCl (FIG. 2). Yield: 98 g, 78%.

Step 2: Isolation Mono-Unsaturated Fatty Acid C16-1.

Palmitoleic acid was isolated from a mixture of FFAs using a urea complexation method under optimal conditions (FIG. 3)^{6,7}. The free fatty acids (100 g) were mixed with 123 g urea in 770 mL methanol, then heated at 70° C. until the mixture became a homogeneous solution. The resulting mixture was slowly cooled to room temperature for 30 minutes before storing overnight at 4° C. for crystal formation. The crystals were separated from liquid by filtration under vacuum. Methanol was removed from the filtrate with a rotary evaporation, which was then washed with warm water (70° C.) and extracted with an equal volume of hexane. The hexane layer containing mono-unsaturated fatty acid was dried with anhydrous sodium sulfate before solvent removal by rotary evaporation to obtain pure palmitoleic acid (49.8 g, 85% yield). The signal at δ 0.71-1.11 corresponds to the terminal alkyl methyl, while the four peaks between δ 1.15-2.46 are assigned to the methylene groups nearest to the double bond and carboxyl group. FIGS. 10A-10B show the ¹H NMR spectra of the isolated palmitoleic acid C16-1 and palmitic acid C16-0. The palmitoleic acid C16-1 (FIG. 10A) is identified by the high intensity signal at δ 5.24-5.50 belong to double bond. In contrast, there is a very low intensity signal at δ 5.22-5.44 in the ¹H NMR spectrum of palmitic acid C16-0 (FIG. 10B), indicative of the presence of small amount of C16-1. These findings agreed with GC-MS data (FIGS. 9A-9B).

Step 3: Oxidative Cleavage of C16-1.

Azelaic acid was synthesized by oxidative cleavage of mono-unsaturated fatty acid C16-1 with ozone as shown in FIG. 23. The procedure is similar to the process of synthesis of azelaic acid from oleic acid⁸⁻¹¹, except that a quench reaction was accomplished by sodium chlorite (NaClO₂)¹². C16-1 (20 g, 0.07 mol, 1 equiv) was dissolved in a mixture of 150 mL acetonitrile and 15 mL H₂O. The solution was cooled to 0° C. in ice bath and treated with ozone until the reaction complete, as confirmed by TLC. Once the ozonolysis completed, a 157 mL aqueous solution of 2M sodium chlorite (35.54 g, 0.31 mol, 4 equiv) was added dropwise into the cold reaction with the temperature controlled at 0° C. The reaction mixture turns yellow upon sodium chlorite addition. After standing overnight at room temperature, the mixture was reduced by slow addition of aqueous 2M sodium bisulfite (166 mL, 34.6 g, 0.33 mol, 4 equiv) under controlled temperature of 0° C. Once completed, the solution turned colorless and clarified, and the mixture was stirred for 10 minutes. Ethyl acetate (100 mL) added, and the two layers were separated. The organic phase, with azelaic acid and heptanoic acid, was dried by rotary evaporator to obtain a white paste product, which was diluted with hexane and extracted with hot water. Upon cooling the aqueous phase, azelaic acid (AA) formed as white crystals, which were filtered, washed several times with cold water, and dried (12.4 g, 84% yield.) The hexane layer containing heptanoic acid was dried over Na₂SO₄, filtered, and concentrated as an oily liquid (8.5 g, 83% yield). The product was analyzed by GC-MS (FIG. 11) and ¹H & ¹³C NMR (FIG. 12A and FIG. 13A). Due to the relatively high boiling point of azelaic acid (286° C.), analysis was performed by converting azelaic acid into the dimethyl ester and analyzing by GC-MS (FIG. 11). The dimethyl azelate was identified by matching mass spectral data to the NIST library database. As shown in FIG. 11, the retention time of the dimethyl azelate appeared at 13.16 minutes. The mass spectra of this observed peak was characterized with a cluster of fragmentation patterns and ions, which match well with the mass spectra reference library of dimethyl azelate. As shown in FIG. 12A, the signals at δ 1.0-1.8 ppm was assigned to methyl and methylene groups of azelaic acid, a triplet peak at δ 2.2 ppm correspond to C—H_{aliphatic} protons near the carboxylic group, and a broad signal around δ 12 is from carboxylic acid proton. The two peaks between δ 2.4 and 3.3 ppm belong to DMSO and water, respectively. Analyzed data of azelaic acid: ¹H NMR (500 MHz, DMSO-d₆) δ=11.95 (s, 1H), 2.15 (t, J=7.4, 2H), 1.44 (p, J=6.9, 2H), 1.21 (d, J=6.3, 4H). ¹³C NMR (126 MHz, DMSO-d₆) δ=175.10, 34.16, 28.95, 24.97. HR-ESI-MS calcd. for azelaic acid—C₉H₁₆O₄[M-H]⁻: 187.22, found 187.25.

The heptanoic acid product was also produced at over 85%, as analyzed by 1H & NMR (FIG. 12B and FIG. 13B). Analyzed data of heptanoic acid: ¹H NMR (500 MHz, DMSO-d₆) δ=2.13 (t, J=7.4, 1H), 1.50-1.38 (m, 1H), 1.26-1.17 (m, 4H), 0.81 (t, J=7.0, 1H). ¹³C NMR (126 MHz, DMSO-d₆) δ=175.17, 34.18, 31.48, 29.53, 28.72, 24.97, 22.49, 14.38. HR-ESI-MS calcd. for heptanoic acid—C₇H₁₄O₂[M-H]⁻: 129.18, found 129.23.

Scheme 4. Ozonolysis of Palmitoleic Acid.

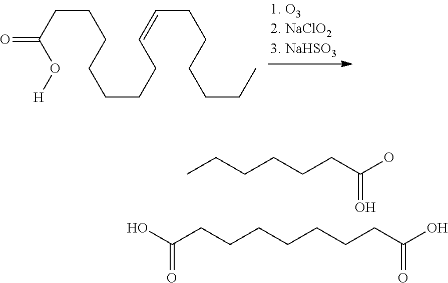

Step 4: Polycondensation of Ethylene Glycol and Azelaic Acid for Polyester Polyol Synthesis.

The reaction procedure for polyol synthesis was adapted from a literature report¹³. To a 100 mL 3-neck flask, 17.8 g of azaleic acid and 8.7 g of ethylene glycol were combined. One neck was fitted with a gas inlet to allow dry nitrogen to flow through at a fixed flow rate to around 80 mL/min. The central neck was fitted with a thermometer for temperature verification. The right neck was attached to a Dean-Stark apparatus to collect the water released by the esterification reaction. The apparatus was heated to 140° C. using a heating mantle with stirring to facilitate melting of the azelaic acid. At this point, 10 μL of dibutyltin dilaurate was added and the temperature gradually increased to 200° C. over the course of an hour. The reaction was allowed to proceed for a further 14 hours, until all of the monomers were consumed.

Scheme 5. Synthesis of Polyester Polyols.

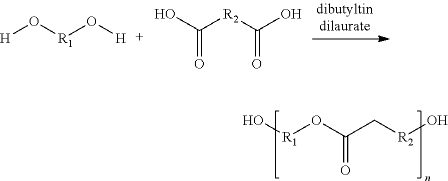

The polyols were characterized by OH number and acid number according to ASTM E1899 and D664, respectively using a Mettler Toledo G20S auto-titrator using a non-aqueous electrode. For the OH number titrations, four replicates of between 0.1 and 0.3 grams of polyol were reacted with p-toluene sulfonyl isocyanate (TSI) to form the carbamate, which was subsequently titrated with a standardized solution of 0.1M tetrabutylammonium hydroxide in acetonitrile. The acid number titrations were performed in duplicate. 1 g of sample was diluted in a 50:49:1 solution of toluene, isopropanol, and water, then titrated with a standardized solution of 0.1M KOH in isopropanol.

Step 5: Polyurethane polymerization with methylenediphenyl diisocyanate (MDI)

A stainless steel mold with three 1″ cube slots was used to fabricate the foam samples. The mold was heated in an oven to 50° C. to ensure that the exothermic urethane reaction is sustained. Mold release (Stoner 5236) was applied by lightly spraying on to the mold sidewalls, to ensure ease of demold. Polyols were heated to 50° C. to liquefy and reduce viscosity. All other components were used at room temperature. Polyol, catalyst, surfactant, water and isocyanate components were weighed into a cup and mixed with a DAC 600.1 Flacktek speed mixer at 2000 rpm for 17 seconds. The cube mold was placed on a balance. Each cube was hand poured from the cup into the mold to ensure consistent mass across cubes. The mold was then sealed and cured in an oven for 1 hour at 50° C., and then cooled to room temperature before demolding the cubes.

This study characterized four physical properties of the polyester polyurethane material: density, hardness, hysteresis, and peak force. The mass of each foam cube was measured on an analytical balance with an accuracy of plus or minus 0.01 grams. Density was determined by dividing the mass by the mold volume for each 1 inch cube. Hardness was measured by pressing a digital shore A durometer, made by FstDgte, into the center of each cube according to ASTM method D2240. The reported hardness is an average of the durometer measurements from all six faces of each cube.

Hysteresis and peak force were calculated using an AFG 2500N compression tester by MecMesin, with a MultiTest-dV sample stage. The test method was a compression of 50% of the original height of each cube, at a speed of 100 mm per minute. This instrument output a curve displaying each data point of force versus height of displacement. Energy loss was calculated as the integral under the curve for the compression, minus the integral under the curve for decompression. Percent hysteresis was calculated dividing the energy loss by the energy in. The peak force was measured on the 10th cycle of compression, in order to illustrate load-bearing capacity of the material.

TABLE 8
Fatty acid components and contents
in samples analyzed by GC-MS

| | Samples | | |
|---|---|---|---|
| | Initial fatty | Isolated palmitoleic | Isolated palmitic |
| | acids | acid | acid |
| | Percentage of | C16-1 | C16-0 |
| Fatty acids | fatty acid | Percentage of fatty | Percentage of fatty |
| contents | methyl ester | acid methyl ester | acid methyl ester |
| C14-0 | 8.3 | 3.4 | 4.9 |
| C16-0 | 28.9 | 8 | 85.9 |
| C16-1 | 58.7 | 86 | 8.8 |
| C18-1 | 2.3 | 1.8 | 0.4 |
| C18-2 | 1.8 | 0.8 | 0 |

Note:
C14-0: “14” is the number of carbon atoms in the fatty acids molecule, while “0” is the number of carbon-carbon double bonds.

TABLE 9
Properties of polyester polyol

| Polyol properties | Photosynthetic azelaic polyol |
|---|---|
| OH number, mg KOH/g | 109 ± 2 |
| Acid number, mg KOH/g | 0.28 ± 0.02 |
| GPC analysis - M_{w}, gmol⁻¹ | 10600 |
| (weight average molecular weight) | |
| GPC analysis - Mₙ, gmol⁻¹ | 4000 |
| (number average molecular | |
| weight) | |
| GPC analysis - poly dispersity | 2.6 |
| index (PDI) | |

TABLE 10
Table for the cube foam PU formulation

| Component | Parts per hundred | Weight (g) |
|---|---|---|
| Photosynthetic polyol | 100 | 14.482 |
| Momentive L1507 | 1.74 | 0.258 |
| Niax A1 | 0.1 | 0.015 |
| TEDA | 0.2 | 0.03 |
| Fomrez UL-29 | 0.03 | 0.006 |
| Water | 1 | 0.148 |
| MDI | 89.9 | 13.02 |

Hydrodecarboxylation of heptanoic acid via Organic Photoredox catalysis to produce hexane:

Hexane was prepare according to the followed procedure¹⁴: To a 50 mL round bottom flask was added 63 mg of diphenyl disulfide ((PhS)₂), 37 mg of N,N-diisopropylethylamine (i-Pr₂NET), 33 mg of 9-Mesityl-10-phenyl acridinium tetrafluoroborate (Mes-Acr-Ph), and 190 mg of heptanoic acid, followed by 3.9 mL 2,2,2-trifluoroethanol and 1 mL of ethyl acetate. The mixture was allowed to react at ambient temperature under irradiation for 48 h. The reaction mixture was passed through a plug of silica into a vial containing internal standard (methyl nonadecanoate) before GC-MS analysis (49 mg, 40% yield).

Scheme 6. Hydrodecarboxylation of Heptanoic Acid

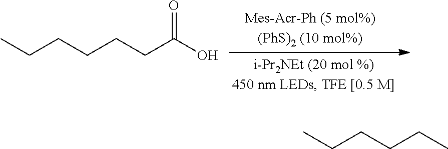

Synthesis of Methyl Heptanoate Via Esterification

Adding 20 g (0.15 mol) of heptanoic acid followed by 130 mL Methanolic HCl 1M to a 250 mL round bottom flask. The reaction was refluxed for 2 h at 85° C. Once the reaction complete and it was cooled to room temperature. The product was extracted with hexane then washed with 5% aqueous sodium carbonate and saturated aqueous sodium chloride. After drying over sodium sulfate, hexane was removed by rotary evaporator to obtain methyl heptanoate as colorless oil with grape smell (19.8 g, 90% yield). Methyl heptanoate: ¹H NMR (500 MHz, Chloroform-d) 6=3.64 (s, 3H), 2.31-2.25 (m, 2H), 1.59 (t, J=7.3, 2H), 1.34-1.21 (m, 6H), 0.90-0.81 (m, 3H).

Scheme 7. Synthesis of Methyl Heptanoate Via Esterification

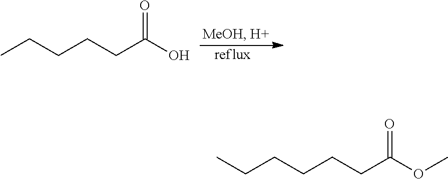

### REFERENCES FOR EXAMPLE 6

(1) D. J. Barrera and S. P. Mayfield, Handbook of Microalgal Culture: Applied Phycology and Biotechnology 27:532-544 (2013). (2) N. G. Schoepp, R. L. Stewart, V. Sun, A. J. Quigley, D. Mendola, S. P. Mayfield and M. D. Burkart, Bioresour Technol 166:273-281 (2014). (3) J. L. Blatti and M. D. Burkart, J Chem Educ 89:239-242 (2012). (4) N. G. Schoepp, W. Wong, S. P. Mayfield and M. D. Burkart, RSC Adv 5:57038-57044 (2015). (5) H. Nam, J. Choi and S. C. Capareda, Algal Res 17:87-96 (2016). (6) D. G. Hayes, Y. C. Bengtsson, J. M. V. Alstine and F. Setterwall, J Am Oil Chem Soc 75:1403-1409 (1998). (7) Bin Jiang, Yan Liu, L. Zhang, Yongli Sun, Y. Liu and X. Liu, J Chem Soc Pak 36:1013-1020 (2014). (8) R. G. Ackman, M. E. Retson, L. R. Gallay and F. A. Vandenheuvel, Can J Chem 39:1956-1963 (1961). (9) V. Benessere, M. E. Cucciolito, A. D. Santis, M. D. Serio, R. Esposito, F. Ruffo and R. Turco, J Am Oil Chem Soc 92:1701-1707 (2015). (10) A. Kockritz and A. Martin, Eur J Lipid Sci Technol 113:83-91 (2011). (11) A. A. H. Kadhum, B. A. Wasmi, A. B. Mohamad, A. A. Al-Amiery and M. S. Takriff, Res Chem Intermed 38:659-668 (2012). (12) B. M. Cochran, Synlett 27:245-248 (2016). (13) N. M. Noor, A. Sendijarevic, V. Sendijarevic, I. Sendijarevic, T. N. M. T. Ismail, M. A. M. Noor, Y. S. Klan and H. A. Hassan, J Am Oil Chem Soc 93:1529-1540 (2016). (14) J. D. Griffin, M. A. Zeller and D. A. Nicewicz, J Am Chem Soc 137:11340-11348 (2015).

What is claimed is:

1. A method of producing an algae free fatty acid composition comprising:
(a) contacting an algae fatty acid hydrolysate with a base thereby forming a soap composition;
(b) washing the soap composition with an organic solvent thereby removing one or more algae pigments to form a washed soap composition;
(c) contacting the washed soap composition with an acid thereby forming an algae free fatty acid composition.

2. The method of claim 1, wherein the algae free fatty acid composition comprises C16 free fatty acids.

3. The method of claim 2, wherein the C16 free fatty acids comprise palmitic (C16-0) free fatty acids and palmitoleic (C16-1) free fatty acids.

4. The method of claim 1, wherein the base is sodium hydroxide, potassium hydroxide, lithium hydroxide, or calcium hydroxide.

5. The method of claim 1, wherein the organic solvent is acetone, ether, or methyl tert-butyl ether.

6. The method of claim 1, wherein the one or more algae pigments are selected from the group consisting of a chlorophyll, a carotenoid, and a chlorophyll degradation product.

7. The method of claim 1, wherein the one or more algae pigments is selected from the group consisting of chlorophyll, astaxanthin, zeaxanthin, and canthaxanthin.

8. The method of claim 1, further comprising removing one or more saturated fatty acids from said algae fatty acid composition thereby forming an algae unsaturated fatty acid composition.

9. The method of claim 8, wherein the removing comprises contacting said algae fatty acid composition with methanol and applying temperature of about −15° C., thereby forming a crystalline fatty acid composition.

10. The method of claim 8, wherein the removing comprises contacting said algae fatty acid composition with urea at a temperature of about 4° C., thereby forming a crystalline fatty acid composition.

11. The method of claim 1, wherein the algae fatty acid hydrolysate is a microalgae fatty acid hydrolysate.

12. The method of claim 1, wherein the algae fatty acid hydrolysate is a Nannochloropsis sp. fatty acid hydrolysate, a Chlamydomonas sp. fatty acid hydrolysate, a Dunaliella sp. fatty acid hydrolysate, a Haematococcus sp. fatty acid hydrolysate, a Scenedesmus sp. fatty acid hydrolysate, a Diaphoreolis sp. fatty acid hydrolysate, or a Dunaliella sp. fatty acid hydrolysate.

13. The method of claim 1, wherein the algae fatty acid hydrolysate is a Nannochloropsis sp. fatty acid hydrolysate.

14. The method of claim 1, wherein the algae fatty acid hydrolysate is a C. reinhardtii fatty acid hydrolysate, D. satina fatty acid hydrolysate, H. pluvatis fatty acid hydrolysate, S. dimorphus fatty acid hydrolysate, D. viridis fatty acid hydrolysate, D. tertiolecta fatty acid hydrolysate, N. oculata fatty acid hydrolysate, or N. salina fatty acid hydrolysate.

15. The method of claim 1, wherein the algae fatty acid hydrolysate is a Cyanophyta fatty acid hydrolysate, a Prochlorophyta fatty acid hydrolysate, a Rhodophyta fatty acid hydrolysate, a Chlorophyta fatty acid hydrolysate, a Heterokontophyta fatty acid hydrolysate, a Tribophyta fatty acid hydrolysate, a Glaucophyta fatty acid hydrolysate, a Chlorarachniophyte fatty acid hydrolysate, a Euglenophyta fatty acid hydrolysate, a Euglenoid fatty acid hydrolysate, a Haptophyta fatty acid hydrolysate, a Chrysophyta fatty acid hydrolysate, a Cryptophyta fatty acid hydrolysate, a Cryptomonad fatty acid hydrolysate, a Dinophyta fatty acid hydrolysate, a Dinoflagellata fatty acid hydrolysate, a Prymnesiophyta fatty acid hydrolysate, a Bacillariophyta fatty acid hydrolysate, a Xanthophyta fatty acid hydrolysate, a Eustigmatophyta fatty acid hydrolysate, a Raphidophyta fatty acid hydrolysate, a Phaeophyta fatty acid hydrolysate, or a Phytoplankton fatty acid hydrolysate.

16. The method of claim 8, wherein the algae unsaturated fatty acid composition comprises palmitoleic acid (C16-1).

17. The method of claim 16, wherein the purity of the palmitoleic acid (C16-1) is from 85% to 90%.

18. The method of claim 8, further comprising contacting the algae unsaturated fatty acid composition with ozone followed by oxidation thereby forming an algae saturated dicarboxylic acid composition.

19. The method of claim 18, wherein the algae saturated dicarboxylic acid composition is an azelaic acid composition.

20. The method of claim 18, wherein the algae unsaturated fatty acid composition is contacted with ozone in an aqueous-organic solvent system.

21. The method of claim 20, wherein the aqueous-organic solvent system comprises aqueous acetonitrile.

22. The method of claim 18, wherein the oxidation is performed by the addition of aqueous sodium chlorite, followed by a quenching step with an aqueous reductant.

23. The method of claim 18, wherein the algae saturated dicarboxylic acid composition comprises heptanoic acid.

24. The method of claim 23, further comprising decarboxylating the heptanoic acid.

25. The method of claim 24, wherein the decarboxylating comprises catalytic hydrogenation or a light-dependent catalytic decarboxylation.

26. The method of claim 23, further comprising esterifying the heptanoic acid to heptanoyl methyl ester.

27. The method of claim 18, further comprising contacting the algae saturated dicarboxylic acid composition with a diol thereby forming an algae polyester polymer composition.

28. The method of claim 27, wherein the algae polyester polymer composition is formed using an esterification catalyst selected from the group consisting of a metal chloride, a metal oxide, a metal carboxylate, a metal alkoxide, and an organic acid.

29. The method of claim 28, wherein the algae saturated dicarboxylic acid composition is an algae azelaic acid composition and the diol is ethylene glycol.

30. The method of claim 28, wherein the algae saturated dicarboxylic acid composition is an algae oxalic acid composition, algae malonic acid composition, algae succinic acid composition, algae glutaric acid composition, algae adipic acid composition, algae 2,5-furandicarboxyic acid composition, algae pimelic acid composition, algae 3,3-dimethyl-1,2-cyclopropanedicarboxylic acid composition, algae suberic acid composition, algae azelaic acid composition, or algae sebacic acid composition.

31. The method of claim 27, wherein the diol is selected from the group consisting of ethylene glycol, 1,2 propanediol, 1,3-propanediol, glycerol, 1,3-butanediol, 1,4-butanediol, 2-methyl-1,3-propanediol, 2,3-butanediol, trimethylolpropane, 1,5-pentanediol, 1,6-hexanediol, 3-methyl-1,5-pentanediol, 1,7-heptanediol, 1,8-octanediol, 1,9-nonanediol, and 1,10-decanediol.

32. The method of one of claims 27 to 31, further comprising contacting said algae polyester polymer composition with a diisocyanate to form an algae polyurethane composition.

33. The method of claim 32, wherein the diisocyanate is selected from the group consisting of toluene diisocyanate (TDI), methylene diphenyl diisocyanate (MDI), hexamethylene diisocyanate (HDI), pentamethylene diisocyanate (PDI), 2,5-furandiisocyanate (FDI), heptamethylene diisocyanate (HPDI), hydrogenated MDI (H12MDI), and isophorone diisocyanate (IPDI).

34. The method of claim 32, wherein said algae polyurethane composition is formed using a catalyst selected from the group consisting of triethylenediamine, bis-(2-dimethylaminoethyl)-ether, N-methylmorpholine, N-ethylmorpholine, N,N,N′-trimethylisopropyl propylenediamine, dimethylcyclohexylamine, 1-methyl-4-dimethylaminoethylpiperazine, methoxypropyldimethylamine, N,N,N′,N′-tetramethyl-1,3-butanediamine, and dimethylethanolamine.

35. The method of claim 32, wherein the algae saturated dicarboxylic acid composition is an algae azelaic acid composition and said diol is an ethylene glycol.

36. A composition comprising an aqueous phase and an organic phase, wherein the aqueous phase comprises algae fatty acid alkali salts and the organic phase comprises one or more algae pigments.

37. The composition of claim 36, wherein the algae fatty acid alkali salt is an eicosapentaenoic acid (C20-5) alkali salt, a palmitoleic acid (C16-1) alkali salt, and/or a palmitic acid (C16-0) alkali salt.

38. The composition of claim 36 or claim 37, wherein the one or more algae pigments is selected from the group consisting of chlorophyll, astaxanthin, zeaxanthin, and canthaxanthin.

39. A composition comprising a palmitoleic acid (C16-1) alkali salt and a palmitic acid (C16-0) alkali salt.

40. A composition comprising a crystal phase and a liquid phase, wherein the crystal phase comprises a urea crystal and palmitic acid (C16-0), and the liquid phase comprises palmitoleic acid (C16-1).

41. A composition comprising palmitoleic acid (C16-1) and azelaic acid.

42. The composition of claim 41, further comprising malonic acid, heptanoic acid, and/or 2-methylfumaric acid.

43. The composition of claim 41 or claim 42, further comprising ozone.

44. A composition comprising an algae polyester polymer and one or more of malonic acid, a 2-methylfumaric acid ester, and/or a terminal heptanoic acid ester.

45. A composition comprising an algae polyurethane and one or more of malonic acid, a 2-methylfumaric acid ester, or a terminal heptanoic acid ester.

46. A polyurethane composition having a ¹³C to ¹²C fractional content (δ¹³C) of about −23‰ to about −12‰.
